SEQUENCE LISTING (2 sequences)

SEQ ID NO: 1 - protein, 1354 aa, Human
MSTGDSFETRFEKMDNLLRDPKSEVNSDCLLDGLDALVYDLDFPALRKNKNIDNFLSRYKDTINKIRDLRMKAEDYEVVKVIGRGAFGEVQLVRHKSTRKVYAMKLLSKFEMIKRSDSAFFWEERDIMAFANSPWVVQLFYAFQDDRYLYMVMEYMPGGDLVNLMSNYDVPEKWARFYTAEVVLALDAIHSMGFIHRDVKPDNMLLDKSGHLKLADFGTCMKMNKEGMVRCDTAVGTPDYISPEVLKSQGGDGYYGRECDWWSVGVFLYEMLVGDTPFYADSLVGTYSKIMNHKNSLTFPDDNDISKEAKNLICAFLTDREVRLGRNGVEEIKRHLFFKNDQWAWETLRDTVAPVVPDLSSDIDTSNFDDLEEDKGEEETFPIPKAFVGNQLPFVGFTYYSNRRYLSSANPNDNRTSSNADKSLQESLQKTIYKLEEQLHNEMQLKDEMEQKCRTSNIKLDKIMKELDEEGNQRRNLESTVSQIEKEKMLLQHRINEYQRKAEQENEKRRNVENEVSTLKDQLEDLKKVSQNSQLANEKLSQLQKQLEEANDLLRTESDTAVRLRKSHTEMSKSISQLESLNRELQERNRILENSKSQTDKDYYQLQAILEAERRDRGHDSEMIGDLQARITSLQEEVKHLKHNLEKVEGERKEAQDMLNHSEKEKNNLEIDLNYKLKSLQQRLEQEVNEHKVTKARLTDKHQSIEEAKSVAMCEMEKKLKEEREAREKAENRVVQIEKQCSMLDVDLKQSQQKLEHLTGNKERMEDEVKNLTLQLEQESNKRLLLQNELKTQAFEADNLKGLEKQMKQEINTLLEAKRLLEFELAQLTKQYRGNEGQMRELQDQLEAEQYFSTLYKTQVKELKEEIEEKNRENLKKIQELQNEKETLATQLDLAETKAESEQLARGLLEEQYFELTQESKKAASRNRQEITDKDHTVSRLEEANSMLTKDIEILRRENEELTEKMKKAEEEYKLEKEEEISNLKAAFEKNINTERTLKTQAVNKLAEIMNRKDFKIDRKKANTQDLRKKEKENRKLQLELNQEREKFNQMVVKHQKELNDMQAQLVEECAHRNELQMQLASKESDIEQLRAKLLDLSDSTSVASFPSADETDGNLPESRIEGWLSVPNRGNIKRYGWKKQYVVVSSKKILFYNDEQDKEQSNPSMVLDIDKLFHVRPVTQGDVYRAETEEIPKIFQILYANEGECRKDVEMEPVQQAEKTNFQNHKGHEFIPTLYHFPANCDACAKPLWHVFKPPPALECRRCHVKCHRDHLDKKEDLICPCKVSYDVTSARDMLLLACSQDEQKKWVTHLVKKIPKNPPSGFVRASPRTLSTRSTANQSFRKVVKNTSGKTS

SEQ ID NO: 2 - DNA, 4065 bp, Human
atgtcgactggggacagttttgagactcgatttgaaaaaatggacaacctgctgcgggatcccaaatcggaagtgaattcggattgtttgctggatggattggatgctttggtatatgatttggattttcctgccttaagaaaaaacaaaaatattgacaactttttaagcagatataaagacacaataaataaaatcagagatttacgaatgaaagctgaagattatgaagtagtgaaggtgattggtagaggtgcatttggagaagttcaattggtaaggcataaatccaccaggaaggtatatgctatgaagcttctcagcaaatttgaaatgataaagagatctgattctgcttttttctgggaagaaagggacatcatggcttttgccaacagtccttgggttgttcagcttttttatgcattccaagatgatcgttatctctacatggtgatggaatacatgcctggtggagatcttgtaaacttaatgagcaactatgatgtgcctgaaaaatgggcacgattctatactgcagaagtagttcttgcattggatgcaatccattccatgggttttattcacagagatgtgaagcctgataacatgctgctggataaatctggacatttgaagttagcagattttggtacttgtatgaagatgaataaggaaggcatggtacgatgtgatacagcggttggaacacctgattatatttcccctgaagtattaaaatcccaaggtggtgatggttattatggaagagaatgtgactggtggtcggttggggtatttttatacgaaatgcttgtaggtgatacacctttttatgcagattctttggttggaacttacagtaaaattatgaaccataaaaattcacttacctttcctgatgataatgacatatcaaaagaagcaaaaaaccttatttgtgccttccttactgacagggaagtgaggttagggcgaaatggtgtagaagaaatcaaacgacatctcttcttcaaaaatgaccagtgggcttgggaaacgctccgagacactgtagcaccagttgtacccgatttaagtagtgacattgatactagtaattttgatgacttggaagaagataaaggagaggaagaaacattccctattcctaaagctttcgttggcaatcaactaccttttgtaggatttacatattatagcaatcgtagatacttatcttcagcaaatcctaatgataacagaactagctccaatgcagataaaagcttgcaggaaagtttgcaaaaaacaatctataagctggaagaacagctgcataatgaaatgcagttaaaagatgaaatggagcagaagtgcagaacctcaaacataaaactagacaagataatgaaagaattggatgaagagggaaatcaaagaagaaatctagaatctacagtgtctcagattgagaaggagaaaatgttgctacagcatagaattaatgagtaccaaagaaaagctgaacaggaaaatgagaagagaagaaatgtagaaaatgaagtttctacattaaaggatcagttggaagacttaaagaaagtcagtcagaattcacagcttgctaatgagaagctgtcccagttacaaaagcagctagaagaagccaatgacttacttaggacagaatcggacacagctgtaagattgaggaagagtcacacagagatgagcaagtcaattagtcagttagagtccctgaacagagagttgcaagagagaaatcgaattttagagaattctaagtcacaaacagacaaagattattaccagctgcaagctatattagaagctgaacgaagagacagaggtcatgattctgagatgattggagaccttcaagctcgaattacatctttacaagaggaggtgaagcatctcaaacataatctcgaaaaagtggaaggagaaagaaaagaggctcaagacatgcttaatcactcagaaaaggaaaagaataatttagagatagatttaaactacaaacttaaatcattacaacaacggttagaacaagaggtaaatgaacacaaagtaaccaaagctcgtttaactgacaaacatcaatctattgaagaggcaaagtctgtggcaatgtgtgagatggaaaaaaagctgaaagaagaaagagaagctcgagagaaggctgaaaatcgggttgttcagattgagaaacagtgttccatgctagacgttgatctgaagcaatctcagcagaaactagaacatttgactggaaataaagaaaggatggaggatgaagttaagaatctaaccctgcaactggagcaggaatcaaataagcggctgttgttacaaaatgaattgaagactcaagcatttgaggcagacaatttaaaaggtttagaaaagcagatgaaacaggaaataaatactttattggaagcaaagagattattagaatttgagttagctcagcttacgaaacagtatagaggaaatgaaggacagatgcgggagctacaagatcagcttgaagctgagcaatatttctcgacactttataaaacccaggtaaaggaacttaaagaagaaattgaagaaaaaaacagagaaaatttaaagaaaatacaggaactacaaaatgaaaaagaaactcttgctactcagttggatctagcagaaacaaaagctgagtctgagcagttggcgcgaggccttctggaagaacagtattttgaattgacgcaagaaagcaagaaagctgcttcaagaaatagacaagagattacagataaagatcacactgttagtcggcttgaagaagcaaacagcatgctaaccaaagatattgaaatattaagaagagagaatgaagagctaacagagaaaatgaagaaggcagaggaagaatataaactggagaaggaggaggagatcagtaatcttaaggctgcctttgaaaagaatatcaacactgaacgaacccttaaaacacaggctgttaacaaattggcagaaataatgaatcgaaaagattttaaaattgatagaaagaaagctaatacacaagatttgagaaagaaagaaaaggaaaatcgaaagctgcaactggaactcaaccaagaaagagagaaattcaaccagatggtagtgaaacatcagaaggaactgaatgacatgcaagcgcaattggtagaagaatgtgcacataggaatgagcttcagatgcagttggccagcaaagagagtgatattgagcaattgcgtgctaaacttttggacctctcggattctacaagtgttgctagttttcctagtgctgatgaaactgatggtaacctcccagagtcaagaattgaaggttggctttcagtaccaaatagaggaaatatcaaacgatatggctggaagaaacagtatgttgtggtaagcagcaaaaaaattttgttctataatgacgaacaagataaggagcaatccaatccatctatggtattggacatagataaactgtttcacgttagacctgtaacccaaggagatgtgtatagagctgaaactgaagaaattcctaaaatattccagatactatatgcaaatgaaggtgaatgtagaaaagatgtagagatggaaccagtacaacaagctgaaaaaactaatttccaaaatcacaaaggccatgagtttattcctacactctaccactttcctgccaattgtgatgcctgtgccaaacctctctggcatgtttttaagccaccccctgccctagagtgtcgaagatgccatgttaagtgccacagagatcacttagataagaaagaggacttaatttgtccatgtaaagtaagttatgatgtaacatcagcaagagatatgctgctgttagcatgttctcaggatgaacaaaaaaaatgggtaactcatttagtaaagaaaatccctaagaatccaccatctggttttgttcgtgcttcccctcgaacgctttctacaagatccactgcaaatcagtctttccggaaagtggtcaaaaatacatctggaaaaactagttaa

(19) United States
(12) Patent Application Publication
(10) Pub. No.: US 2006/0142193 A1
(43) Pub. Date: Jun. 29, 2006

(54) Selective inhibition of rock1 in cardiac therapy

(75) Inventors: Lei Wei, Fishers, IN (US); Robert J. Schwartz, Houston, TX (US); Jiang Chang, Houston, TX (US); Mark Entman, Houston, TX (US)

Correspondence Address: FULBRIGHT & JAWORSKI, LLP, 1301 MCKINNEY, SUITE 5100, HOUSTON, TX, 77010-3095, US

(73) Assignee: BAYLOR COLLEGE OF MEDICINE, Houston, TX (US)

(21) Appl. No.: 11/270,653

(22) Filed: Nov. 9, 2005

Related U.S. Application Data
(60) Provisional application No. 60/626,390, filed on Nov. 9, 2004.

(51) Int. Cl. A61K 38/17; A61K 48/00

(52) U.S. Cl. 514/12; 514/44

(57) ABSTRACT

The present invention is directed to the treatment and/or prevention of disease as it relates to Rho kinase. In specific embodiments, disease is treated and/or prevented through the administration of an agent that selectively inhibits ROCK1. In specific embodiments, it inhibits ROCK1 and not ROCK2. In other specific embodiments, the disease is cardiac disease.

The present invention claims priority to U.S. Provisional Patent Application Ser. No. 60/626,390, filed Nov. 9, 2004, which is incorporated by reference herein in its entirety.

The present invention used in part funds from NIH National Heart, Lung and Blood Institute Grant Nos. HL 64356-03, P01-HL49953, R01-HL72897, and P01-HL42550 The United States Government may have certain rights in the invention.

### FIELD OF THE INVENTION

The present invention relates to the fields of cell biology, molecular biology, and medicine. Specifically, the invention is directed to diagnosis and/or treatment of cardiac disease.

### BACKGROUND OF THE INVENTION

Heart failure is the leading cause of combined morbidity and mortality in the United States and other developed industrial nations. It remains an incurable disease process with an estimated two-year mortality of 30-50% for the patients with advanced disease. Although great advances in the treatment for failing heart have been made, our understanding of the molecular mechanism leading to heart failure is still limited. It is evident, however, that severe heart failure is associated with striking decreases in the expression of cardiac specific genes (Razeghi et al., 2002; Hwang et al., 2002; Barrans et al., 2002).

Heart failure is characterized by a relentless progression: a relatively long interval (several years) exists between the initial events causing myocardial damage and the final state termed dilated cardiomyopathy, in which heart chambers become markedly enlarged and contractile function deteriorates. The molecular and cellular mechanisms that mediate the pathogenesis of heart failure during this long interval are poorly understood.

A commonly accepted paradigm for the development of heart failure divides the pathological process into two distinct stages; an initial compensatory hypertrophy in response to excess hemodynamic loading, followed by a critical transition to decompensated failure under persistent stress (Dorn et al., 2003; Sawyer et al., 2002; Mann, 2003; Bueno et al., 2000; Arai et al., 1994; Sadoshima and Izumo, 1997; Sussman et al., 2002; Adams et al., 1998; Hoshijima and Chien, 2002; Chien, 1999; Wang, 2001; Sabri et al., 2003). The most characteristic events occurring during pathological remodeling include, for example, a change in gene expression profiles from adult to a “fetal-like” programs, increase in myocyte size and protein content, induction of sarcomeric disorganization, induction of interstitial fibrosis, depressed myocyte contractility, loss of intercellular conduction, and myocyte cell loss. Recent progress in molecular genetics and cellular/organ physiology has provided powerful tools to dissect molecular components involved in each aspect of the remodeling processes and to establish the cause/effect relationship between different signaling pathways and specific pathological processes in the heart.

Roles of anoptosis in heart failure. In many transgenic animal models of dilated cardiomyopathy, ventricular dysfunction has been attributed to depressed myocyte contractility. However, recent studies indicate that apoptotic myocyte death is also a determinant factor involved in the transition to failure (Kang and Izumo, 2000; Yussman et al., 2002; Nadal-Ginard et al., 2003; Wencker et al., 2003; Yamamoto et al., 2003; Narula et al., 2001; Olivetti et al., 1997). Apoptosis has been demonstrated in human heart failure, with the reported prevalence varying widely, but more recent work has supported a prevalence of less than 1%, consistent with slow progression of heart failure (Narula et al., 1999; Narula et al., 1996; Ba\lankenberg et al., 1999; Elsasser et al., 2000).

FIG. 1 provides a schematic presentation of proteolytic activation of the caspase cascade in heart failure. Apoptosis is a highly orchestrated form of programmed cell death, and results from the activation of caspases, which are specialized aspartate-directed proteases. Two major pathways lead to the activation of the caspase cascade. Both pathways lead to the activation of caspase 3, a key executing caspase, which cleaves various subcellular cytoplasmic proteins and fragments nuclear DNA.

An increasing number of apoptotic inducers (TNFα, Gαq, plasma Fas ligand, etc.), survival factors (IGF-1, Akt, interleukin-6 and its receptor gp130, etc.), and regulatory factors (BclXL, Bcl-2, Bax, etc.) have been reported to influence myocyte apoptosis in heart failure through modulating the activity of the caspase cascade. However, our knowledge of the mechanism and regulation of apoptosis in myocyte is still limited. Thus, understanding the basic processes involved in progression of apoptosis may offer new possibilities to treat heart failure. It has been shown that in favorable conditions, such as with left ventricular assist devices (LVAD) support, the apoptotic process in failing cardiomyocytes is markedly attenuated (Narula et al., 2001; Elsasser et al., 2000), indicating the potential feasibility of reversal of heart failure.

Roles of Caspase 3 in heart failure. Caspase 3 is a key executing caspase for carrying out apoptosis in eukaryotic cells (Thornberry and Lazebnik, 1998). Caspase 3 expression is increased in association with heart failure and apoptosis in experimental animals (Sabbah, 2000). It is also found in its activated form in the myocardium of end-stage heart failure patients (narula et al., 1996; Blankenberg et al., 1999). Cardiac specific overexpression of caspase 3 in transgenic mice induces transient depression of cardiac function and abnormal nuclear-and myofibrillar ultrastructural damage, but does not trigger a full apoptotic response in the cardiomyocyte (Condorelli et al., 2001). However, overexpression of caspase 3 leads to a significant increase in infarct size after ischemic-reperfusion (Condorelli et al, 2001). Although these studies strongly suggest a role for caspase 3 in heart failure, the extent of its contribution to the initiation and progression of heart failure as well as the mechanisms involved in myocardial structure and function changes induced by caspase 3 still remain poorly understood.

Identification of endogenous substrates for caspase 3 has provided important clues to its molecular role in apoptosis. The optimal recognition motif for caspase 3 is DEVD (Thornberry et al., 2000; Thornberry et al., 1997), which is similar or identical to the cleavage sites in several known in vitro or vivo substrates of caspase 3. Caspase 3 has been shown to cleave several cardiac contractile proteins, including ventricular essential myosin light chain (Moretti et al., 2002), cardiac α-actin, α-actinin, and cardiac troponin T (Communal et al., 2002), providing a potential mechanism through which activation of caspase 3 contributes to contractile dysfunction before cell death. Moreover, several protein kinases including PKCδ (Kaul et al., 2003; Anantharam et al., 2002) and Mst1 (Lee et al., 2001) have been identified as caspase 3 substrates in cardiomyocytes. Both kinases have been shown to be important mediators of apoptosis in cardiomyocytes (Yamamoto et al., 2003; Schaffer et al., 2003).

Role of RhoA in cardiac hvpertrophy and heart failure. Rho GTPase family proteins, which include RhoA, Rac1 and Cdc42, control a wide variety of cellular processes such as cell morphology, motility, proliferation, differentiation and apoptosis (Hall, 1994; Van Aelst and D'Souza-Schorey, 1997). Recent studies suggest that RhoA is also involved in cardiac hypertrophy. In cultured cardiomyocytes, RhoA is required for hypertrophic signals induced by α1-adrenergic agonist phenylephrine (Hoshijima et al., 1998), angiotensin II (Aoki et al., 1998) and mechanical stress (Aikawa et al., 1999). RhoA expression is up-regulated in the failing heart of Dahl salt-sensitive hypertensive rats (Kobayashi et al., 2002). Cardiac-specific overexpression of RhoA in mice leads to sinus and atrioventricular (AV) nodal dysfunction and heart failure (Sah et al., 1999). Statins, inhibitors of 3-hydroxy-3-methylglutaryl-CoA reductase, have been shown to prevent the development of cardiac hypertrophy in vivo and in vitro. This possibly occurs in part through inhibition of membrane translocation of Rho proteins, as statins block Rho isoprenylation (Takemoto et al., 2001; Patel et al., 2001; Laufs et al., 2002).

The signaling pathways activated by RhoA to promote cardiomyocyte hypertrophy in vitro and in vivo are not well understood. It was determined that an organized cytokeletal structure is required for activation of SRF-dependent gene expression by RhoA in cultured neonatal cardiomyocytes (Wei et al., 2001). Other studies in cultured cardiomyocytes have suggested that PKN mediates RhoA-dependent activation of SRF (Morissette et al., 2000) and that RhoA promotes GATA-4-dependent gene regulation via a p38 mitogen-activated protein kinases (MAPK)-dependent pathway (Yanazume et al., 2002; Charron et al., 2001). Another potential mediator of RhoA in promoting cardiomyocyte hypertrophy is Rho kinase as described below.

WO 03/080610 relates to imidazopyridine derivatives as kinase inhibitors, such as ROCK inhibitors, and methods for inhibiting the effects of ROCK1 and/or ROCK2.

### BRIEF SUMMARY OF THE INVENTION

The present invention concerns the treatment of cardiac failure. It is known that a pathological cardiac hypertrophy due to pressure overload is initially a compensatory response, but eventually leads to decompensation, resulting in heart failure or sudden death. In a specific embodiment of the invention, apoptosis plays a role in cardiac failure. In a specific embodiment, ROCK1 plays an important role in the transition from compensated cardiac hypertrophy to heart failure, and ROCK1 is a critical regulatory factor of cardiomyocyte apoptosis.

The present inventors demonstrate that patients with end-stage heart failure demonstrated marked ROCK-1 cleavage that was reversed in hearts with left ventricular assist device (LVAD). ROCK-1 cleavage was detected in cultured cardiomyocytes subjected to apoptotic stimuli. ROCK-1 fragmentation was also observed in the bi-transgenic Gq-HGK mice, which displayed the most severe cardiomyopathy. An activated ROCK-1 mutant strongly promoted caspase 3 activation by inhibiting the cell survival factor AKT through increased PTEN activity. Blocked ROCK-1 expression by siRNA attenuated caspase activation. Lines of ROCK-1 null mice displayed a marked reduction in apoptosis associated with pressure overload. ROCK-1 cleavage amplifies apoptotic signals and strongly promotes end stage heart failure, in particular aspects related to the invention.

In specific embodiments of the invention, modified ROCK-1, such as truncated ROCK-1, for example, which is a catalytically active enzyme, (Coleman et al., 2001; Sebbagh et al., 2001), was sufficient to activate a caspase cascade and lead to a potential positive feed-forward loop, promoting apoptosis. The present inventors further identified the activation of PTEN (phosphatase and tensin homolog deleted on chromosome ten) and subsequent inhibition of the AKT pathway as a critical pro-apoptotic mechanism. These studies provide novel evidence that caspase 3-mediated ROCK-1 cleavage activates an important apoptotic pathway in heart failure.

In particular, as shown herein, ROCK1 (but not ROCK2) is a substrate of caspase 3 in human failing hearts and in cultured apoptotic cardiomyocytes. Expression of a ROCK1 mutant (ROCK1Δ1), which closely mimics the caspase 3 cleaved form, leads to activation of caspase 3 in cultured cardiomyocytes. In addition, serum response factor (SRF), which plays an important role in the regulation of cardiac gene expression in mammalian heart, is also a substrate of caspase 3 in human failing hearts. Moreover, phosphorylation of SRF by ROCK1Δ1 facilitates SRF cleavage by caspase 3 in vitro. In specific embodiments of the invention, these observations indicate that there is a novel mechanism contributing to the slow progression of heart failure: activated caspase 3 cleaves ROCK1 and generates an active form of this kinase, thereby leading to myocyte apoptosis and the phosphorylation and alteration in the activity and/or expression of many cardiac proteins, including SRF, for example.

Consistent with the observations in human failing hearts and in cultured cardiomyocytes, ROCK1 homozygous-deficient mice develop cardiac hypertrophy in response to pressure overload, but exhibit significantly reduced hypertrophic marker induction, reduced myocyte apoptosis, reduced interstitial fibrosis, and improved cardiac contractile functions, compared to control mice.

In a particular embodiment of the invention, it is demonstrated how ROCK1 activation by caspase 3 cleavage leads to cardiomyocyte apoptosis in cultured cardiomyocytes. In a specific embodiment, caspase 3 cleavage resistant mutant (ROCK1_{D1113A}) or a kinase defective mutant (ROCK1_{KD}) protects cardiomyocytes from apoptosis. In another specific embodiment ROCK1Δ1 induces cardiomyocyte apoptosis through activation of the caspase cascade. In an additional specific embodiment, ROCK1Δ1 facilitates cleavage of SRF by caspase 3. In an additional specific embodiment, ROCK1Δ1 induces myocyte apoptosis through repressing activity of critical survival signaling pathways.
In another particular embodiment, it is demonstrated how ROCK1 activation by caspase 3 cleavage leads to the progression of heart failure. This may be demonstrated through an inducible bi-transgenic gain-of-function approach, for example. In a specific embodiment, cardiac-specific inducible expression of ROCK1Δ1 induces cardiomyocyte apoptosis and heart failure in intact animals.

In an additional particular embodiment, the role of ROCK1 is demonstrated in mediating heart failure under cardiac conditions associated with caspase 3 activation using ROCK1-deficient mice, cardiac-specific ROCK1-deficient mice, and mice with a knockin mutation in the ROCK1 gene resistant to caspase 3 cleavage. In a specific embodiment, ROCK1 deficiency inhibits cardiomyocyte apoptosis and heart failure under the pathological conditions in which apoptosis plays a significant role in the development of heart failure. In an additional specific embodiment, the in vivo knockin mutation of the endogenous ROCK1, resistant to caspase 3 cleavage, inhibits cardiomyocyte apoptosis and heart failure under these conditions.

In a particular embodiment, the present invention is directed to a system, method, and/or compositions related to diagnosis of cardiac failure and/or cardiac disease associated with, or comprising, elevated levels of cleaved Rho kinase, particularly by caspases during apoptosis. In specific embodiments, the cleavage of Rho kinase is diagnosed, prevented, delayed, ameliorated (although not necessarily completely), inhibited (although not necessarily completely), or a combination thereof. In specific embodiments, the cleavage of Rho kinase is inhibited, prevented, delayed, or ameliorated at least partially.

In an embodiment of the present invention, there is a method of preventing or delaying apoptosis of a cell, comprising the step of delivering an agent to the cell, wherein the agent selectively inhibits ROCK1. In specific embodiments, the method is further defined as the agent selectively inhibiting ROCK1 over ROCK2. In particular embodiments, the agent comprises a nucleic acid, a peptide, a polypeptide, or a mixture thereof. In a specific embodiment, the agent comprises part or all of the pleckstrin homology (PH) domain of ROCK1, which in specific embodiments may further be defined as residues 1118 to 1317 of SEQ ID NO:1. In further specific embodiments, the nucleic acid comprises antisense RNA or siRNA. In additional specific embodiments, the agent comprises a peptide from part or all of the PH domain of ROCK1. The cell may be a heart cell, lung cell, liver cell, kidney cell, mesenchymal stem cell, fibroblast cell, myofibroblast cell, or stem cell.

As used herein, the term “selectively inhibiting” refers to the preferential inhibition of ROCK 1 instead of other molecules, such as other Rho kinase-related molecules, including ROCK2. The selective inhibition may be complete, such as the agent being ineffective against ROCK2, including having no detectable effect on ROCK2. In alternative embodiments, the selective inhibition permits a minor amount of inhibition of ROCK2, although significantly reduced compared to the inhibition of ROCK1. In specific embodiments, ROCK1 inhibition is about 5-fold greater than ROCK 2, is about 10-fold greater than ROCK2, is about 50-fold greater than ROCK2, is about 100-fold greater than ROCK2, is about 500-fold greater than ROCK2, is about 1000-fold greater than ROCK2, is about 10,000-fold greater than ROCK2, and so forth.

In one embodiment of the present invention, there is a method of preventing or delaying apoptosis of a cell, comprising the step of delivering an agent to the cell, wherein said agent inhibits ROCK1 but does not inhibit ROCK2. In a specific embodiment, inhibiting of ROCK1 is further defined as inhibiting activity of ROCK1 in said cell; inhibiting expression of ROCK1 in said cell; inhibiting cleavage of ROCK1 in said cell; or a combination thereof. In a specific embodiment, cleavage of ROCK1 is by a caspase, such as, for example, caspase 3. In specific embodiments, the cell is from cardiac tissue. The cell may be a cardiomyocyte. The cell may be in a mammal, such as, for example, a human.

In particular embodiments, methods of the invention are further defined as inhibiting fibrosis in cardiac tissue of the human. The inhibitor(s) may be selected from the group consisting of a small molecule, a nucleic acid, a polypeptide, or a mixture thereof. In specific embodiments, the nucleic acid molecule is DNA, such as antisense ROCK1 DNA. In other specific embodiments, the nucleic acid molecule is RNA, such as siRNA. The antisense ROCK1 DNA molecule may be directed to any region so long as it decreases expression of ROCK1 by greater than 1-fold.

In other embodiments, the methods and compositions of the present invention are utilized for a human that has cardiac disease. In specific embodiments, the human is susceptible to cardiac disease. In specific embodiments, the methods of the invention further comprise the step of administering an additional cardiac disease therapy to the human, such as drug therapy, device therapy, gene therapy, nutritional and exercise therapy, or a combination thereof.

In particular embodiments, the ROCK1-inhibiting step permits maintaining the adaptive response of cardiomyocyte enlargement. The inhibiting step may be further defined as not adversely affecting the ability of the individual to develop enlarged cardiomyocytes in response to pressure overload.

In an additional embodiment, there is a method of treating cardiac failure in an individual, said heart failure the direct or indirect result of cleavage of Rho kinase in at least one cardiac cell of the individual, comprising administering to the individual a therapeutically effective amount of an inhibitor that inhibits ROCK1 without inhibiting ROCK2. The method may further comprise the step of providing an additional therapy, such as drug therapy, device therapy, gene therapy, nutritional and exercise therapy, or a combination thereof. In a specific embodiment, the device therapy comprises administration of a left ventricular assist device to the individual.

In an additional embodiment of the present invention, there is a kit for the treatment of cardiac failure in an individual, comprising an agent that inhibits ROCK1 but not ROCK2.

In another embodiment of the present invention, there is a transgenic mouse, comprising at least one defective allele of ROCK1. The transgenic mouse may be further defined as having two defective alleles of ROCK1.

In an additional embodiment, there is a method of identifying a compound for treatment and/or prevention of cardiac disease, comprising the steps of providing a cardiac cell; and administering to the cell a test compound, wherein when the test compound inhibits expression, activity, and/or cleavage of ROCK1 but not ROCK2 in said cell, said test compound is said compound for treatment and/or prevention of cardiac disease. In a specific embodiment, there is a therapeutically effective amount of said identified compound is administered to an individual having cardiac disease or being susceptible to cardiac disease. In another specific embodiment, the cell is in an animal.

In a particular embodiment, there is a compound for treatment and/or prevention of cardiac disease, obtained by exemplary methods as described herein.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1 illustrates an exemplary schematic presentation of proteolytic activation of the caspase cascade in heart failure.

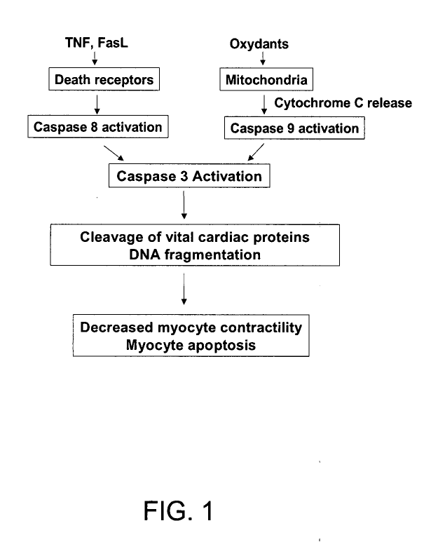

FIG. 2 provides an exemplary model for the present invention. Pressure overload activates RhoA signaling pathway, which in turn transiently activates ROCK1 as well as ROCK2.

FIG. 3 shows expression analysis of ROCK1 and ROCK2 in ROCK1 homozygous knockout adult hearts. Two wild-type littermates (ROCK1+/+) were used as controls. Equal amounts of proteins from heart homogenates were analyzed by Western blotting using anti-ROCK1 or anti-ROCK2 directed against the coiled-coil region of ROCK1 or ROCK2 respectively.

FIGS. 4A-4C show that cardiac hypertrophy develops in response to pressure overload in ROCK1^{-/-} mice. FIG. 4A shows heart sections from ROCK1^{-/-} and control mice after three-week aortic banding. Bar, 1 mm. FIG. 4B shows quantitation of heart/body weight ratios from ROCK1^{-/-} and control mice after three-week aortic banding (n=5 for each group). *P<0.05 vs. sham. FIG. 4C shows cardiomyocyte diameters from ROCK1^{-/-} and control mice after three-week aortic banding. Myocyte diameter was measured using transnuclear width at the mid-ventricular level (n=200 for each condition). *P<0.05 vs. sham.

FIG. 5 shows real-time RT-PCR analysis of cardiac hypertrophic markers. RNA samples were prepared from ROCK1^{-/-} and control hearts after three-week aortic banding (n=3-4 for each group). Quantitative RT-PCR analysis was performed using the ABI Prism 7700 sequence detection system (Perkin Elmer). The levels of the transcripts were normalized to that of GAPDH. *P<0.05 vs. control.

FIG. 6 shows that pressure overload causes less cell death and interstitial fibrosis in the myocardium of ROCK1^{-/-} mice. FIG. 6A shows TUNEL-positive myocytes in the left ventricular myocardium of ROCK1^{-/-} (n=5) and control mice (n=4) three weeks after aortic banding. The number of TUNEL-positive myocyte nuclei and the total number of myocyte nuclei (DAPI-staining) were manually counted (n=10,000 for each condition). Only nuclei that were clearly located in areas with a true cross section of myocytes (anti-αMHC staining) were scored. *P<0.05 vs. control. FIG. 6B shows picric acid Sirius red staining of heart sections three weeks after aortic banding. Arrow indicates fibrosis. Bar, 20 μm.

FIGS. 7A and 7B show that ROCK1 is cleaved in human failing hearts. FIG. 7A is a schematic diagram of ROCK1 cleavage. The consensus recognition sequence for caspase 3 in human ROCK1 (DETD1113) is conserved in mouse and rat, and is not present in ROCK2. FIG. 7B provides representative Western blots of hearts samples. Cleavage of ROCK1, caspase 3, and poly(ADP-ribose) polymerase (PARP) (a well established caspase 3 substrate) was observed in human failing hearts, but not in normal hearts or in failing hearts unloaded by LVAD support. This suggests that caspase 3 activation relates to myocardial mechanical overload.

FIGS. 8A-8E show that ROCK-1 was cleaved by caspase 3 in apoptotic cardiomyocytes and in transgenic heart failure animal models. In FIG. 8A Western blot of whole cell lysates from untreated and doxorubicin-treated (Dox) neonatal rat cardiomyocytes revealed cleavages of ROCK-1 and PARP. Caspase 3 inhibitor, Z-VAD, blocked the cleavage. In FIG. 8B, there is a schematic diagram of conditional activation of caspase 3 with addition of CID; Chemical inducer binding domain (CBD). In FIG. 8C, there is a Western blot of whole cell lysates from neonatal rat cardiomyocytes infected with the adenovirus, Ad-iCaspase 3 encoding a conditional caspase 3, that revealed the cleavage of ROCK-1 only in the presence of CID, which activates the conditional caspase 3. In FIGS. 8D and 8E, myocardial tissues from three transgenic mouse lines were evaluated by Western blot for caspase 3 activity and ROCK-1. A significant increase in caspase 3 activity was observed in hearts from bi-transgenic mice paralleled with a 130 kDa fragment of ROCK-1. No obvious cleavage was found in HGK or Gq mice, although there was a slight increase in caspase 3 activity revealed in Gq mice.

FIGS. 9A-9C show that ROCK-1 active mutant, ROCKΔ1, was sufficient to induce caspase 3 activation and myocytes apoptosis in neonatal rat cardiomyocytes. In FIG. 9A, there is a schematic diagram of caspase 3 sensor: a caspase 3 specific cleavage site is located between EYFP and NES. When caspase 3 is inactive, the dominant NES targets EYFP to the cytosol. Upon induction of apoptosis, the export signal is removed by active caspase 3, which triggers the redistribution of EYFP from cytosol to the nucleus via NLS. In FIG. 9B, there is a representative image showing myocytes subjected to ROCKD1 developed apoptosis characterized by accumulated EYFP in myocyte nuclei and disorganized myofilaments. (if shown in color photos: green represents EYFP-fusion protein; red represents rhodamine-conjugated phalloidin staining for F-actin; and blue represents DAPI nucleus staining. In FIG. 9C, the level of apoptosis was evaluated by the percentage of transfected cardiomyocytes exhibiting caspase 3 activation (nuclear localization of EYFP-fusion protein). Results are the average ±standard error of four separate experiments. *P<0.001 vs. control group.

FIGS. 10A and 10B show that SRF cleavage by caspase 3 in failing human hearts can be reversed by LVAD support. FIG. 10A illustrates a schematic diagram of SRF cleavage. The consensus recognition sequences for caspase 3 in human SRF (EETD245 and SESD254) are conserved in mouse and rat. FIG. 10B shows representative Western blots of hearts samples. Full length SRF was markedly reduced in failing human hearts. These alterations were attenuated with LVAD support. Site-directed mutagenesis revealed that the cleavage by caspase 3 occurs at D245 and D254, generating two different 32-kDa fragments, and that the 55-kDa fragment may be generated by other proteases.

FIG. 11 shows that phosphorylation of SRF by active ROCK1 facilitates SRF cleavage by caspase 3 in vitro. Equal amounts of SRF were incubated with active caspase 3 in the presence or absence of ROCK1Δ1. The full-length level was markedly decreased in the presence of active ROCK1, while the level of the cleaved fragment recognized by the anti-SRF-C antibody was not increased, most likely due to further degradation by caspase 3. In addition, phosphorylation of SRF by ROCK1 did not affect the cleavage sites.

FIGS. 12A-12E shows cardiac-specific and ligand-inducible expression of human growth hormone (hGH) in the bi-transgenic mouse hearts. In FIG. 12A, there is a schematic diagram of the cardiac-specific bi-transgenic system. Transgenic mice with the Glp65 regulator placed under the transcriptional control of the αMHC promoter were generated. The target transgenic line, 17×4-TATA-hGH, was previously generated (Wang et al., 1997). In FIG. 12B, in situ hybridization analysis of inducible expression of hGH is provided. Expression of the transgene is induced in all 4 chambers of the bi-transgenic hearts by administration of RU486 for 4 days. Bar, 1 mm. FIG. 12C shows cardiac-specific inducible expression of hGH in bitransgenic mouse hearts. The mRNA transcript of hGH was detected by RT-PCR only in the bi-transgenic hearts after RU486 administration for 4 days. FIG. 12D shows switching on or off hGH expression by administration (4 days) or withdrawal (7 days) (*) of RU486. The serum level of hGH was measured by radioimmunoassay. FIG. 12E demonstrates dose-dependent induction of hGH by RU486 for 4 days.

FIG. 13 provides an exemplary diagram of the design of study of one embodiment of the invention.

FIG. 14 shows one embodiment of potential signaling pathways mediating ROCK1-induced myocyte apoptosis.

FIGS. 15A-15C show that TAT-SRF is able to enter into cultured cells in a concentration-dependent fashion and is preferentially localized in the nucleus of cardiomyocytes. FIG. 15A shows that TAT-SRF was labeled with FITC and added into the culture medium of neonatal cardiomyocytes. SRF-GFP was expressed through a mammalian expression vector transfected into neonatal cadiomyocytes. TAT-SRF displayed same cellular localization as SRF-GFP and endogenous SRF. FIG. 15B shows western blot analysis of cardiomyocytes incubated with TAT-SRF at increasing concentrations. Anti-SRF recognizes both endogenous SRF and TAT-SRF, which have similar molecular weight. In FIG. 15C, SRF245A/254A mutant was resistant to caspase 3 cleavage. Purified TAT-SRF and TAT245A/254A were incubated with recombinant caspase 3 in vitro and only TAT-SRF was cleaved by caspase 3.

FIG. 16 shows real-time RT-PCR analysis of p21. RNA samples were prepared from ROCK1^{-/-} and control hearts after three-week aortic banding (n=3-4 for each group). Quantitative RT-PCR analysis was performed using the ABI Prism 7700 sequence detection system (Perkin Elmer). The levels of the transcripts were normalized to that of GAPDH. *P<0.05 vs. control.

FIG. 17 provides an exemplary diagram of the design of study of one embodiment of the invention.

FIG. 18 provides an exemplary diagram of the design of study of one embodiment of the invention.

FIG. 19 shows real-time RT-PCR analysis of ROCK1 and ROCK2 expression in pressure overload-induced hypertrophic hearts. RNA samples were prepared from control hearts after three-week aortic banding (n=3-4 for each group). Quantitative RT-PCR analysis-using specific oligonucleotide sets was performed. The levels of the transcripts were normalized to that of GAPDH. *P<0.05 vs. sham-operated mice.

FIG. 20 provides an exemplary strategy for generating conditional ROCK1 knockout mice. In the targeting vector, PGK-Neo is flanked by Frt sites (diamonds) and the gene segment containing the exon 8 is flanked by loxP sites (arrows). The critical ATP-binding catalytic lysine is located at residue 105. After homologous recombination, the Neo cassette is removed through Flp excision. Deletion of exon 8 after Cre recombination results in a frame-shift mutation from residue 137. H, HindIII; B, BamHI; X, XbaI.

FIG. 21 provides an exemplary strategy for generation of D1113A knockin mutation mice. Top: schematic representation of the ROCK1 protein structure. The box between the coiled-coil and PH domains represents the exon containing the caspase 3 cleavage site. Second: genomic DNA structure with the relevant restriction enzymes sites. Third: targeting vector contains the D1113A mutation resistant to caspase 3 cleavage, a PGK-Neo cassette with loxP sequence (arrows) at both sides (placed within the intron downstream of exon 30), and thymidine kinase gene (TK). Fourth: targeted allele after homologous recombination. The PGK-Neo cassette is excised upon crossing with the mice expressing germ line Cre recombinase (EIIa-cre). Bottom: the final targeted allele contains the mutated caspase 3 cleavage site and one loxP site within the downstream intron.

FIGS. 22A-22D show regulation of PTEN and AKT by ROCK-1. In FIG. 22A, cells were transfected with ROCKΔ1 and its kinase mutant KD. Cell lysates were analyzed for phospho-AKT (pAKT) by Western blot. In FIG. 22B, PTEN activities were assessed by Malachite green assay after cells were transfected with full length of ROCK-1 and its mutants. In FIG. 22C, pAKT and PTEN were detected by Western blot after cells were treated by PTEN-specific siRNA. Densitometry analysis for pAKT was shown in the lower panel after normalization by actin expression. In FIG. 22D, in co-transfection with PTEN-specific siRNA and ROCKΔ1, PTEN and pAKT levels were analyzed by Western blot. The expression levels were normalized to actin and shown in the lower panel. All experiments were conducted in human HEK cells. Δ1: active Rho kinase ROCK-1. KD: kinase deficient mutant ROCK-1. a.u.: artificial unit. NS-siRNA: non-specific siRNA.

FIGS. 23A-23D show blocked ROCK-1 expression prevented cardiomyocytes from apoptosis. In FIG. 23A, a specific siRNA significantly knocked down ROCK-1 expression without interrupting ROCK-2 expression (top left panel). Application of this siRNA inhibited the caspase 3 activation induced by ceramide (top right panel). In FIG. 23B, fluorescent staining showed that pre-treatment with the siRNA protected cardiomyocytes from ceramide-induced apoptosis. In FIG. 23C, a representative ROCK-1^{-/-} mouse myocardium image showing a TUNEL-positive cardiac myocyte revealed by TUNEL green staining after one week (1 W) aortic banding. In FIG. 23D, there is comparison of TUNEL-positive myocytes in left ventricle myocardium from wild type and ROCK-1^{-/-} mice after 1W aortic banding or surgical sham. Mouse number n=5 for each group. NS: non-specific; Red: phalloidin staining for F-actin; Blue: DAPI nucleus staining.

FIG. 24 shows exemplary proposal mechanisms involved in the cleavage activation of ROCK-1 and cardiac dysfunction.

FIGS. 25A-25D describe an exemplary luminescent kinase assay for identifying inhibitors of ROCK1.

### DETAILED DESCRIPTION OF THE INVENTION

I. Definitions

The term “a” or “an” as used herein in the specification may mean one or more. As used herein in the claim(s), when used in conjunction with the word “comprising”, the words “a” or “an” may mean one or more than one. As used herein “another” may mean at least a second or more. Some embodiments of the invention may consist of or consist essentially of one or more elements, method steps, and/or methods of the invention. It is contemplated that any method or composition described herein can be implemented with respect to any other method or composition described herein.

As used herein, the term “cardiac failure” refers to a clinical syndrome in which heart disease comprises reduction in cardiac output, increase in venous pressures, and is accompanied by molecular abnormalities that cause progressive deterioration of the failing heart and premature myocardial cell death.

The terms “cardiovascular disease” or “cardiac disease” as used herein is defined as a medical condition related to the cardiovascular (heart) or circulatory system (blood vessels). Cardiovascular disease includes, but is not limited to, diseases and/or disorders of the pericardium (i.e., pericardium), heart valves (i.e., incompetent valves, stenosed valves, rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). Yet further, one skill in the art recognizes that cardiovascular diseases can result from congenital defects, genetic defects, environmental influences (i.e., dietary influences, lifestyle, stress, etc.), and other defects or influences, and combinations thereof. In a specific embodiment, cardiac disease comprises failure of the heart.

The term “cardiovascular tissue” as used herein is defined as heart tissue and/or blood vessel tissue.

As used herein, the term “coronary artery disease” (CAD) refers to a type of cardiovascular disease. CAD is caused by gradual blockage of the coronary arteries. One of skill in the art realizes that in coronary artery disease, atherosclerosis (commonly referred to as “hardening of the arteries”) causes thick patches of fatty tissue to form on the inside of the walls of the coronary arteries. These patches are called plaque. As the plaque thickens, the artery narrows and blood flow decreases, which results in a decrease in oxygen to the myocardium. This decrease in blood flow precipitates a series of consequences for the myocardium. For example, interruption in blood flow to the myocardium results in an “infarct” (myocardial infarction), which is commonly known as a heart attack.

As used herein, the term “damaged myocardium” refers to myocardial cells that have been exposed to ischemic conditions. These ischemic conditions may be caused by a myocardial infarction, or other cardiovascular disease or related complaint. The lack of oxygen causes the death of the cells in the surrounding area, leaving an infarct, which eventually scars.

The term “fibrosis” as used herein refers to formation of fibrous tissue in the lining and the muscle of the heart.

As used herein, the term “infarct” or “myocardial infarction (MI)” refers to an interruption in blood flow to the myocardium. Thus, one of skill in the art refers to MI as death of cardiac muscle cells resulting from inadequate blood supply.

As used herein, the term “ischemic heart disease” refers to a lack of oxygen due to inadequate perfusion or blood supply. Ischemic heart disease is a condition having diverse etiologies. One specific etiology of ischemic heart disease is the consequence of atherosclerosis of the coronary arteries.

As used herein, the term “myocardium” refers to the muscle of the heart.

As used herein, the term “pharmaceutically acceptable carrier” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the vectors or cells of the present invention, its use in therapeutic compositions is contemplated. Supplementary active ingredients also can be incorporated into the compositions.

As used herein, the term “selectively inhibiting” refers to the preferential inhibition of ROCK 1 instead of other molecules, such as other Rho kinase-related molecules, including ROCK2. The selective inhibition may be complete, such as the agent being ineffective against ROCK2, including having no detectable effect on ROCK2. In alternative embodiments, the selective inhibition permits a minor amount of inhibition of ROCK2, although significantly reduced compared to the inhibition of ROCK1. In specific embodiments, ROCK1 inhibition is about 5-fold greater than ROCK 2, is about 10-fold greater than ROCK2, is about 50-fold greater than ROCK2, is about 100-fold greater than ROCK2, is about 500-fold greater than ROCK2, is about 1000-fold greater than ROCK2, is about 10,000-fold greater than ROCK2, and so forth.

The term “therapeutically effective amount” as used herein refers to an amount that results in an improvement or remediation of the disease, disorder, or symptoms of the disease or condition.

The term “treating” and “treatment” as used herein refers to administering to a subject a therapeutically effective amount of a the composition so that the subject has an improvement in the disease. The improvement is any improvement or remediation of the symptoms. The improvement is an observable or measurable improvement. Thus, one of skill in the art realizes that a treatment may improve the disease condition, but may not be a complete cure for the disease.

II. The Present Invention

The present invention generally concerns preventing apoptosis and/or fibrosis in tissue, particularly cardiac tissue, by inhibiting Rho kinase. Specifically, it concerns cardiac therapy and/or diagnosis generally related to Rho kinase. In specific embodiments, it concerns selective inhibition of ROCK1 in the absence of inhibiting ROCK2 for treatment and/or prevention of cardiac failure. In particular embodiments, cross-kinase inhibition is undesirable for a variety of reasons, and thus the invention concerns specific inhibition of ROCK1 to the exclusion of inhibition of ROCK2. In particular embodiments, the selective inhibition of ROCK1 occurs in all tissues in which ROCK1 and ROCK2 are expressed, or it may selectively inhibit ROCK1 in only certain tissues, such as only in cardiac tissue, liver tissue, kidney tissue, lung tissue, or vasculature tissue, for example. In specific embodiments, there is specific inhibition of ROCK1 and not ROCK2 that inhibits apoptosis and/or fibrosis while maintaining the adaptive response of cardiomyocyte enlargement. This is in direct contrast to other Rho kinase inhibitors, such as Y27632 and fasudil, for example.

Heart failure develops at the end-stage of any heart disease with chronic mechanical overload. A loss of cardiomyocytes by overload-induced apoptosis, in one embodiment of the invention, results in the progressive character of the disease. Two aspects of cardiac apoptosis are related to the present invention: 1) signaling pathways activated in response to pressure overload trigger myocyte apoptosis; and 2) contributory roles to the transition from compensated hypertrophy to heart failure. Although the concept for a pathological role of caspase 3 activation in heart failing has been widely accepted, the extent of its contribution to the development of heart failure, as well as the mechanisms mediating its effects on myocardial structure and function changes, remain poorly understood. A number of molecules have recently been identified as direct substrates of caspase 3 in failing cardiomyocytes, but their contributory roles to the depressed contractility and cell loss are only speculative.

Among the signaling pathways activated in response to pressure overload, those signaling pathways involved RhoA, ROCK1 and SRF are of particular interest. Besides their numerous functions in cellular biology, their roles in mediating the mechanical signals and in cardiomyocyte apoptosis are characterized herein. In a specific embodiment, ROCK1 activation in response to pressure overload potentiates the transition to heart failure. The present inventors have also established a correlative relationship between caspase 3 activation and changes in the expression and/or activity of ROCK1 and SRF in apoptotic cardiomyocytes and in human failing hearts. Their contributory roles and mechanistic insights in the pathogenesis of chronic heart failure using appropriate exemplary in vitro and in vivo experimental systems are described herein.

In particular, pathological cardiac hypertrophy in response to pressure overload is initially a compensatory response but eventually leads to decompensation resulting in heart failure or sudden death. Identification of signaling pathways regulating hypertrophic growth, myocyte contractility and survival provides useful information for therapy aimed at preventing or retarding the development of heart failure. Through a loss-of-function approach, the present inventors have demonstrated that ROCK1 is an important mediator of hypertrophic and apoptotic signals under pressure overload condition. They have also identified ROCK1 and SRF as direct substrates of activated caspase 3 in human failing hearts and in apoptotic cardiomyocytes. The present invention determines the contributory roles of ROCK1 and its activation by caspase 3 cleavage in the initiation and progression of heart failure. As a part of the invention, transgenic mouse models that mimic the pathological processes observed in failing human hearts in order to understand the underlying mechanisms are provided.

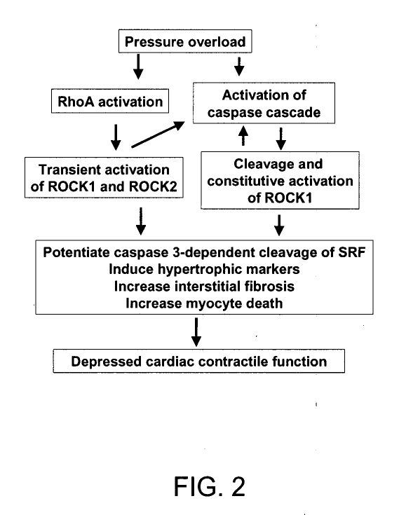

FIG. 2 illustrates an exemplary schematic for the present invention. Pressure overload activates RhoA signaling pathway, which in turn transiently activates ROCK1 as well as ROCK2. Activation of apoptotic machinery by pressure overload leads to caspase 3 activation, which constitutively activates ROCK1 (but not ROCK2) through a RhoA-independent mechanism. Activation of ROCK1 by RhoA or by caspase 3 in turn activates caspase cascade and/or potentiate caspase 3-dependent cleavage of other caspase 3 substrates including SRF. Activation of ROCK1 in response to pressure overload plays an important role in the pathological changes of gene expression profiles, and in pathological remodeling including increased myocyte death and interstitial fibrosis, resulting in depressed cardiac contractile function. Activation of ROCK1 may also directly regulate myocyte contractility. The contributory role of transient activation of ROCK1 by RhoA versus constitutive activation of ROCK1 by caspase 3 to the development of heart failure will be evaluated in genetically modified mouse models.

Role of Rho kinase in cardiac hypertrophy and heart failure. Rho kinase is a downstream mediator of RhoA, and is believed to play a critical role in mediating the effects of RhoA on stress fiber formation, smooth muscle contraction, cell adhesion, membrane ruffling and cell motility (Amano et al., 2000; Riento and Ridley, 2003). The Rho kinase family contains two members: ROCK1 (p160ROCK) and its close relative ROCK2 (ROKα) (Matsui et al., 1996; Ishizaki et al., 1996; Nakagawa et al., 1996). The best characterized in vivo downstream mediators of Rho kinase are myosin light chain phosphatase, myosin light chain (Amano et al., 1997; Kimura et al., 1996; Leung et al., 1996) and LIM kinase (Maekawa et al., 1999; Arber et al., 1998).

Rho kinase inhibition in cultured cardiomyocytes (by a specific pharmacological Rho kinase inhibitor, Y27632 or by dominant-negative mutants of Rho kinase, for example) indicates that Rho kinase mediates part of the effect of RhoA on myofiber assembly and hypertrophic gene expression induced by phenylephrine, endothelin-1 or activated RhoA (Hoshijima et al., 1998; Kawahara et al., 1999). Rho kinase expression and activity are also up-regulated in the failing heart of Dahl salt-sensitive hypertensive rats (Kobayashi et al., 2002; Satoh et al., 2003), and in the hypertrophic heart of angiotensin II-infused rats (Higashi et al., 2003). Administration of Y27632 to Dahl salt-sensitive hypertensive rats, leads to the regression of cardiac hypertrophy and decreased pathological remodeling (Kobayashi et al., 2002; Satoh et al., 2003). Administration of fasudil, another chemical inhibitor of Rho kinase, suppresses angiotensin II-induced coronary vascular hypertrophy, endothelial dysfunction and cardiomyocyte hypertrophy (Higashi et al., 2003). However, in these studies, the chemical inhibitors of Rho kinase inhibit the activity of both Rho kinase isoforms, which may have differential functional activities in the pathogenesis of cardiac dysfunction and remodeling under pressure overload. The ROCK1-deficient mice described herein provide a unique model to address specific roles of this Rho kinase isoform in cardiac hypertrophy and heart failure.

Role of Rho kinases in apoptosis of non-cardiac and cardiac cells. The majority of studies investigating a potential role of Rho kinase in apoptosis have not been performed in cardiac cells. Inhibition of the Rho kinase pathway has different effects on survival and apoptosis depending on cell type and on apoptotic stimuli. The anti-apoptotic effect of Rho kinase has been observed in cultured airway epithelial cells (Moore et al., 2003), vascular smooth muscle (Matsumoto et al., 2003) and human umbilical vein endothelial cells (Li et al., 2002), hepatic stellate cells (Ikeda et al., 2003) and also rat neonatal cardiomyocytes (Ogata et al., 2002). In specific embodiments, this survival effect of Rho kinase is mediated by its role in maintaining the integrity of actin cytoskeletal structure.

In another embodiment, the apoptotic effect of Rho kinase has been observed in an erythroblastic cell line (TF-1) treated with phorbol ester (Lai et al., 2002; Lai et al., 2003), bovine pulmonary endothelial cells treated with TNFα (petrache et al., 2003). This apoptotic effect of Rho kinase is believed to be mediated via regulation of actin cytoskeletal rearrangement, which in turn induces the activation of the caspase cascade (possibly via the assembly of the death-inducing signaling complex) (Lai et al., 2003; Petrache et al., 2003). Recently, ROCK1 was shown to be a direct substrate of caspase 3 in NIH3T3 fibroblasts (Coleman et al., 2001), HeLa cells (Ueda et al., 2001), haematopoietic cell lines and epithelial cell lines (Sebbagh et al., 2001). Cleaved ROCK1 regulates actin cytoskeleton during apoptosis and is responsible for bleb formation in apoptotic cells (Coleman et al., 2001; Ueda et al., 2001; Sebbagh et al., 2001). These observations indicate that Rho kinase has multiple roles in apoptosis.

The present inventors have observed that ROCK1 is cleaved by caspase 3 in human failing hearts and in apoptotic cardiomyocytes. In addition, the cleaved ROCK1 fragment is sufficient to induce activation of caspase 3 in cultured cardiomyocytes, suggesting that caspase 3-dependent cleavage and activation of ROCK1 may play an important role in myocyte apoptosis.

Role of SRF in cardiac hypertrophy and heart failure. SRF is a member of an ancient family of DNA-binding proteins, which contain a highly conserved DNA binding/dimerization domain of 90 amino acids, termed the MADS box (Norman et al., 1988). A large number of cardiac and smooth muscle genes contain serum response elements in their promoter regions (Lee and Schwartz, 1992; Li et al., 1997). Transgenic mice with cardiac specific overexpression of wild-type or a dominant negative mutant SRF develop cardiac hypertrophy or dilated heart failure, respectively (Zhang et al., 2001; Zhang et al., 2001). SRF mutants derived from alternative splicing or truncation of the C-terminal transactivation domain act as a dominant negative for endogenous SRF (Belaguli et al., 1999). Two recent studies in non-cardiac cell culture systems have shown that caspase 3 activation in apoptotic cells leads to SRF cleavage (Drewett et al., 2001; Betolotto et al., 2000), and that the expression of an SRF mutant, which can not be cleaved by caspase 3, significantly suppresses apoptosis (Drewett et al., 2001). The present inventors determined that SRF is also a direct target of caspase 3 in failing heart and a 32 kDa SRF cleavage product acts as a dominant negative transcription factor, indicating that caspase 3-dependent cleavage of SRF leads to the alteration of the expression of many cardiac genes.

Role of Rho kinase in regulating SRF transcriptional activity. The inventors determined that Rho kinase directly phosphorylates SRF on its MADS box, and this phosphorylation selectively inhibits SRF myogenic gene targets in C2C12 myoblasts and embryonic stem cells. Consistent with this finding, precocious expression of cardiac α-actin was observed (an early cardiac differentiation marker) in early chick embryos treated with Rho kinase inhibitor, Y27632, indicating that Rho kinase inhibits cardiomyocyte differentiation and SRF myogenic gene targets in precardiac cells (Wei et al., 2001). Recent in vitro data indicate a novel role for SRF phosphorylation by Rho kinase: in specific embodiments, this phosphorylation facilitates SRF cleavage by caspase 3, contributing to the down-regulation of SRF expression in failing heart.

III. ROCK

ROCK1 is a RhoA-binding protein with Ser/Thr protein kinase activity and is 1358 amino acids in length. The polypeptide includes a catalytic kinase domain at the N-terminus, which is about 300 amino acids in length and comprises the conserved motifs characteristic of Ser/Thr kinases; the kinase domain is also involved in binding to RhoE, which is a negative regulator of ROCK activity. In addition, the C-terminus of ROCK1 has several functional domains, including a Rho-binding domain within a flexible coiled-coil region, a pleckstrin homology (PH) domain, and a cysteine-rich domain. In some embodiments, the PH domain is likely necessary for regulation by interacting with lipid messengers, for example, arachidonic acid.

Exemplary ROCK inhibitors include Y-27632 and fasudil, which bind to the kinase domain to inhibit its enzymatic activity in an ATP-competitive mechanism. Negative regulators of ROCK activation include small GTP-binding proteins such as Gem, RhoE, and Rad, which can attenuate ROCK activity. Autoinhibitory activity of ROCK is demonstrated upon interaction of the carboxyl terminus with the kinase daomain to reduce kinase activity. The Rho-binding domain, which is about 80 amino acids in length and is required for interaction with activated RhoA, comprises considerable sequence similarity to domains present in some Rho binding proteins. Additional ROCK inhibitors include WO 01/56988; WO 02/100833; WO 03/059913; WO 02/076976; WO 04/029045; WO 03/064397; WO 04/039796; WO 05/003101; WO 02/085909; WO 03/082808; WO 03/080610; WO 04/112719; WO 03/062225; and WO 03/062227, for example. In some of these cases, motifs in the inhibitors include an indazole core; a 2-aminopyridine/pyrimidine core; a 9-deazaguanine deriviative; benzamide-comprising; aminofurazan-comprising; and/or a combination thereof.

The two isoforms of ROCK include ROCK1 (which may also be referred to as ROK-β or p160ROCK) and ROCKII (which may also be referred to as ROK-α or Rho-kinase). The two isoforms have 65% sequence similarity overall, and the kinase domains comprise 92% sequence identity. Although both isoforms are ubiquitously expressed in tissues, there are differing intensities in certain tissues. In specific embodiments of the invention, ROCK1 is targeted instead of ROCK 2, and the agent for such is an inhibitor that binds to an allosteric site, for example.

IV. Treatment of Cardiovascular/Cardiac Disease

Cardiovascular/cardiac diseases and/or disorders include, but are not limited to, diseases and/or disorders of the pericardium (i.e., pericardium), heart valves (i.e., incompetent valves, stenosed valves, Rheumatic heart disease, mitral valve prolapse, aortic regurgitation), myocardium (coronary artery disease, myocardial infarction, heart failure, ischemic heart disease, angina) blood vessels (i.e., arteriosclerosis, aneurysm) or veins (i.e., varicose veins, hemorrhoids). In specific embodiments, the cardiovascular disease includes, but is not limited to, coronary artery diseases (i.e., arteriosclerosis, atherosclerosis, and other diseases of the arteries, arterioles and capillaries or related complaint), myocardial infarction and ischemic heart disease.

V. Combined Cardiac Disease Treatments

In order to increase the effectiveness of the compositions and/or methods described herein, it may be desirable to combine these compositions and methods of the invention with a known agent effective in the treatment of cardiac disease or disorder. In some embodiments, it is contemplated that a conventional therapy or agent, including but not limited to, a pharmacological therapeutic agent, a surgical therapeutic agent (e.g., a surgical procedure), a device, or a combination thereof, may be combined with the agent of the invention (such as a nucleic acid, peptide, polyeptide, PH domain, caspase inhibitor(s) and/or uncleavable Rho kinase). In a non-limiting example, a therapeutic benefit comprises repair of myocardium or vascular tissue or reduced restenosis following vascular or cardiovascular intervention, such as occurs during a medical or surgical procedure, for example.

This process may involve contacting the cell(s) with an agent(s) and the caspase inhibitor(s) and/or uncleavable Rho kinase of the present invention at substantially the same time or within a period of time wherein separate administration of the caspase inhibitor(s) and/or uncleavable Rho kinase and an agent to a cell, tissue or organism produces a desired therapeutic benefit. The terms “contacted” and “exposed,” when applied to a cell, tissue or organism, are used herein to describe the process by which the caspase inhibitor(s) and/or uncleavable Rho kinase and/or therapeutic agent(s) are delivered to a target cell, tissue or organism or are placed in direct juxtaposition with the target cell, tissue or organism. The cell, tissue or organism may be contacted (e.g., by administration) with a single composition or pharmacological formulation that comprises both a caspase inhibitor(s) and/or uncleavable Rho kinase and one or more agents, or by contacting the cell with two or more distinct compositions or formulations, wherein one composition includes a caspase inhibitor(s) and/or uncleavable Rho kinase and the other includes one or more agents.

The treatment may precede, be concurrent with and/or follow the other agent(s) by intervals ranging from minutes to weeks. In embodiments where the caspase inhibitor(s) and/or uncleavable Rho kinase, and other agent(s) are applied separately to a cell, tissue or organism, one would generally ensure that a significant period of time did not expire between the time of each delivery, such that the caspase inhibitor(s) and/or uncleavable Rho kinase and agent(s) would still be able to exert an advantageously combined effect on the cell, tissue or organism. For example, in such instances, it is contemplated that one may contact the cell, tissue or organism with two, three, four or more modalities substantially simultaneously (i.e. within less than about a minute) as the caspase inhibitor(s) and/or uncleavable Rho kinase. In other aspects, one or more agents may be administered within of from substantially simultaneously, about minutes to hours to days to weeks and any range derivable therein, prior to and/or after administering the smooth cells or a tissue derived therefrom.

Administration of the caspase inhibitor(s) and/or uncleavable Rho kinase composition to a cell, tissue or organism may follow general protocols for the administration of vascular or cardiovascular therapeutics, taking into account the toxicity, if any. It is expected that the treatment cycles would be repeated as necessary. In particular embodiments, it is contemplated that various additional agents may be applied in any combination with the present invention.

A. Pharmacological Therapeutic Agents

Pharmacological therapeutic agents and methods of administration, dosages, etc., are well-known to those of skill in the art (see for example, the “Physicians Desk Reference”, Goodman & Gilman's “The Pharmacological Basis of Therapeutics”, “Remington's Pharmaceutical Sciences”, and “The Merck Index, Eleventh Edition”, incorporated herein by reference in relevant parts), and may be combined with the invention in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Non-limiting examples of a pharmacological therapeutic agent that may be used in the present invention include an antihyperlipoproteinemic agent, an antiarteriosclerotic agent, an antithrombotic/fibrinolytic agent, a blood coagulant, an antiarrhythmic agent, an antihypertensive agent, a vasopressor, a treatment agent for congestive heart failure, or a combination thereof.

B. Surgical Therapeutic Agents

In certain aspects, a therapeutic agent may comprise a surgery of some type, which includes, for example, preventative, diagnostic or staging curative and/or palliative surgery. Surgery, and in particular, a curative surgery, may be used in conjunction with other therapies, such as the present invention and one or more other agents.

Such surgical therapeutic agents for vascular and cardiovascular diseases and disorders are well known to those of skill in the art, and may comprise, but are not limited to, performing surgery on an organism, providing a cardiovascular mechanical prostheses, angioplasty, coronary artery reperfusion, catheter ablation, providing an implantable cardioverter defibrillator to the subject, mechanical circulatory support or a combination thereof. Non-limiting examples of a mechanical circulatory support that may be used in the present invention comprise an intra-aortic balloon counterpulsation, left ventricular assist device or combination thereof.

Further treatment of the area of surgery may be accomplished by perfusion, direct injection, systemic injection or local application of the area with at least one additional therapeutic agent (e.g., a caspase inhibitor(s) and/or uncleavable SRF, a pharmacological therapeutic agent, and so forth), as would be known to one of skill in the art or described herein.

VI. Kits

Any of the compositions described herein may be comprised in a kit. In a non-limiting example, a selective inhibitor of ROCK1 and/or additional agent may be comprised in a kit. The kits may thus comprise, in suitable container means, the inhibitor and/or an additional agent of the present invention. In specific embodiments, the kit comprises a ROCK1 inhibitor that does not also inhibit ROCK2.

The kits may comprise a suitably aliquoted inhibitor(s) and/or additional agent compositions of the present invention, whether labeled or unlabeled, as may be used to prepare a standard curve for a detection assay. The components of the kits may be packaged either in aqueous media or in lyophilized form. The container means of the kits may generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the inhibitor and/or the pharmacological composition of the present invention, lipid, additional agent, and any other reagent containers in close confinement for commercial sale. Such containers may include injection or blow-molded plastic containers into which the desired vials are retained.

Therapeutic kits of the present invention are kits comprising one or more ROCK1-selective inhibitors. Such kits will generally contain, in suitable container means, a pharmaceutically acceptable formulation of a selective inhibitor(s) of ROCK1. The kit may have a single container means, and/or it may have distinct container means for each compound.

When the components of the kit are provided in one and/or more liquid solutions, the liquid solution is an aqueous solution, with a sterile aqueous solution being particularly preferred. The stem cell compositions may also be formulated into a syringeable composition. In which case, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means.

The container means will generally include at least one vial, test tube, flask, bottle, syringe and/or other container means, into which the stem cells are placed, preferably, suitably allocated. The kits may also comprise a second container means for containing a sterile, pharmaceutically acceptable buffer and/or other diluent.

The kits of the present invention will also typically include a means for containing the vials in close confinement for commercial sale, such as, e.g., injection and/or blow-molded plastic containers into which the desired vials are retained.

Irrespective of the number and/or type of containers, the kits of the invention may also comprise, and/or be packaged with, an instrument for assisting with the injection/administration and/or placement of the ultimate the stem cell composition within the body of an animal. Such an instrument may be a syringe, pipette, forceps, and/or any such medically approved delivery vehicle.

VII. Definitions and Techniques Affecting Gene Products and Genes

A. Rho kinase Gene Products and Genes

In this patent, the terms “Rho kinase gene product” and “Rho kinase ” refer to proteins and polypeptides having amino acid sequences that are substantially identical to the native Rho kinase amino acid sequences (or RNA, if applicable) or that are biologically active, in that they are capable of performing functional activities similar to an endogenous Rho kinase and/or cross-reacting with anti-Rho kinase antibody raised against Rho kinase. In analogous embodiments, “Rho kinase gene product,” and “Rho kinase” are referred to herein.

An example of a Rho kinase polypeptide sequence, followed by its National Center for Biotechnology's GenBank database Accession No. includes the exemplary ROCK1 polypeptide comprising SEQ ID NO:1 (NP_{—}005397), or a functionally similar fragment thereof. A skilled artisan can employ these exemplary sequences to identify peptides or nucleic acids suitable for therapeutic applications.

The term “Rho kinase gene product” includes analogs of the respective molecules that exhibit at least some biological activity in common with their native counterparts. Such analogs include, but are not limited to, truncated polypeptides and polypeptides having fewer amino acids than the native polypeptide. Furthermore, those skilled in the art of mutagenesis will appreciate that homologs to the mouse Rho kinase polynucleotide, including human homologs, which homologs are as yet undisclosed or undiscovered, may be used in the methods and compositions disclosed herein.

The term “Rho kinase gene” “Rho kinase polynucleotide” or “Rho kinase nucleic acid” refers to any DNA sequence that is substantially identical to a DNA sequence encoding an Rho kinase gene product as defined above. The term also refers to RNA or antisense sequences compatible with such DNA sequences. An “Rho kinase gene or Rho kinase polynucleotide” may also comprise any combination of associated control sequences. In a specific embodiment of the present invention, a Rho kinase polynucleotide including the exemplary ROCK1 polynucleotide of SEQ ID NO:2 (NM_{—}005406), or a functionally similar fragment thereof, is utilized. In specific embodiments, this exemplary ROCK1 polynucleotide or a fragment thereof is employed as an inhibitor of ROCK1 expression. For example, the inhibitor may comprise RNAi, siRNA, antisense ROCK1, and so forth.

Thus, nucleic acid compositions encoding ROCK1 are herein provided and are also available to a skilled artisan at accessible databases, including the National Center for Biotechnology Information's GenBank database and/or commercially available databases, such as from Celera Genomics, Inc. (Rockville, Md.). Also included are splice variants that encode different forms of the protein, if applicable. The nucleic acid sequences may be naturally occurring or synthetic.

As used herein, the terms “ROCK1 nucleic acid sequence,” “ROCK1 polynucleotide,” and “ROCK1 gene” refer to nucleic acids provided herein, homologs thereof, and sequences having substantial similarity and function, respectively. A skilled artisan recognizes that the sequences are within the scope of the present invention if they encode a product which, facilitates diagnosis of cardiac failure and/or provides cardiac disease therapy, and furthermore knows how to obtain such sequences, as is standard in the art.

The term “substantially identical”, when used to define either a ROCK1 amino acid sequence or ROCK1 polynucleotide sequence, means that a particular subject sequence, for example, a mutant sequence, varies from the sequence of natural ROCK1 by one or more substitutions, deletions, or additions, the net effect of which is to retain at least some biological activity of the ROCK1 protein, respectively. Alternatively, DNA analog sequences are “substantially identical” to specific DNA sequences disclosed herein if: (a) the DNA analog sequence is derived from coding regions of the natural ROCK1 gene; or (b) the DNA analog sequence is capable of hybridization of DNA sequences of (a) under moderately stringent conditions and which encode biologically active ROCK1; or (c) DNA sequences which are degenerative as a result of the genetic code to the DNA analog sequences defined in (a) or (b). Substantially identical analog proteins will be greater than about 80% similar to the corresponding sequence of the native protein. Sequences having lesser degrees of similarity but comparable biological activity are considered to be equivalents. In determining polynucleotide sequences, all subject polynucleotide sequences capable of encoding substantially similar amino acid sequences are considered to be substantially similar to a reference polynucleotide sequence, regardless of differences in codon sequence.

1. Percent Similarity

Percent similarity may be determined, for example, by comparing sequence information using the GAP computer program, available from the University of Wisconsin Geneticist Computer Group. The GAP program utilizes the alignment method of Needleman et al., 1970, as revised by Smith et al., 1981. Briefly, the GAP program defines similarity as the number of aligned symbols (i.e. nucleotides or amino acids) which are similar, divided by the total number of symbols in the shorter of the two sequences. The preferred default parameters for the GAP program include (1) a unitary comparison matrix (containing a value of 1 for identities and 0 for non-identities) of nucleotides and the weighted comparison matrix of Gribskov et al., 1986, (2) a penalty of 3.0 for each gap and an additional 0.01 penalty for each symbol and each gap; and (3) no penalty for end gaps.

2. Polynucleotide Sequences

In certain embodiments, the invention concerns the use of Rho kinase genes and gene products, such as the Rho kinase that includes a sequence which is essentially that of the known Rho kinase gene, or the corresponding protein, respectively. The term “a sequence essentially as Rho kinase ” means that the sequence substantially corresponds to a portion of the ROCK1 gene, respectively, and has relatively few bases or amino acids (whether DNA or protein) that are not identical to those of ROCK1 (or a biologically functional equivalent thereof, when referring to proteins), respectively. The term “biologically functional equivalent” is well understood in the art and is further defined in detail herein. Accordingly, sequences that have between about 70% and about 80%; or more preferably, between about 81% and about 90%; or even more preferably, between about 91% and about 99%; of amino acids which are identical or functionally equivalent to the amino acids of ROCK1 will be sequences which are “essentially the same”.

ROCK1 genes that have functionally equivalent codons, respectively, are also covered by the invention. The term “functionally equivalent codon” is used herein to refer to codons that encode the same amino acid, such as the six codons for arginine or serine, and also refers to codons that encode biologically equivalent amino acids (Table 1).

TABLE 1
FUNCTIONALLY EQUIVALENT CODONS.

| | |
|---|---|
| Amino Acids | Codons |

| | | | | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic Acid | Asp | D | GAC | GAU | | | | |
| Glutamic Acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCU | | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

It will also be understood that amino acid and polynucleotide sequences may include additional residues, such as additional N— or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to polynucleotide sequences that may, for example, include various non-coding sequences flanking either of the 5′ or 3′ portions of the coding region or may include various internal sequences, i.e., introns, which are known to occur within genes.

In certain embodiments, the invention concerns the use of uncleavable Rho kinase polynucleotide sequences, truncated Rho kinase polynucleotide sequences or polynucleotide sequences that encode a Rho kinase polypeptide, respectively, with less amino acids than native Rho kinase. The present invention also encompasses the use of DNA segments that are complementary, or essentially complementary, to the sequences set forth in the specification. Polynucleotide sequences that are “complementary” are those that are capable of base-pairing according to the standard Watson-Crick complimentarily rules. As used herein, the term “complementary sequences” means polynucleotide sequences which are substantially complementary, as may be assessed by the same nucleotide comparison set forth above, or as defined as being capable of hybridizing to the polynucleotide segment in question under relatively stringent conditions such as those described herein.

3. Biologically Functional Equivalents

As mentioned above, modification and changes may be made in the structure of Rho kinase and still obtain a molecule having like or otherwise desirable characteristics, respectively. For example, certain amino acids may be substituted for other amino acids in a protein structure without appreciable loss of activity for upregulating expression of smooth muscle-specific polynucleotides. Since it is the interactive capacity and nature of a protein that defines that protein's biological functional activity, certain amino acid sequence substitutions and/or deletions can be made in a protein sequence (or, of course, its underlying DNA coding sequence) and nevertheless obtain a protein with like or even countervailing properties (e.g., antagonistic v. agonistic). It is thus contemplated by the inventors that various changes may be made in the sequence of the Rho kinase proteins or peptides (or underlying DNA) without appreciable loss of their biological utility or activity, respectively. Included in such changes are truncated-Rho kinase polypeptides and Rho kinase polypeptides having less amino acid residues than native Rho kinase.

It is also well understood by the skilled artisan that, inherent in the definition of a biologically functional equivalent protein or peptide, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted. Of course, a plurality of distinct proteins/peptides with different substitutions may easily be made and used in accordance with the invention.

It is also well understood that where certain residues are shown to be particularly important to the biological or structural properties of a protein or peptide, e.g., residues in active sites, such residues may not generally be exchanged. This is the case in the present invention, where any changes in Rho kinase that render the respective polypeptide incapable of preventing or delaying entry into mitosis following DNA damage would result in a loss of utility of the resulting peptide for the present invention.

Amino acid substitutions, such as those that might be employed in modifying Rho kinase are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ±2 is preferred, those that are within ±1 are particularly preferred, and those within ±0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0); threo (□0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid.

4. Sequence Modification Techniques

Modifications to the Rho kinase peptides may be carried out using techniques such as site-directed mutagenesis. Such modifications may comprise those directed to producing an uncleavable Rho kinase. In specific embodiments, an uncleavable SRF is defined as one lacking cleavability. Site-specific mutagenesis is a technique useful in the preparation of individual peptides, or biologically functional equivalent proteins or peptides, through specific mutagenesis of the underlying DNA. The technique further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Typically, a primer of about 17 to 25 nucleotides in length is preferred, with about 5 to 10 residues on both sides of the junction of the sequence being altered.

In general, the technique of site-specific mutagenesis is well known in the art as exemplified by publications (Adelman et al., 1983). As will be appreciated, the technique typically employs a phage vector that exists in both a single stranded and double stranded form. Typical vectors useful in site-directed mutagenesis include vectors such as the M13 phage (Messing et al., 1981). These phage are readily commercially available and their use is generally well known to those skilled in the art. Double stranded plasmids are also routinely employed in site directed mutagenesis which eliminates the step of transferring the gene of interest from a plasmid to a phage.

In general, site-directed mutagenesis in accordance herewith is performed by first obtaining a single-stranded vector or melting apart the two strands of a double stranded vector which includes within its sequence a DNA sequence which encodes at least the Rho kinase gene. An oligonucleotide primer bearing the desired mutated sequence is prepared, generally synthetically, for example by the method of Crea et al. (1978). This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected that include recombinant vectors bearing the mutated sequence arrangement.

The preparation of sequence variants of the selected gene using site-directed mutagenesis is provided as a means of producing potentially useful Rho kinase and is not meant to be limiting as there are other ways in which sequence variants of these peptides may be obtained. For example, recombinant vectors encoding the desired genes may be treated with mutagenic agents to obtain sequence variants (see, e.g., a method described by Eichenlaub, 1979) for the mutagenesis of plasmid DNA using hydroxylamine.

5. Antisense Constructs

In some cases, a gene is essential to the life of the cell, wherein its removal, such as by homologous replacement, results in the death of the cell. In other cases, a gene may have aberrant functions that cannot be overcome by replacement gene therapy, even where the “wild-type” molecule is expressed in amounts in excess of the mutant polypeptide. Antisense treatments are one way of addressing these situations. Antisense technology also may be used to “knock-out” function of ROCK1 and/or SRF for a therapeutic purpose and/or in the development of cell lines or transgenic mice for research, diagnostic and screening purposes, for example.

Antisense methodology takes advantage of the fact that nucleic acids tend to pair with “complementary” sequences. By complementary, it is meant that polynucleotides are those that are capable of base-pairing according to the standard Watson-Crick complimentarily rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs will include regions complementary to intron/exon splice junctions. Thus, it is proposed that a preferred embodiment includes an antisense construct with complimentarily to regions within 50-200 bases of an intron-exon splice junction. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences that are completely complementary will be sequences that are entirely complementary throughout their entire length and have no base mismatches. Other sequences with lower degrees of homology also are contemplated. For example, an antisense construct that has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

6. RNA Interference

RNA interference (RNAi) is the process of sequence-specific, post-transcriptional gene silencing in animals and plants, initiated by double-stranded RNA (dsRNA) that is homologous in sequence to the silenced gene. Elbashir et al. (2001a) demonstrated that 21- and 22-nt RNA fragments are the sequence-specific mediators of RNAi. In a specific embodiment, the short interfering RNAs (siRNAs) are generated by an RNase III-like processing reaction from long dsRNA. Chemically synthesized siRNA duplexes with overhanging 3′ ends mediate efficient target RNA cleavage in the lysate, and the cleavage site is located near the center of the region spanned by the guiding siRNA. Furthermore, the direction of dsRNA processing determines whether sense or antisense target RNA can be cleaved by the siRNA-protein complex. Also, Elbashir et al. (2001b) showed that 21-nucleotide siRNA duplexes specifically suppress expression of endogenous and heterologous genes in different mammalian cell lines, including human embryonic kidney (293) and HeLa cells.

Therefore, a skilled artisan recognizes that 21-nucleotide siRNA duplexes provide an effective tool for studying gene function in mammalian cells and are useful as gene-specific therapeutics.

7. Synthetic Polypeptides

The present invention also describes Rho kinase proteins and related peptides for use in various embodiments of the present invention. The Rho kinase polypeptide may have fewer amino acids than native Rho kinase. Relatively small peptides can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979), each incorporated herein by reference. Short peptide sequences, or libraries of overlapping peptides, usually from about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acids, which correspond to the selected regions described herein, can be readily synthesized and then screened in screening assays designed to identify reactive peptides. Alternatively, recombinant DNA technology may be employed wherein a nucleotide sequence which encodes a peptide of the invention is inserted into an expression vector, transformed or transfected into an appropriate host cell and cultivated under conditions suitable for expression.

8. Other Structural Equivalents

In addition to the Rho kinase peptidyl compounds described herein, the inventors also contemplate that other sterically similar compounds may be formulated to mimic the key portions of the peptide structure. Such compounds may be used in the same manner as the peptides of the invention and hence are also functional equivalents. The generation of a structural functional equivalent may be achieved by the techniques of modeling and chemical design known to those of skill in the art. It will be understood that all such sterically similar constructs fall within the scope of the present invention.

B. Expression Vectors

In certain aspects of the present invention it may be necessary to express the Rho kinase proteins and/or polypeptides. Throughout this application, the term “expression construct” is meant to include any type of genetic construct containing a polynucleotide coding for a gene product in which part or all of the polynucleotide encoding sequence is capable of being transcribed. The transcript may be translated into a protein, but it need not be. Thus, in certain embodiments, expression includes both transcription of a Rho kinase polynucleotide, respectively, and translation of the respective Rho kinase mRNA into an Rho kinase protein or polypeptide product, respectively. In other embodiments, expression only includes transcription of the polynucleotide encoding an Rho kinase or its complement. In some embodiments, the Rho kinase sequences are comprised on three or more separate vectors. In other embodiments, the Rho kinase sequences are comprised on one or two vectors.

A skilled artisan recognizes that if more than one vector is utilized, it is preferential to have nonidentical means, such as markers, to monitor uptake of the vector. Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art Examples of some markers include ampicillin, neomycin, kanamycin, tetracycline, and β-galactosidase.

In order for the construct to effect expression of at least a Rho kinase transcript, the polynucleotide encoding the Rho kinase polynucleotide, respectively, will be under the transcriptional control of a promoter. A “promoter” refers to a DNA sequence recognized by the synthetic machinery of the host cell, or introduced synthetic machinery, that is required to initiate the specific transcription of a gene. The phrase “under transcriptional control” means that the promoter is in the correct location in relation to the polynucleotide to control RNA polymerase initiation and expression of the polynucleotide. In specific embodiments, the promoter comprises a SRE or CArG box. In a specific embodiment, the promoter is SM22α promoter or SMA promoter

In a preferred embodiment the promoter is a synthetic myogenic promoter and hGH 3′ untranslated region is in the 3′ untranslated region. In a specific embodiment of the present invention there is utilized a synthetic promoter, termed SPc5-12 (Li et al., 1999) which contains a proximal serum response element (SRE) from skeletal α-actin, multiple MEF-2 sites, MEF-1 sites, and TEF-1 binding sites, and greatly exceeds the transcriptional potencies of natural myogenic promoters. Other elements, including trans-acting factor binding sites and enhancers may be used in accordance with this embodiment of the invention. In an alternative embodiment, a natural myogenic promoter is utilized, and a skilled artisan is aware how to obtain such promoter sequences from databases including the National Center for Biotechnology Information (NCBI) GenBank database.

The term promoter will be used herein to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

The particular promoter that is employed to control the expression of a Rho kinase polynucleotide, respectively, is not believed to be critical, so long as it is capable of expressing the polynucleotide in the targeted cell at sufficient levels. Thus, where a human cell is targeted, it is preferable to position the polynucleotide-coding region adjacent to and under the control of a promoter that is capable of being expressed in a human cell. Generally speaking, such a promoter might include either a human or viral promoter. However, in specific embodiments, the promoter is operable in fibroblasts, stem cells, smooth muscle cells, cardiomyocytes and/or a combination thereof.

In various embodiments, the human cytomegalovirus (CMV) immediate early gene promoter, the SV40 early promoter and the Rous sarcoma virus long terminal repeat can be used to obtain high-level expression of Rho kinase polynucleotide(s). The use of other viral or mammalian cellular or bacterial phage promoters that are well-known in the art to achieve expression of polynucleotides is contemplated as well, provided that the levels of expression are sufficient to produce a growth inhibitory effect.

By employing a promoter with well-known properties, the level and pattern of expression of a polynucleotide following transfection can be optimized. For example, selection of a promoter that is active in muscle cells permits tissue-specific expression of Rho kinase polynucleotides, respectively. Table 2 lists several elements/promoters that may be employed, in the context of the present invention, to regulate the expression of Rho kinase constructs. This list is not intended to be exhaustive of all the possible elements involved in the promotion of Rho kinase expression but, merely, to be exemplary thereof.

Enhancers were originally detected as genetic elements that increased transcription from a promoter located at a distant position on the same molecule of DNA. This ability to act over a large distance had little precedent in classic studies of prokaryotic transcriptional regulation. Subsequent work showed that regions of DNA with enhancer activity are organized much like promoters. That is, they are composed of many individual elements, each of which binds to one or more transcriptional proteins.

The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Additionally, any promoter/enhancer combination (as per the Eukaryotic Promoter Data Base EPDB) could also be used to drive expression of a SRF construct. Use of a T3, T7 or SP6 cytoplasmic expression system is another possible embodiment. Eukaryotic cells can support cytoplasmic transcription from certain bacteriophage promoters if the appropriate bacteriophage polymerase is provided, either as part of the delivery complex or as an additional genetic expression vector.

TABLE 2
ENHANCER

Immunoglobulin Heavy Chain
Immunoglobulin Light Chain
T-Cell Receptor
HLA DQ a and DQ β
β-Interferon
Interleukin-2
Interleukin-2 Receptor
MHC Class II 5
MHC Class II HLA-DRa
β-Actin
Muscle Creatine Kinase
Prealbumin (Transthyretin)
Elastase I
Metallothionein
Collagenase
Albumin Gene
α-Fetoprotein
τ-Globin
β-Globin
c-fos
c-HA-ras
Insulin
Neural Cell Adhesion Molecule (NCAM)
al-Antitrypsin
H2B (TH2B) Histone
Mouse or Type I Collagen
Glucose-Regulated Proteins (GRP94 and GRP78)
Rat Growth Hormone
Human Serum Amyloid A (SAA)
Troponin I (TN I)
Platelet-Derived Growth Factor
Duchenne Muscular Dystrophy
SV40
Polyoma
Retroviruses
Papilloma Virus
Hepatitis B Virus
Human Immunodeficiency Virus
Cytomegalovirus
Gibbon Ape Leukemia Virus

Further, selection of a promoter that is regulated in response to specific physiologic signals can permit inducible expression of Rho kinase constructs, respectively. For example, with the polynucleotide under the control of the human PAI-1 promoter, expression is inducible by tumor necrosis factor. Table 3 illustrates several promoter/inducer combinations:

TABLE 3

| Element | Inducer |
|---|---|
| MT II Phorbol Ester (TFA) | Heavy metals |
| MMTV (mouse mammary tumor virus) | Glucocorticoids |
| β-Interferon | Poly(rI)XPoly(rc) |
| Adenovirus 5 E2 | Elα |
| c-jun | Phorbol Ester (TPA), H2O2 |
| Collagenase | Phorbol Ester (TPA) |
| Stromelysin | Phorbol Ester (TPA), IL-1 |
| SV40 | Phorbol Ester (TPA) |
| Murine MX Gene | Interferon, Newcastle Disease |
| GRP78 Gene | A23187 |
| α-2-Macroglobulin | IL-6 |
| Vimentin | Serum |
| MHC Class I Gene H-2kB | Interferon |
| HSP70 Ela, SV40 Large T Antigen | Phorbol Ester-TPA |
| Proliferin | |
| Tumor Necrosis Factor | FMA |
| Thyroid Stimulating Hormone a Gene | Thyroid Hormone |

In certain embodiments of the invention, the delivery of an expression vector in a cell may be identified in vitro or in vivo by including a marker in the expression vector. The marker would result in an identifiable change to the transfected cell permitting easy identification of expression. Usually the inclusion of a drug selection marker aids in cloning and in the selection of transformants. Alternatively, enzymes such as herpes simplex virus thymidine kinase (tk) (eukaryotic) or chloramphenicol acetyltransferase (CAT) (prokaryotic) may be employed. Immunologic markers also can be employed. The selectable marker employed is not believed to be important, so long as it is capable of being expressed along with the polynucleotide encoding Rho kinase. Further examples of selectable markers are well known to one of skill in the art.

One typically will include a polyadenylation signal to effect proper polyadenylation of the transcript. The nature of the polyadenylation signal is not believed to be crucial to the successful practice of the invention, and any such sequence may be employed. The inventor has employed the SV40 polyadenylation signal in that it was convenient and known to function well in the target cells employed. Also contemplated as an element of the expression construct is a terminator. These elements can serve to enhance message levels and to minimize read through from the construct into other sequences.

The expression construct may comprise a virus or engineered construct derived from a viral genome. The ability of certain viruses to enter cells via receptor-mediated endocytosis and, in some cases, integrate into the host cell chromosomes, have made them attractive candidates for gene transfer in to mammalian cells. However, because it has been demonstrated that direct uptake of naked DNA, as well as receptor-mediated uptake of DNA complexes, expression vectors need not be viral but, instead, may be any plasmid, cosmid or phage construct that is capable of supporting expression of encoded genes in mammalian cells, such as pUC or Bluescrip™ plasmid series.

C. Rational Drug Design

The goal of rational drug design is to produce structural analogs of biologically active polypeptides or compounds with which they interact (agonists, antagonists, inhibitors, binding partners, etc.). By creating such analogs, it is possible to fashion drugs which are more active or stable than the natural molecules, which have different susceptibility to alteration, or which may affect the function of various other molecules. In one approach, one would generate a three-dimensional structure for Rho kinase (such as ROCK1 selectively), for an uncleavable Rho kinase (such as ROCK1 selectively), for a caspase inhibitor (for ROCK1 selectively), or a fragment thereof. This could be accomplished by x-ray crystallography, computer modeling or by a combination of both approaches. An alternative approach, “alanine scan,” involves the random replacement of residues throughout molecule with alanine, and the resulting affect on function determined.

It also is possible to isolate, for example, a Rho kinase (such as ROCK1 selectively) specific antibody, selected by a functional assay, and then solve its crystal structure. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

Thus, one may design drugs that have improved Rho kinase, such as ROCK1-selective, activity or that act as stimulators, inhibitors, agonists, antagonists or Rho kinase or molecules affected by Rho kinase (such as ROCK1-selective) function. By use of cloned Rho kinase (such as ROCK1 selective) sequences, sufficient amounts of Rho kinase (such as ROCK1 selective) can be produced to perform crystallographic studies. In addition, knowledge of the polypeptide sequences permits computer-employed predictions of structure-function relationships.

The present invention also contemplates the use of Rho kinase and active fragments, and nucleic acids coding therefor, in the screening of compounds for activity in either stimulating Rho kinase activity, overcoming the lack of Rho kinase or blocking the effect of a mutant Rho kinase molecule.

The present invention also encompasses the use of various animal models. By developing or isolating mutant cells lines that fail to express normal Rho kinase, one can, in some embodiments, generate cardiac disease models in mice that will be highly predictive of same in humans and other mammals. Transgenic animals that lack a wild-type Rho kinase may be utilized as models for cardiac disease development and treatment.

Treatment of animals with test compounds will involve the administration of the compound, in an appropriate form, to the animal. Administration will be by any route the could be utilized for clinical or non-clinical purposes, including but not limited to oral, nasal, buccal, rectal, vaginal or topical. Alternatively, administration may be by intratracheal instillation, bronchial instillation, intradermal, subcutaneous, intramuscular, intraperitoneal or intravenous injection. Specifically contemplated are systemic intravenous injection, regional administration via blood or lymph supply and intratumoral injection.

Determining the effectiveness of a compound in vivo may involve a variety of different criteria. Such criteria include, but are not limited to, survival, reduction of tumor burden or mass, arrest or slowing of tumor progression, elimination of tumors, inhibition or prevention of metastasis, increased activity level, improvement in immune effector function and improved food intake.

VIII. Pharmaceutical Compositions and Routes of Administration

Compositions of the present invention may have an effective amount of a specific ROCK1 inhibitor for therapeutic administration for cardiac disease and, in some embodiments, in combination with an effective amount of a compound (second agent) that is an anti-cardiac disease agent. Such compositions will generally be dissolved or dispersed in a pharmaceutically acceptable carrier or aqueous medium.

The phrases “pharmaceutically or pharmacologically acceptable” refer to molecular entities and compositions that do not produce an adverse, allergic or other untoward reaction when administered to an animal, or human, as appropriate. As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredients, its use in the therapeutic compositions is contemplated. Supplementary active ingredients, such as other anti-cancer agents, can also be incorporated into the compositions.

In addition to the compounds formulated for parenteral administration, such as intravenous or intramuscular injection, other pharmaceutically acceptable forms include, e.g., tablets or other solids for oral administration; time release capsules; and any other form currently used, including cremes, lotions, mouthwashes, inhalants and the like.

The expression vectors and delivery vehicles of the present invention may include classic pharmaceutical preparations. Administration of these compositions according to the present invention will be via any common route so long as the target tissue is available via that route. This includes oral, nasal, buccal, rectal, vaginal or topical. Alternatively, administration may be by orthotopic, intradermal, subcutaneous, intramuscular, intraperitoneal or intravenous injection. Such compositions would normally be administered as pharmaceutically acceptable compositions, described supra.

The vectors of the present invention are advantageously administered in the form of injectable compositions either as liquid solutions or suspensions; solid forms suitable for solution in, or suspension in, liquid prior to injection also may be prepared. These preparations also may be emulsified. A typical composition for such purposes comprises a 50 mg or up to about 100 mg of human serum albumin per milliliter of phosphate buffered saline. Other pharmaceutically acceptable carriers include aqueous solutions, non-toxic excipients, including salts, preservatives, buffers and the like. Examples of non-aqueous solvents are propylene glycol, polyethylene glycol, vegetable oil and injectable organic esters, such as theyloleate. Aqueous carriers include water, alcoholic/aqueous solutions, saline solutions, parenteral vehicles such as sodium chloride, Ringer's dextrose, etc. Intravenous vehicles include fluid and nutrient replenishers. Preservatives include antimicrobial agents, anti-oxidants, chelating agents and inert gases. The pH and exact concentration of the various components in the pharmaceutical are adjusted according to well-known parameters.

Additional formulations are suitable for oral administration. Oral formulations include such typical excipients as, for example, pharmaceutical grades of mannitol, lactose, starch, magnesium stearate, sodium saccharine, cellulose, magnesium carbonate and the like. The compositions take the form of solutions, suspensions, tablets, pills, capsules, sustained release formulations or powders. When the route is topical, the form may be a cream, ointment, salve or spray.

An effective amount of the therapeutic agent is determined based on the intended goal. The term “unit dose” refers to a physically discrete unit suitable for use in a subject, each unit containing a predetermined quantity of the therapeutic composition calculated to produce the desired response in association with its administration, i.e., the appropriate route and treatment regimen. The quantity to be administered, both according to number of treatments and unit dose, depends on the subject to be treated, the state of the subject and the protection desired. Precise amounts of the therapeutic composition also depend on the judgment of the practitioner and are peculiar to each individual.

All of the essential materials and reagents required for delivery of a specific ROCK1 inhibitor may be assembled together in a kit. When the components of the kit are provided in one or more liquid solutions, the liquid solution preferably is an aqueous solution, with a sterile aqueous solution being particularly preferred.

For in vivo use, an anti-cardiac disease agent may be formulated into a single or separate pharmaceutically acceptable syringeable composition. In this case, the container means may itself be an inhalant, syringe, pipette, eye dropper, or other such like apparatus, from which the formulation may be applied to an infected area of the body, such as the lungs, injected into an animal, or even applied to and mixed with the other components of the kit.

The components of the kit may also be provided in dried or lyophilized forms. When reagents or components are provided as a dried form, reconstitution generally is by the addition of a suitable solvent. It is envisioned that the solvent also may be provided in another container means. The kits of the invention may also include an instruction sheet defining administration of the gene therapy and/or the anti-cardiac disease drug.

The kits of the present invention also will typically include a means for containing the vials in close confinement for commercial sale such as, e.g., injection or blow-molded plastic containers into which the desired vials are retained. Irrespective of the number or type of containers, the kits of the invention also may comprise, or be packaged with, an instrument for assisting with the injection/administration or placement of the ultimate complex composition within the body of an animal. Such an instrument may be an inhalant, syringe, pipette, forceps, measured spoon, eye-dropper or any such medically approved delivery vehicle.

The active compounds of the present invention will often be formulated for parenteral administration, e.g., formulated for injection via the intravenous, intramuscular, subcutaneous, or even intraperitoneal routes. The preparation of an aqueous composition that contains a second agent(s) as active ingredients will be known to those of skill in the art in light of the present disclosure. Typically, such compositions can be prepared as injectables, either as liquid solutions or suspensions; solid forms suitable for using to prepare solutions or suspensions upon the addition of a liquid prior to injection can also be prepared; and the preparations can also be emulsified.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The active compounds may be formulated into a composition in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

In certain cases, the therapeutic formulations of the invention could also be prepared in forms suitable for topical administration, such as in cremes and lotions.

Upon formulation, solutions will be administered in a manner compatible with the dosage formulation and in such amount as is therapeutically effective. The formulations are easily administered in a variety of dosage forms, such as the type of injectable solutions described above, with even drug release capsules and the like being employable.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media that can be employed will be known to those of skill in the art in light of the present disclosure. For example, one dosage could be dissolved in 1 mL of isotonic NaCl solution and either added to 1000 mL of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

Targeting of cardiovascular tissues may be accomplished in any one of a variety of ways. Plasmid vectors and retroviral vectors, adenovirus vectors, and other viral vectors all present means by which to target cardiovascular tissue. The inventors anticipate particular success for the use of liposomes to target Rho kinase, caspase inhibitor and/or uncleavable Rho kinase to cells, to cardiac tissue. For example, DNA encoding Rho kinase or uncleavable Rho kinase may be complexed with liposomes in the manner described above, and this DNA/liposome complex is injected into patients with cardiac disease, intravenous injection can be used to direct the gene to all cell. Directly injecting the liposome complex into the proximity of the diseased tissue can also provide for targeting of the complex with some forms of cardiac disease. Of course, the potential for liposomes that are selectively taken up by a population of cells exists, and such liposomes will also be useful for targeting the gene.

Those of skill in the art will recognize that the best treatment regimens for using Rho kinase (ROCK1), an agent that inhibits ROCK1, an uncleavable Rho kinase, and/or a caspase inhibitor to prevent and/or to treat diseased cardiac tissue can be straightforwardly determined. This is not a question of experimentation, but rather one of optimization, which is routinely conducted in the medical arts. In one exemplary embodiment, in vivo studies in nude mice provide a starting point from which to begin to optimize the dosage and delivery regimes. The frequency of injection will initially be once a week, as was done some mice studies. However, this frequency might be optimally adjusted from one day to every two weeks to monthly, depending upon the results obtained from the initial clinical trials and the needs of a particular patient. Human dosage amounts can initially be determined by extrapolating from the amount of Rho kinase used in mice. In certain embodiments it is envisioned that the dosage may vary from between about 1 mg Rho kinase DNA/Kg body weight to about 5000 mg Rho kinase DNA/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, 5000 mg/Kg body weight. In other embodiments, it is envisaged that higher does may be used, such doses may be in the range of about 5 mg Rho kinase DNA/Kg body to about 20 mg Rho kinase DNA/Kg body. In other embodiments the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

IX. Screening For Modulators Of the Protein Function

The present invention further comprises methods for identifying modulators of the function of Rho kinase, such as the exemplary Rho kinase ROCK1. In a particular embodiment of the invention, the inhibitor does not inhibit ROCK2. These assays may comprise random screening of large libraries of candidate substances; alternatively, the assays may be used to focus on particular classes of compounds selected with an eye towards structural attributes that are believed to make them more likely to modulate the function of Rho kinase.

By function, it is meant that one may assay, for example, the ability to specifically inhibit expression of ROCK1, specifically inhibit activity of ROCK1, specifically inhibit cleavage of ROCK1, or a combination thereof. One may also assay for more global effects, such as amelioration and/or prevention of at least one cardiac disease symptom.

To identify a Rho kinase modulator, one generally will characterize the function, activity, expression, or cleavage status of Rho kinase in the presence and absence of the candidate substance, a modulator defined as any substance that alters function. For example, a method generally comprises:

providing a candidate modulator;

admixing the candidate modulator with an isolated compound or cell, or a suitable experimental animal;

measuring one or more characteristics of the compound, cell or animal in step (c); and

comparing the characteristic measured in step (c) with the characteristic of the compound, cell or animal in the absence of said candidate modulator,

wherein a difference between the measured characteristics indicates that said candidate modulator is, indeed, a modulator of the compound, cell or animal.

Assays may be conducted in cell free systems, in isolated cells, or in organisms including transgenic animals.

It will, of course, be understood that all the screening methods of the present invention are useful in themselves notwithstanding the fact that effective candidates may not be found. The invention provides methods for screening for such candidates, not solely methods of finding them.

1. Modulators

As used herein the term “candidate substance” refers to any molecule that may potentially inhibit Rho kinase expression, function, activity, or cleavage status. The candidate substance may be a protein or fragment thereof, a small molecule, or even a nucleic acid molecule. It may prove to be the case that the most useful pharmacological compounds will be compounds that are structurally related to inhibitors of other Rho kinases or any kinase. Using lead compounds to help develop improved compounds is know as “rational drug design” and includes not only comparisons with know inhibitors and activators, but predictions relating to the structure of target molecules.

The goal of rational drug design is to produce structural analogs of biologically active polypeptides or target compounds. By creating such analogs, it is possible to fashion drugs, which are more active or stable than the natural molecules, which have different susceptibility to alteration or which may affect the function of various other molecules. In one approach, one would generate a three-dimensional structure for a target molecule, or a fragment thereof. This could be accomplished by x-ray crystallography, computer modeling or by a combination of both approaches.

It also is possible to use antibodies to ascertain the structure of a target compound activator or inhibitor. In principle, this approach yields a pharmacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable modulators include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the modulating compounds initially identified, the inventors also contemplate that other sterically similar compounds may be formulated to mimic the key portions of the structure of the modulators. Such compounds, which may include peptidomimetics of peptide modulators, may be used in the same manner as the initial modulators.

An inhibitor according to the present invention may be one that exerts its inhibitory or activating effect upstream, downstream or directly on Rho kinase. Regardless of the type of inhibitor or activator identified by the present screening methods, the effect of the inhibition or activator by such a compound results in inhibition of Rho kinase expression, activity, function, cleavage status, or amelioration and/or prevention of at least one cardiac disease symptom as compared to that observed in the absence of the added candidate substance.

2. In vitro Assays

A quick, inexpensive and easy assay to run is an in vitro assay. Such assays generally use isolated molecules, can be run quickly and in large numbers, thereby increasing the amount of information obtainable in a short period of time. A variety of vessels may be used to run the assays, including test tubes, plates, dishes and other surfaces such as dipsticks or beads.

One example of a cell free assay is a binding assay. While not directly addressing function, the ability of a modulator to bind to a target molecule in a specific fashion is strong evidence of a related biological effect. For example, binding of a molecule to a target may, in and of itself, be inhibitory, due to steric, allosteric or charge-charge interactions. The target may be either free in solution, fixed to a support, expressed in or on the surface of a cell. Either the target or the compound may be labeled, thereby permitting determining of binding. Usually, the target will be the labeled species, decreasing the chance that the labeling will interfere with or enhance binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

3. In cyto Assays

The present invention also contemplates the screening of compounds for their ability to modulate Rho kinase in cells. Various cell lines can be utilized for such screening assays, including cells specifically engineered for this purpose. In a particular exemplary embodiment, cardiac cells are utilized and, in further specific exemplary embodiments, Rho kinase is cleaved in the cell. In specific embodiments, apoptosis of the cell is assayed.

Depending on the assay, culture may be required. The cell is examined using any of a number of different physiologic assays. Alternatively, molecular analysis may be performed, for example, looking at protein expression, mRNA expression (including differential display of whole cell or polyA RNA) and others.

4. In vivo Assays

In vivo assays involve the use of various animal models, including transgenic animals that have been engineered to have specific defects, or carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for modulators may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies a modulator. The characteristics may be any of those discussed above with regard to the function of a particular compound (e.g., enzyme, receptor, hormone) or cell (e.g., growth, tumorigenicity, survival), or instead a broader indication such as behavior, anemia, immune response, etc.

The present invention provides methods of screening for a candidate substance that inhibits Rho kinase expression, activity, function, or cleavage or that ameliorates and/or prevents at least one symptom of cardiac disease. In particular embodiments, the present invention is directed to a method for determining the ability of a candidate substance to inhibit Rho kinase cleavage, generally including the steps of: administering a candidate substance to the animal; and determining the ability of the candidate substance to reduce one or more characteristics of cardiac disease.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve a variety of different criteria. Also, measuring toxicity and dose response can be performed in animals in a more meaningful fashion than in in vitro or in cyto assays.

Transgenic non-human animals (e.g., mammals) of the invention can be of a variety of species including murine (rodents (e.g., mice, rats), avian (chicken, turkey, fowl), bovine (beef, cow, cattle), ovine (lamb, sheep, goats), porcine (pig, swine), and piscine (fish). In a preferred embodiment, the transgenic animal is a rodent, such as a mouse or a rat.

X. Transgenic Animals

Detailed methods for generating non-human transgenic animal are described herein and in the section entitled “Examples” below. Transgenic gene constructs can be introduced into the germ line of an animal to make a transgenic mammal. For example, one or several copies of the construct may be incorporated into the genome of a mammalian embryo by standard transgenic techniques.

Any non-human animal can be used in the methods described herein. Preferred mammals are rodents, e.g., rats or mice.

In an exemplary embodiment, the “transgenic non-human animals” of the invention are produced by introducing transgenes into the germline of the non-human animal. Embryonal target cells at various developmental stages can be used to introduce transgenes. Different methods are used depending on the stage of development of the embryonal target cell. The specific line(s) of any animal used to practice this invention are selected for general good health, good embryo yields, good pronuclear visibility in the embryo, and good reproductive fitness. In addition, the haplotype is a significant factor.

Introduction of the transgene into the embryo can be accomplished by any means known in the art such as, for example, microinjection, electroporation, or lipofection. For example, the Fc receptor transgene can be introduced into a mammal by microinjection of the construct into the pronuclei of the fertilized mammalian egg(s) to cause one or more copies of the construct to be retained in the cells of the developing mammal(s). Following introduction of the transgene construct into the fertilized egg, the egg may be incubated in vitro for varying amounts of time, or reimplanted into the surrogate host, or both. In vitro incubation to maturity is within the scope of this invention. One common method in to incubate the embryos in vitro for about 1-7 days, depending on the species, and then reimplant them into the surrogate host.

The progeny of the transgenically manipulated embryos can be tested for the presence of the construct by Southern blot analysis of the segment of tissue. If one or more copies of the exogenous cloned construct remains stably integrated into the genome of such transgenic embryos, it is possible to establish permanent transgenic mammal lines carrying the transgenically added construct.

The litters of transgenically altered mammals can be assayed after birth for the incorporation of the construct into the genome of the offspring. Preferably, this assay is accomplished by hybridizing a probe corresponding to the DNA sequence coding for the desired recombinant protein product or a segment thereof onto chromosomal material from the progeny. Those mammalian progeny found to contain at least one copy of the construct in their genome are grown to maturity.

For the purposes of this invention a zygote is essentially the formation of a diploid cell which is capable of developing into a complete organism. Generally, the zygote will be comprised of an egg containing a nucleus formed, either naturally or artificially, by the fusion of two haploid nuclei from a gamete or gametes. Thus, the gamete nuclei must be ones which are naturally compatible, i.e., ones which result in a viable zygote capable of undergoing differentiation and developing into a functioning organism. Generally, a euploid zygote is preferred. If an aneuploid zygote is obtained, then the number of chromosomes should not vary by more than one with respect to the euploid number of the organism from which either gamete originated.

In addition to similar biological considerations, physical ones also govern the amount (e.g., volume) of exogenous genetic material which can be added to the nucleus of the zygote or to the genetic material which forms a part of the zygote nucleus. If no genetic material is removed, then the amount of exogenous genetic material which can be added is limited by the amount which will be absorbed without being physically disruptive. Generally, the volume of exogenous genetic material inserted will not exceed about 10 picoliters. The physical effects of addition must not be so great as to physically destroy the viability of the zygote. The biological limit of the number and variety of DNA sequences will vary depending upon the particular zygote and functions of the exogenous genetic material and will be readily apparent to one skilled in the art, because the genetic material, including the exogenous genetic material, of the resulting zygote must be biologically capable of initiating and maintaining the differentiation and development of the zygote into a functional organism.

The number of copies of the transgene constructs which are added to the zygote is dependent upon the total amount of exogenous genetic material added and will be the amount which enables the genetic transformation to occur. Theoretically only one copy is required; however, generally, numerous copies are utilized, for example, 1,000-20,000 copies of the transgene construct, in order to insure that one copy is functional. As regards the present invention, there will often be an advantage to having more than one functioning copy of each of the inserted exogenous DNA sequences to enhance the phenotypic expression of the exogenous DNA sequences.

Any technique which allows for the addition of the exogenous genetic material into nucleic genetic material can be utilized so long as it is not destructive to the cell, nuclear membrane or other existing cellular or genetic structures. The exogenous genetic material is preferentially inserted into the nucleic genetic material by microinjection. Microinjection of cells and cellular structures is known and is used in the art.

Reimplantation is accomplished using standard methods. Usually, the surrogate host is anesthetized, and the embryos are inserted into the oviduct. The number of embryos implanted into a particular host will vary by species, but will usually be comparable to the number of off spring the species naturally produces.

Transgenic offspring of the surrogate host may be screened for the presence and/or expression of the transgene by any suitable method. Screening is often accomplished by Southern blot or Northern blot analysis, using a probe that is complementary to at least a portion of the transgene. Western blot analysis using an antibody against the protein encoded by the transgene may be employed as an alternative or additional method for screening for the presence of the transgene product. Typically, DNA is prepared from tail tissue and analyzed by Southern analysis or PCR for the transgene. Alternatively, the tissues or cells believed to express the transgene at the highest levels are tested for the presence and expression of the transgene using Southern analysis or PCR, although any tissues or cell types may be used for this analysis.

Alternative or additional methods for evaluating the presence of the transgene include, without limitation, suitable biochemical assays such as enzyme and/or immunological assays, histological stains for particular marker or enzyme activities, flow cytometric analysis, and the like. Analysis of the blood may also be useful to detect the presence of the transgene product in the blood, as well as to evaluate the effect of the transgene on the levels of various types of blood cells and other blood constituents.

Progeny of the transgenic animals may be obtained by mating the transgenic animal with a suitable partner, or by in vitro fertilization of eggs and/or sperm obtained from the transgenic animal. Where mating with a partner is to be performed, the partner may or may not be transgenic and/or a knockout; where it is transgenic, it may contain the same or a different transgene, or both. Alternatively, the partner may be a parental line. Where in vitro fertilization is used, the fertilized embryo may be implanted into a surrogate host or incubated in vitro, or both. Using either method, the progeny may be evaluated for the presence of the transgene using methods described above, or other appropriate methods.

The transgenic animals produced in accordance with the present invention will include exogenous genetic material. As set out above, the exogenous genetic material will, in certain embodiments, be a DNA sequence which results in the production of an Fc receptor. Further, in such embodiments the sequence will be attached to a transcriptional control element, e.g., a promoter, which preferably allows the expression of the transgene product in a specific type of cell.

Retroviral infection can also be used to introduce transgene into a non-human animal. The developing non-human embryo can be cultured in vitro to the blastocyst stage. During this time, the blastomeres can be targets for retroviral infection (Jaenich, R. (1976) PNAS 73:1260-1264). Efficient infection of the blastomeres is obtained by enzymatic treatment to remove the zona pellucida (Manipulating the Mouse Embryo, Hogan eds. (Cold Spring Harbor Laboratory Press, Cold Spring Harbor, 1986). The viral vector system used to introduce the transgene is typically a replication-defective retrovirus carrying the transgene (Jahner et al. (1985) PNAS 82:6927-6931; Van der Putten et al. (1985) PNAS 82:6148-6152). Transfection is easily and efficiently obtained by culturing the blastomeres on a monolayer of virus-producing cells (Van der Putten, supra; Stewart et al. (1987) EMBO J. 6:383-388). Alternatively, infection can be performed at a later stage. Virus or virus-producing cells can be injected into the blastocoele (Jahner et al. (1982) Nature 298:623-628). Most of the founders will be mosaic for the transgene since incorporation occurs only in a subset of the cells which formed the transgenic non-human animal. Further, the founder may contain various retroviral insertions of the transgene at different positions in the genome which generally will segregate in the offspring. In addition, it is also possible to introduce transgenes into the germ line by intrauterine retroviral infection of the midgestation embryo (Jahner et al. (1982) supra).

A third type of target cell for transgene introduction is the embryonal stem cell (ES). ES cells are obtained from pre-implantation embryos cultured in vitro and fused with embryos (Evans et al. (1981) Nature 292:154-156; Bradley et al. (1984) Nature 309:255-258; Gossler et al. (1986) PNAS 83: 9065-9069; and Robertson et al. (1986) Nature 322:445-448). Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Role of Rho Kinase in Mediating Hypertrophic Responses Under Pressure Overload

Rho kinase inhibitor, Y27632, inhibits protein kinases A and C, but with about 200 times lower affinity than that for Rho kinase (Uehata et al., 1997; Ishizaki et al., 2000). Using this compound and fasudil, another chemical Rho kinase inhibitor, several studies suggest that Rho kinase inhibitors may have therapeutic benefits for the treatment of hypertension, vascular proliferative disorders and cancer (Kuwahara et al., 1999; Uehata et al., 1997; Itoh et al., 1999; Sawada et al., 2000). To investigate the role of Rho kinase in mediating hypertrophic responses under pressure overload, the present inventors employed a genetic approach, which provides more direct and conclusive evidence than Y27632 treatment and allows determination of potential differential effects of Rho kinase isoforms.

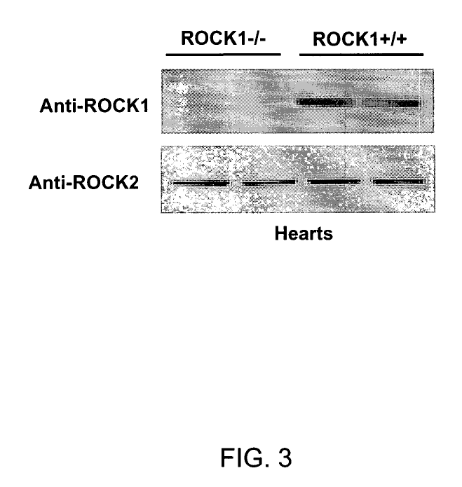

FIG. 3 demonstrates expression analysis of ROCK1 and ROCK2 in ROCK1 homozygous knockout adult hearts. Two wild-type littermates (ROCK1+/+) were used as controls. Equal amounts of proteins from heart homogenates were analyzed by Western blotting using anti-ROCK1 or anti-ROCK2 directed against the coiled-coil region of ROCK1 or ROCK2 respectively. ROCK1 expression was undetectable in homozygous knockout hearts (ROCK1−/−) while the ROCK2 expression level was unchanged.

### Example 2

### Generation of ROCK1 Knockout Mice

ROCK1 was knocked out because of its enriched expression pattern in the mouse developing heart (Wei et al., 2001). ROCK1 knockout mice were successfully generated. The coding sequence of β-galactosidase was inserted in frame downstream of residue 180 followed by PGK-Neo. As the entire kinase domain is contained within residues 76-338, the majority of the kinase domain and the following coiled-coil and the PH domains are knocked out. Two independent ES clones have shown transmission through the germ line to establish heterozygous ROCK1+/− mouse strains. Homozygous knockout mice are viable and morphologically indistinguishable from wild-type littermates. This lack of cardiac phenotype in ROCK1 homozygous knockout mice was not totally unexpected, due to the presence of ROCK2 (FIG. 3).

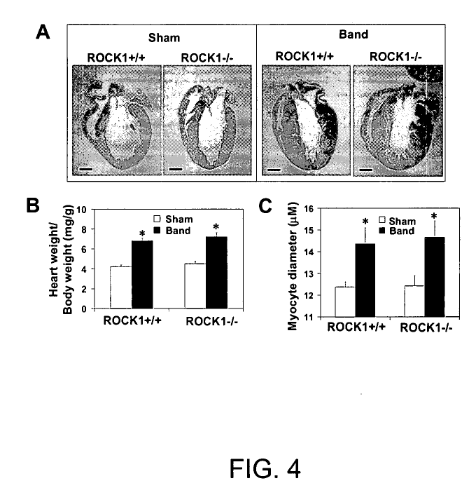

ROCK1-deficient mice develop cardiac hypertrophy in response to pressure overload (FIG. 4). The absence of a developmental cardiac phenotype in ROCK1 knockout mice allows examination of the role of ROCK1 in the pathophysiological settings such as pressure overload by aortic constriction. Both ROCK1−/− and control mice received a comparable load, based on the right-to-left carotid artery flow velocity ratio (more than 4:1) after constricting the transverse aorta. The body weight and the heart weight were not significantly different between control and ROCK1^{-/-} mice before banding. Three weeks after banding, both ROCK1−/− and control mice developed compensated concentric hypertrophy (FIG. 4). Cardiac myocyte diameter was significantly increased in both ROCK1^{-/-} and control mice. These results indicate that cardiac hypertrophy induced by pressure overload occurs in the absence of ROCK1 activation and that ROCK1 is not involved in the regulation of myocyte size in response to pressure overload.

FIG. 4 demonstrates that cardiac hypertrophy develops in response to pressure overload in ROCK1^{-/-} mice. FIG. 4A shows heart sections from ROCK1^{-/-} and control mice after three-week aortic banding. Bar, 1 mm. FIG. 4B shows quantitation of heartibody weight ratios from ROCK1^{-/-} and control mice after three-week aortic banding (n=5 for each group). In FIG. 4C, there are cardiomyocyte diameters from ROCK1^{-/-} and control mice after three-week aortic banding. Myocyte diameter was measured using transnuclear width at the mid-ventricular level (n=200 for each condition).

### Example 3

### ROCK1 Deficient Mice Exhibit Reduced Hypertrophic Marker Induction, Reduced Apoptosis and Improved Cardiac Contractile Functions Compared to Control Mice in Response to Pressure Overload

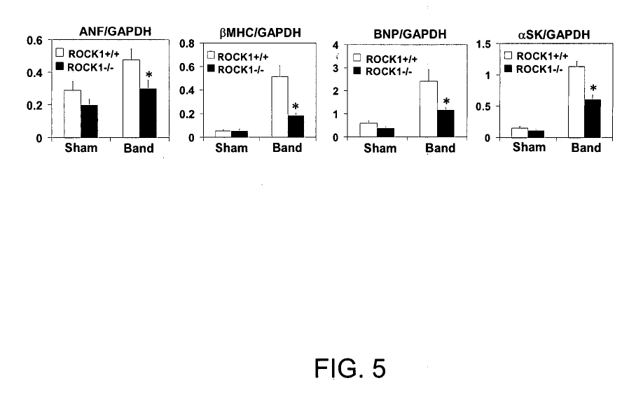

Pathological cardiac hypertrophy is characterized by a prototypical change in gene expression patterns such as ANF, BNP, βMHC and skeletal α-actin. Interestingly, the increases in the expression of these hypertrophic markers in ROCK1^{-/-} mice were significantly lower than in control mice in response to pressure overload (FIG. 5). These results indicate that pressure overload induces cardiac hypertrophy in ROCK1^{-/-} mice and produces reduced pathological changes in the gene expression profile compared to control mice.

FIG. 5 demonstrates real-time RT-PCR analysis of cardiac hypertrophic markers. RNA samples were prepared from ROCK1^{-/-} and control hearts after three-week aortic banding (n=3-4 for each group). Quantitative RT-PCR analysis was performed using the ABI Prism 7700 sequence detection system (Perkin Elmer). The levels of the transcripts were normalized to that of GAPDH.

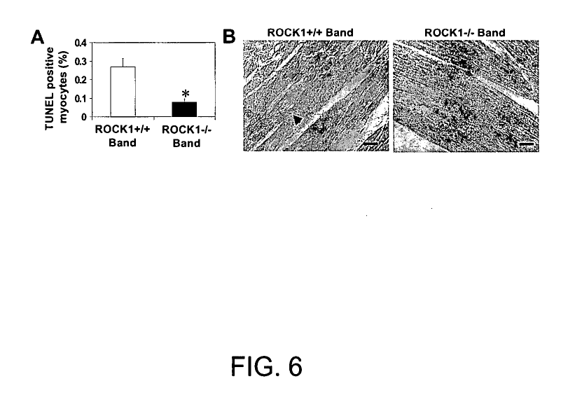

To evaluate the frequency of cell death in the myocardium, TUNEL staining was performed on ROCK1^{-/-} and control hearts under pressure overload (FIG. 6). No significant difference in basal levels of TUNEL-positive cells between control and ROCK1^{-/-} mice was observed. Three weeks after aortic banding, significantly less TUNEL-positive myocytes were observed in the myocardium of ROCK1^{-/-} mice than that from control mice (FIG. 6). The level of interstitial fibrosis was also significantly decreased in ROCK1^{-/-} hearts than in control hearts (FIG. 6). These results suggest that activation of ROCK1 by pressure overload facilitates an apoptotic response and cardiac remodeling.

Cardiac function of ROCK1^{-/-} mice by non-invasive Doppler echocardiography was also assessed (Table 1). The contractile function of the left ventricle (peak aortic outflow velocity and mean acceleration) was not significantly different in ROCK1^{-/-} vs. control mice under sham condition. Interestingly, pressure overload caused a smaller decrease in peak aortic flow velocity in ROCK1^{-/-} mice (22.9%) than in control mice (31.4%). Heart rate was significantly increased in ROCK1^{-/-} mice under pressure overload, which may contribute in part to the difference between ROCK1^{-/-} and control mice in the load-induced fall of peak flow velocity. These results suggest that ROCK1^{-/-} mice tolerate better pressure overload than control mice do.

TABLE 4
Pulsed doppler analysis of flow velocity in ROCK −/− mice

| | ROCK1 +/+ | | | ROCK1 −/− | | |
|---|---|---|---|---|---|---|
| | Sham | Band | % | Sham | Band | % |
| | (n = 5) | (n = 7) | Change | (n = 5) | (n = 7) | Change |
| Heart rate (bpm) | 367 ± 21 | 389 ± 16 | NS | 407 ± 21 | 479 ± 17 | 17.7* |
| E-peak velocity (cm/s) | 87.4 ± 4.2 | 101.9 ± 4.6 | NS | 73.2 ± 0.9 | 91.5 ± 9.9 | NS |
| E/A ratio | 1.79 ± 0.19 | 2.91 ± 0.56 | NS | 1.32 ± 0.04 | 1.65 ± 0.23 | NS |
| Peak aortic flow V (cm/s) | 113 ± 2.3 | 77.5 ± 0.9 | −31.4* | 111 ± 7.1 | 85.5 ± 3.9 | −22.9* |
| Mean acceleration | 8748 ± 866 | 7194 ± 302 | NS | 9173 ± 1550 | 8214 ± 638 | NS |
| (cm/s²⁾ | | | | | | |

Data are presented as mean ± SE.
E-peak: early-peak;
E/A ratio: early/atrial ratio;
V: velocity.
*P < 0.05 banded vs. sham.

Taken together, characterization of ROCK1^{-/-} mice under pressure overload shows the following: activation of ROCK1 is not required for the increase in myocyte size induced by mechanical stress, but activation of ROCK1 potentiates the transition from compensatory to decompensatory stages by stimulating pathological changes in the gene expression profile, myocyte apoptosis and interstitial fibrosis; in specific embodiments, it also depresses myocyte contractile function in response of pressure overload. Additional biochemical, cellular and physiological analyses are performed on ROCK1^{-/-} mice to further characterize the role of ROCK1 in mediating apoptotic responses. The following Examples reveal an exemplary mechanism leading to the activation of ROCK1, which then plays a contributory role to cardiomyocyte apoptosis under pathophysiological conditions.

### Example 4

### ROCK1 is Cleaved in Human Failing Hearts and Ventricular Unloading Attenuated the Cleavage

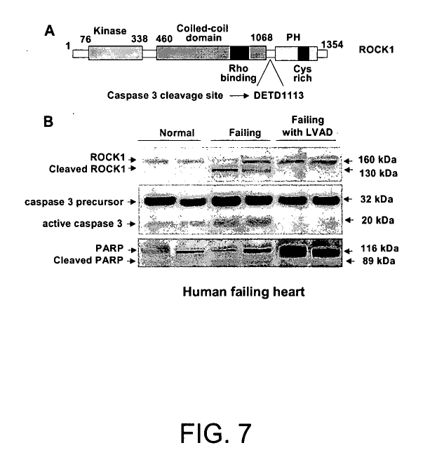

FIG. 7 illustrates that ROCK1 is cleaved in human failing hearts and ventricular unloading attenuates the cleavage. FIG. 7A provides a schematic diagram of ROCK1 cleavage. The consensus recognition sequence for caspase 3 in human ROCK1 (DETD1113) is conserved in mouse and rat, and is not present in ROCK2.

As mentioned previously, three recent studies have shown that caspase 3 activation in apoptotic non-cardiac cells leads to ROCK1 cleavage and RhoA-independent activation (Coleman et al., 2001; Ueda et al., 2001; Sebbagh et al., 2001). To investigate whether this molecular event could also occur in human heart disease, the present inventors examined ventricular myocardial samples from thirteen patients with end-stage heart failure at the time of transplant, seven patients who died of non-cardiac causes (control group) and ten patients with end-stage heart failure who had been maintained on left ventricular assist device (LVAD) support until transplant. FIG. 7B shows representative Western blots of hearts samples. Cleavage of ROCK1, caspase 3, and poly(ADP-ribose) polymerase (PARP) (a well established caspase 3 substrate) was observed in human failing hearts, but not in normal hearts or in failing hearts unloaded by LVAD support. This suggests that caspase 3 activation relates to myocardial mechanical overload. More specifically, Western blot analysis using a polyclonal antibody directed against the coiled-coil region of ROCK1 detected a fragment of 130 kDa in addition to the 160 kDa native protein in all failing hearts without LVAD support. In contrast, normal hearts or failing hearts with LVAD support contained only the 160 kDa native protein. These results show correlation of ROCK1 cleavage with caspase 3 activation in human failing hearts and indicates that ROCK1 is cleaved by caspase 3.

### Example 5

### ROCK1 is Cleaved in Cultured Neonatal Rat Cardiomyocytes Treated with Doxorubicin or Infected with an Adenovirus Encoding a Conditional Caspase 3

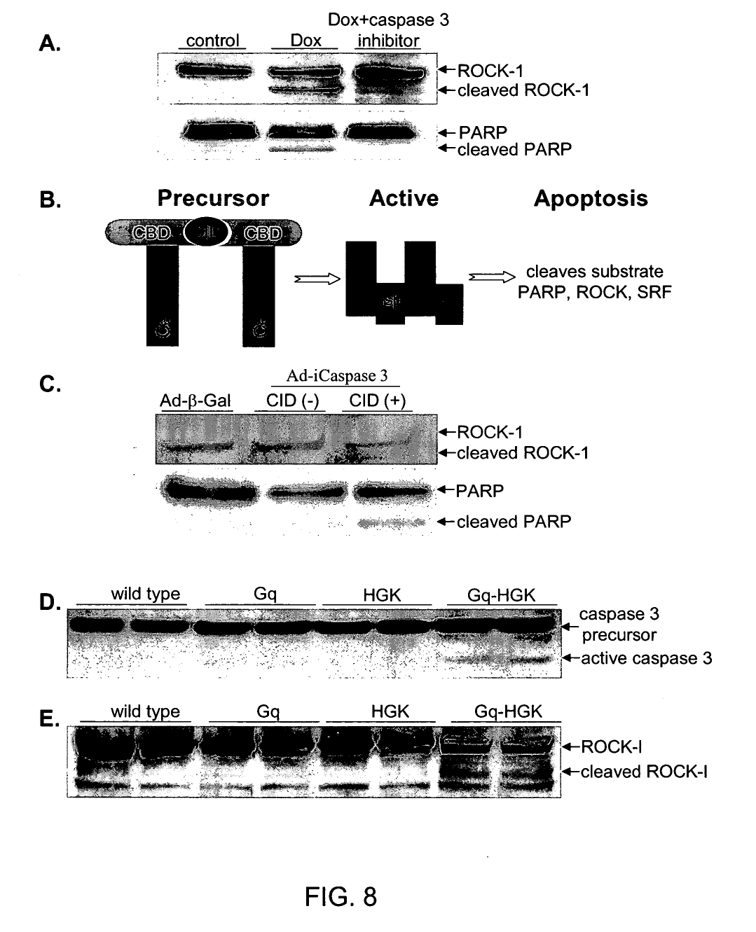

FIG. 8 shows that ROCK1 is cleaved in cultured neonatal rat cardiomyocytes treated with doxorubicin or infected with an adenovirus encoding a conditional caspase 3. To determine if ROCK1 is cleaved by caspase 3 in apoptotic cardiomyocytes, the present inventors subjected cultured rat neonatal cardiomyocytes to doxorubicin treatment, which is a potent apoptotic inducer. The 130 kDa cleavage fragment of ROCK1 was detected in doxorubicin-treated myocytes as well as the cleaved PARP. In contrast, cleavage of ROCK2 was not observed. In addition, cleavage of ROCK1 is caspase 3-dependent, as the 130 kDa species was not detected in the presence of caspase 3 inhibitor, Z-VAD.

To analyze direct effect of caspase 3 activation on ROCK1 cleavage, the present inventors introduced a conditional caspase 3 via an adenoviral-mediated expression system in cultured rat neonatal cardiomyocytes. The adenoviral vector, Ad-G/iCasp3, has been described previously (Shariat et al., 2001; Mallet et al., 2002). This vector expresses a conditional caspase 3 (iCasp3), in which the human caspase 3 was fused to a modified FK506-binding domain, containing a binding site for a non-toxic chemical inducer of dimerization (CID), AP20187. Addition of CID leads to the aggregation and activation this caspase, and a rapid apoptosis in prostate cancer cells (Shariat et al., 2001; Mallet et al., 2002). This is depicted in FIG. 8C. The 130 kDa cleavage fragment of ROCK1 was detected in cardiomyocytes infected with Ad-G/iCasp3 and treated with CID, indicating that activation of caspase 3 is sufficient to cleave ROCK1 in cultured cardiomyocytes.

To assess ROCK-1 cleavage in vivo, the present inventors took advantage of three transgenic mouse lines with different degrees of heart failure as a correlate shared between ROCK-1 cleavage and the severity of heart failure. Hearts from αMHC-Gq, αMHC-HGK, and bi-transgenic αMHC-Gq-HGK were analyzed using Western blot. As indicated in FIGS. 8D and 8E, a significant increase in caspase 3 activity was observed in hearts from bi-transgenic mice, which coincided with the appearance of the clipped 130 kDa ROCK-1 fragment. Cleavage fragments were not detected in HGK or Gq mice, although there was a slight increase in caspase 3 activity in Gq mice hearts. These data were consistent with a previous report, which demonstrated that the combination of cardiac Gq with HGK overexpression led to ventricular dilatation and early lethality with severe apoptosis (Xie et al., 2004). In contrast, apoptosis was observed at a low level in hearts with overexpression of Gq and was virtually undetectable in cardiac transgenic HGK mice (Xie et al., 2004). Therefore, ROCK-1 cleavage in vivo coincided in mouse genetic models with increased levels of caspase 3 activity.

### Example 6

### Expression of a ROCK1 Mutant (ROCKδ1), Which Mimics Caspase 3 Cleaved Fragment, Leads to Caspase 3 Activation in Cultured Neonatal Rat Cardiomyocytes

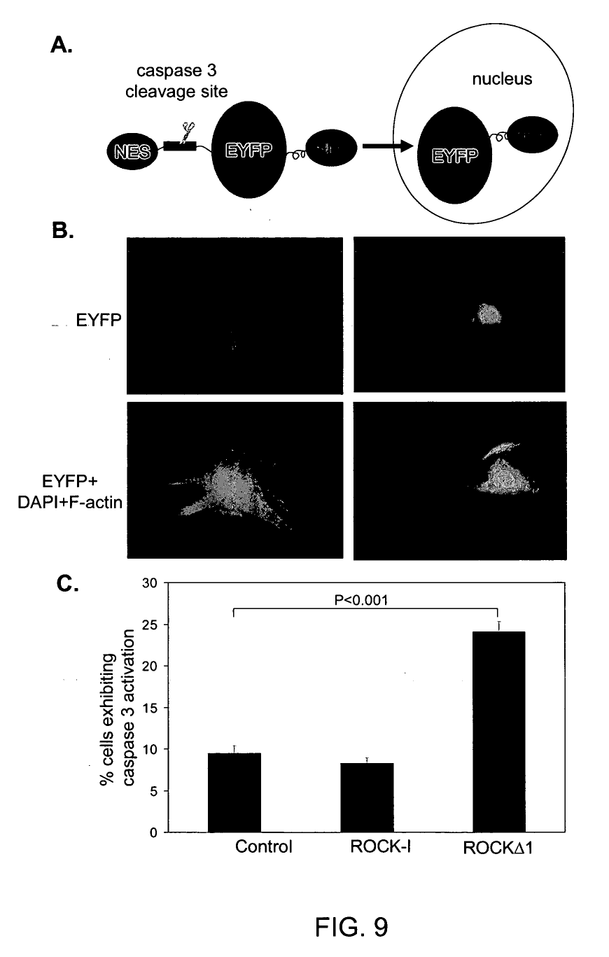

FIG. 9 shows that expression of a ROCKΔ1 mutant, which mimics caspase 3 cleaved fragment, leads to caspase 3 activation in cultured neonatal rat cardiomyocytes. To determine whether ROCK1 activation by caspase 3 cleavage is able to activate the caspase cascade, thereby constituting an amplification loop of apoptotic responses, the present inventors transfected rat neonatal cardiomyocytes with a plasmid encoding a C-terminally truncated mutant (1-1080) of ROCK1 (ROCK1Δ1) that corresponds closely to the fragment generated by caspase 3 cleavage (1-1113), and a second plasmid, pCaspase3-sensor encoding a fluorescent (EYFP) fusion protein (FIG. 9A). ROCK1Δ1 as well as the ROCK1 mutant (1-1113) have been demonstrated as a constitutively active form of ROCK1 (Coleman et al., 2001; Sebbagh et al., 2001). Caspase 3 activity in the transfected myocytes was monitored by counting the number of transfected cells with nuclear localization of fluorescent fusion protein. Overexpression of ROCK1Δ1 induced significant caspase 3 activation and dramatic cell shape changes in transfected myocytes, whereas overexpression of full length ROCK1 had no significant effect compared with control plasmid transfected cardiomyocytes. These results indicate that expression of the truncated active ROCK1 is sufficient to induce activation of the caspase cascade.

### Example 7

### Caspase 3 Activation in Failing Hearts Results in SRF Cleavage

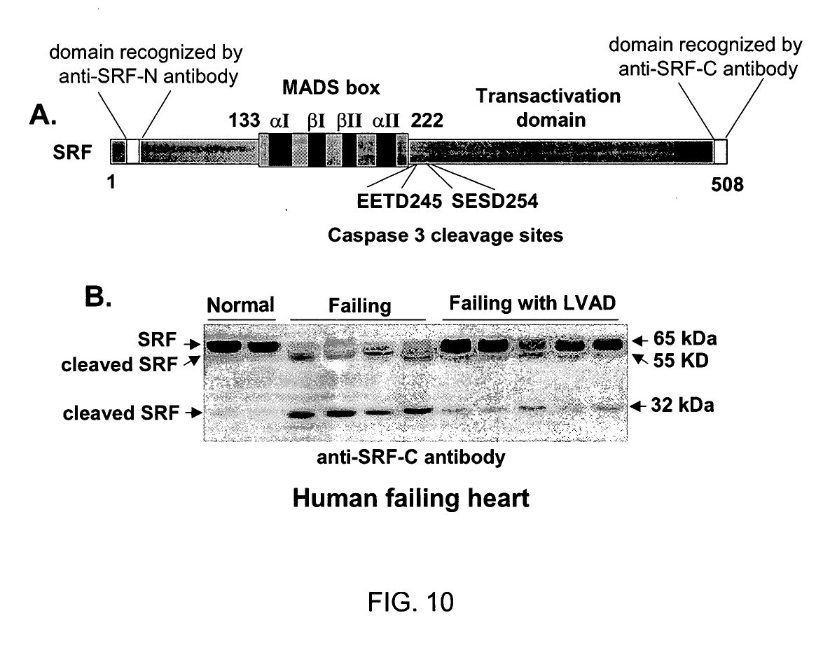

SRF is cleaved by caspase 3 in human heart failure thus generating a dominant negative transcription factor (Chang et al., 2003). Two recent studies have shown that SRF is cleaved by caspase 3 in apoptotic non-cardiac cells (Drewett et al., 2001; Bertolotto et al., 2000). The present inventors examined SRF protein levels from the same cardiac samples as described in above. Full length SRF (65 kDa) was markedly reduced and processed into 55 and 32 kDa fragments in all failing hearts (FIG. 10). In contrast, SRF fragmentation was markedly reduced in failing hearts with LVAD support. Expression of SRF-N, the 32 kDa fragment, in neonatal cardiomyocytes inhibited the transcriptional activity of the cardiac α-actin promoter by 50-60%. The results indicate that modest caspase 3 activation in failing hearts results in cleavage of SRF, which generates a dominant negative transcription factor. Reduced full length SRF levels and the generation of a dominant negative may contribute to the reduced expression of cardiac specific genes.

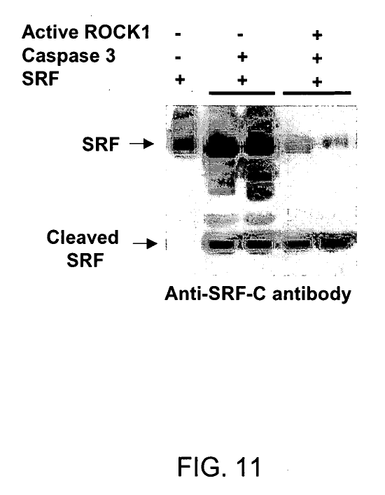

FIG. 11 shows that phosphorylation of SRF by active ROCK1 facilitates SRF cleavage by caspase 3 in vitro. Equal amounts of SRF were incubated with active caspase 3 in the presence or absence of ROCK1Δ1. The full-length level was markedly decreased in the presence of active ROCK1, while the level of the cleaved fragment recognized by the anti-SRF-C antibody was not increased, most likely due to further degradation by caspase 3. In addition, phosphorylation of SRF by ROCK1 did not affect the cleavage sites.

### Example 8

### Phosphorylation of SRF by Rho Kinase Facilitates SRF Cleavage by Caspase 3 In Vitro

Activated caspase 3 is able to cleave SRF in vitro, and phosphorylation of SRF by activated Rho kinase (ROCK1 Δ1) facilitates this reaction (FIG. 11). These in vitro observations indicate an exemplary mechanism involving caspase 3, ROCK1, and SRF in heart failure: activated caspase 3 induces activation of ROCK1, which further facilitates SRF cleavage by caspase 3 through phosphorylation of SRF.

### Example 9

### Cardiac-Specific and Ligand-Inducible Gene-Switch Expression System

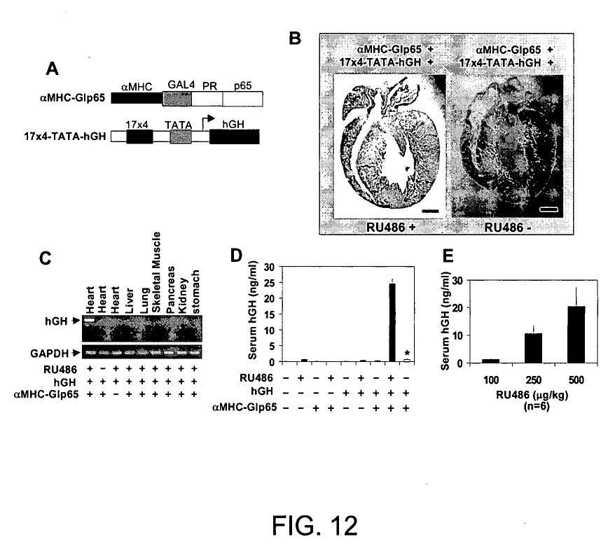

The present inventors have developed a new transgenic strategy aimed to control cardiac-specific expression of transgene in a temporally controlled manner. This ligand-inducible system, named the “gene-switch”, was initially developed by Drs. S. Tsai, F. DeMayo, and B. O'Malley (Wang et al., 1997; Burcin et al., 1999). The present inventors have successfully adapted this system for cardiac-specific expression of human growth hormone (FIG. 12). The inducible level in the transgenic heart is estimated to be over 100 to 1000-fold higher versus the basal level after 4 days of administration of the ligand inducer RU486 in a dose-dependent manner.

### Example 10

### Studies Concerning ROCK1 Activation by Caspase 3 Cleavage Leading to Cardiomyocyte Apoptosis in Cultured Cardiomyocytes

The present inventors have demonstrated that RhoA, Rho kinase and SRF are important regulators of hypertrophic responses to pressure overload. The ROCK1^{-/-} deficient mice exhibit a phenotype that indicates that ROCK1 activation in response to pressure overload potentiates the transition from cardiac hypertrophy to heart failure. In addition, the data in human failing hearts and in apoptotic cultured cardiomyocytes indicates a potential mechanism involving caspase 3-dependent constitutive activation of ROCK1, which in some embodiments of the invention plays an important role in the development of heart failure, in part through facilitating SRF cleavage by caspase 3. These important discoveries lead to the studies of the next Examples concerning the contributory role of ROCK1 and the underlying mechanisms to the development of human heart failure.

The present Example concerns the study of how ROCK1 activation by caspase 3 cleavage leads to cardiomyocyte apoptosis in cultured cardiomyocytes. In a first embodiment of the invention, a caspase 3 cleavage resistant mutant (ROCK1_{D1113A}) or a kinase defective mutant (ROCK1_{KD}) protects cardiomyocytes from apoptosis. In another embodiment of the invention, ROCK1Δ1 induces cardiomyocyte apoptosis through activation of the caspase cascade. In an additional embodiment, ROCK1Δ1 facilitates cleavage of SRF by caspase 3. In a further embodiment, ROCK1Δ1 induces myocyte apoptosis through repressing activity of critical survival signaling pathways.

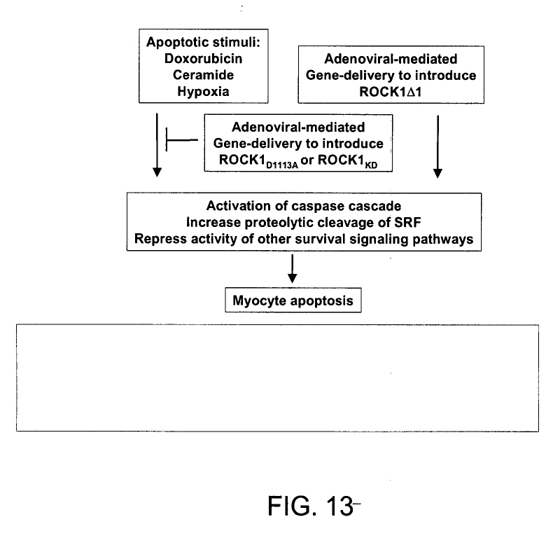

FIG. 13 provides an exemplary diagram of the design of the studies pursuant to embodiments described in this Example. When cultured cardiomyocytes are treated with doxorubicin, a potent apoptotic inducer, ROCK1 is cleaved and the cleavage is inhibited by caspase 3 inhibitors. Moreover, ROCK1 is also cleaved in cultured cardiomyocytes infected with an adenovirus encoding a conditional caspase 3 and in the presence of CID, which activated the conditional caspase 3. These observations indicate that ROCK1 can be cleaved by caspase 3 in apoptotic myocytes. A unique caspase 3 cleavage site on ROCK1, at Asp 1113, removes the C-terminal auto-inhibitory domain resulting in RhoA-independent activation of ROCK1 (Coleman et al., 2001; Ueda et al., 2001; Sebbagh et al., 2001). Expression of a C-terminally truncated mutant (1-1080) of ROCK1 (ROCK1Δ1), which closely mimics caspase 3 cleaved fragment, leads to activation of caspase 3 in cultured cardiomyocytes.

Embodiments described in this Example will characterize the exemplary mechanisms through which activated ROCK1 induces myocyte apoptosis and determines whether activation of ROCK1 by caspase 3 cleavage plays an obligatory role in mediating apoptosis in cultured rat neonatal cardiomyocytes, which are terminally differentiated cardiac cells, and represent a valid cell culture system. In specific embodiments, an adenoviral-mediated expression system is utilized to increase the levels of activated ROCK1 or to express ROCK1 mutants, which inhibit caspase 3-dependent activation of endogenous ROCK1 .

In specific embodiments, cleavage and activation of ROCK1 by caspase 3 serves as an obligatory step for cardiomyocyte apoptosis in response to apoptotic. stimuli in cultured cardiomyocytes. Toward this embodiment, neonatal rat cardiomyocytes are infected with adenovirus encoding the ROCK1 mutant, which is resistant to caspase 3 cleavage (ROCK1_{D1113A}), or a kinase defective mutant of ROCK1 (ROCK1_{KD}) (K105D mutation in the kinase domain that ablates kinase activity) and then challenged by doxorubicin (1-5 μM, 20 h), ceramide (10-50 μ/ml, 20 h) or hypoxia (airtight box saturated with 95% N₂/5% CO₂ for 20 h). Previous studies have shown that the kinase deficient mutant of ROCK1 behaves as a dominant negative to inhibit RhoA-induced stress fiber and focal adhesion formation (Itoh et al., 1999). The mutant ROCK1_{D1113A} is expected to compete with endogenous ROCK1 for cleavage by caspase 3, and the mutant ROCK1_{KD} should behave as a dominant negative for the activated endogenous ROCK1 by caspase 3 cleavage. The present inventors have already constructed an exemplary adenoviral vector for ROCK1_{KD}, which contains a Myc-epitope. In specific embodiments, an adenovirus for ROCK1_{D1113A} (such as, for example, from using AdEasy system from Stratagene) is generated.

After adenoviral transduction of ROCK1 mutants into cardiomyocytes, one will then examine expression of ROCK1 mutants by Western blotting using anit-Myc or anti-ROCK1 antibody (Santa Cruz). Rho kinase activity is assayed, for example by Western blot analysis using the anti-Thr 445-phosphorylated α-adducin antibody (Santa Cruz) and anti-phospho-MLC antibody (New England Biolabs). The same blots are reprobed with an anti-MLC as an equal charge control. Cardiomyocyte apoptosis is assayed by TUNEL staining (immunostaining with Apoptag system from Intergen), DNA fragmentation (DNA laddering with agarose gel electrophoresis), and sub-G1 phase DNA (flow cytometry at the BCM Flow Cytometry Core Facility). In specific embodiments, cardiomyocyte apoptosis induced by treatment with by doxorubicin, ceramide or hypoxia is significantly suppressed in the presence of Ad-ROCK1_{D1113A} or Ad-ROCK1_{KD}, but not with control virus (Ad-β-Gal).

In other embodiments of the present invention, activated ROCK1 induces cardiomyocyte apoptosis through activation of the caspase cascade. To further characterize the mechanisms by which activated ROCK1 stimulates apoptosis, activated ROCK1 is overexpressed in cardiomyocytes using an adenovirus-mediated delivery system. Adenoviral vectors for ROCK1Δ1 and ROCK1 Δ1_{KD} (with a N-terminal Myc-epitope) may be generated. Adenoviral vector for full length ROCK1 has been constructed. In some embodiments, transduction of Ad-ROCK1 Δ1, but not Ad-ROCK1 Δ1_{KD}, or Ad-ROCK1, leads to cardiomyocyte apoptosis in a dose-dependent manner. Transduction of Ad-ROCK1 at high doses may lead to partial cleavage of ROCK1.

Cardiomyocytes transfected with expression vector encoding ROCK1 Δ1, but not full length ROCK1, exhibited significant activation of caspase 3 compared to vector control transfected myocytes (FIG. 9). In specific embodiments, the level of the activated caspase 3 in cardiomyocytes is assayed infected with Ad-ROCK1 Δ1 by Western blotting using anti-caspase 3 antibody, recognizing both the caspase 3 precursor and active caspase 3 (Santa Cruz). Caspase 3 activity will also be assayed by measuring the proteolytic cleavage of a specific fluorogenic substrate of Ac-DEVD-AMC for caspase 3. (Promega). To examine whether activation of the caspase cascade mediates ROCK Δ1-induced myocyte apoptosis, cardiomyocytes are treated with caspase 3 inhibitor, Z-VAD-fink (BD PharMingen), together with adenoviral transduction of ROCK1 Δ1.

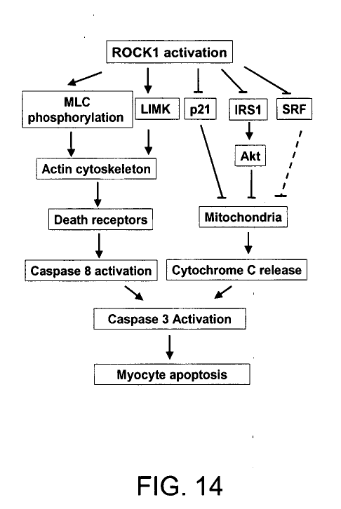

FIG. 14 shows one embodiment of exemplary signaling pathways mediating ROCK1-induced myocyte apoptosis. Activation of ROCK1 may induce the assembly of the death-inducing signaling complex via regulation of actin cytoskeletal rearrangement (mediated by myosin light chain phosphatase, myosin light chain and LIM kinase). Activation of ROCK1 in specific embodiments represses other survival signal pathways, which regulate the mitochondrial pathway (mediated by phosphorylation of insulin receptor substrate 1 (IRS1), repressed expression of p21 and enhanced cleavage of SRF). The role of SRF in regulating mitochodrial pathway (broken line) is suggested by the reported study, which identified MCL1, a Bcl2-related survival factor, as an SRF target gene (Townsend et al., 1999).

Effects of ROCK1Δ1 on the intrinsic mitochondrial pathway is evaluated by measuring: 1) the levels of cytoplasmic fraction of cytochrome c by Western blotting with antibody from PharMingen in cardiomyocytes infected with Ad-ROCK1 Δ1; 2) the levels of Bcl2 (anti-apoptotic) and Bax (proapoptotic), which modulate the release of mitochondrial proteins, by Western blotting with anti-Bcl2 and anti-Bax (Santa Cruz); and/or 3) the activities of JNK and p38 MAP, which are two terminal MAP kinases implicated in cardiomyocyte apoptosis. JNK is also involved in regulating the release of mitochondrial proteins through phosphorylation of anti-apoptotic Bcl2-related proteins, leading to their inactivation or degradation (Tournier et al., 2000; Lei et al., 2002). In specific embodiments, the activity of JNK and p38 is followed by Western blotting with antibodies against JNK, phospho-JNK, p38, phospho-p38 (Cell Signaling).

In specific embodiments, effects of ROCK1 Δ1 on the extrinsic membrane death receptor pathway are evaluated by measuring the level of the activated caspase 8 by Western blotting using anti-caspase 8 antibody recognizing both the caspase 8 precursor and active caspase 8 (PharMingen). Caspase 8 activity is also assayed by colorimetric assay using cell extracts and chromophore p-nitoaniline (p-NA) labeled caspase 8 substrate Ac-IETD-pNA (Clontech).

In addition, the present inventors have observed that ROCK1 Δ1 exhibited increased nuclear localization compared with the full length ROCK1 in transfected CV1 cells (data not shown). By immunostaining and Western blot analysis of nuclear and cytosolic extracts of cardiomyocytes infected with Ad-ROCK1 Δ1 or Ad-ROCK1, it is determined whether ROCK1 Δ1 exhibits increased nuclear translocation compared with full-length ROCK1.

In other embodiments, activated ROCK1 facilitated cleavage of SRF by caspase 3. It was observed that SRF was cleaved by caspase 3 in failing human hearts (Chang et al., 2002). In addition, the activated caspase 3 was able to cleave SRF in vitro, and phosphorylation of SRF by ROCK1 Δ1 facilitated this reaction (FIG. 11). However, in cultured neonatal cardiomyocytes treated with doxorubicin or infected with an adenovirus encoding a conditional caspase 3 (Ad-G/iCasp3) and in the presence of CID (condition for direct activation of caspase 3), significant cleavage of SRF was not detectable (data not shown). In one specific embodiment, the time window for cardiomyocyte apoptosis in the cultured system is either too narrow (less than 20 h) and/or the level of SRF phosphorylation by activated endogenous protein kinases is too low that not enough SRF cleavage could be detected before the final steps of apoptotic reaction (nuclear chromatin condensation and DNA fragmentation) occur.

Adenoviral delivery of ROCK1 Δ1 into cultured cardiomyocytes in specific embodiments substantially increases the level of SRF phosphorylation and produces detectable cleavage of SRF. Phosphorylation level of SRF is measured by immunoprecipitation of SRF using rabbit polyclonal anti-SRF-C antibody followed by Western blotting using mouse monoclonal anti-phosphoserine and anti-phosphothreonine antibodies (Sigma). The effect of SRF phosphorylation on its cleavage by caspase 3 is evaluated with and without co-infection with Ad-G/iCasp3 (plus CID induction).

In addition, SRF is preferentially localized in the nuclei in cardiomyocytes, while ROCK1 Δ1 has both cytosolic and nuclear localization, in specific embodiments. To increase nuclear localization of ROCK1 Δ1, in embodiments wherein it is needed, three SV40 nuclear translocalization signals (NLS) are added to ROCK1 Δ1, for example. When nuclear localization of NLS-ROCK1 Δ1 is demonstrated after adenoviral transduction, phosphorylation and cleavage of endogenous SRF by caspase 3 is assayed as described above. In embodiments wherein no changes in SRF phosphorylation and cleavage can be detected after adenoviral delivery of ROCK1 Δ1 (or NLS-ROCK1 Δ1) together with caspase 3, an alternative embodiment would be that additional apoptotic events are required to induce efficient cleavage of SRF. In this embodiment, cardiomyocyte apoptosis is induced by hypoxia, ceramide, or doxorubicin and studies are performed under these conditions.

In particular aspects of the invention a TAT-mediated protein delivery system (Green and Loewenstein, 1988; Frankel and Pabo, 1988) is utilized to express SRF mutants in cell culture systems. TAT-SRF and TAT-SRF245A/254A (resistant to caspase 3 cleavage) are generated, for example. Under the conditions where significant cleavage of endogenous SRF is detectable (possibly adenoviral delivery of ROCK1 Δ1 with or without low dose caspase 3), it is examined whether introduction of SRF mutant resistant to caspase 3 cleavage inhibits or delays myocyte apoptosis.

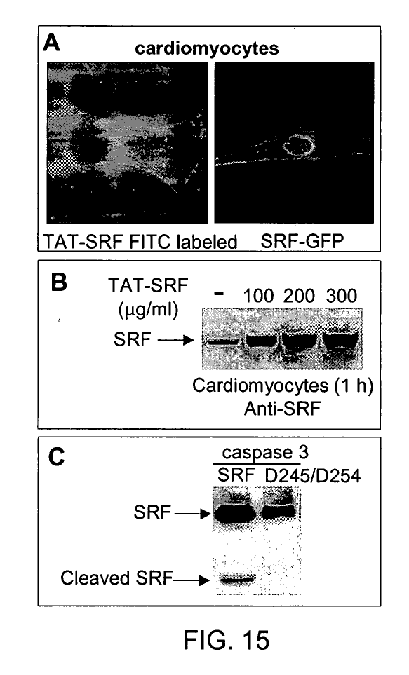

FIGS. 15A and 15B show that TAT-SRF is able to enter into cultured cells in a concentration-dependent fashion and is preferentially localized in the nucleus of cardiomyocytes. In FIG. 15A, TAT-SRF was labeled with FITC and added into the culture medium of neonatal cardiomyocytes. SRF-GFP was expressed through a mammalian expression vector transfected into neonatal cadiomyocytes. TAT-SRF displayed same cellular localization as SRF-GFP and endogenous SRF. In FIG. 15B, Western blot analysis of cardiomyocytes incubated with TAT-SRF at increasing concentrations is provided. Anti-SRF recognizes both endogenous SRF and TAT-SRF, which have similar molecular weight. In FIG. 15C, SRF245A/254A mutant was resistant to caspase 3 cleavage. Purified TAT-SRF and TAT245A/254A were incubated with recombinant caspase 3 in vitro and only TAT-SRF was cleaved by caspase 3.

In another embodiment of the invention, activated ROCK1 represses other survival signal pathways. Many lines of evidence show that the PI3-kinase/Akt signaling pathway plays a key role in regulating cardiomyocyte growth and survival (Matsui et al., 2003). Both insulin-like growth factor (IGF)-1 and IL6-like cytokines induce anti-apoptotic signals in cardiomyocytes through activation of PI3-kinase/Akt pathway (Fujio et al., 2000; Mehrhof et al., 2001; Negoro et al., 2001). Recently, ROCK2 (the major isoform in vascular smooth muscle cells) was shown to bind to the insulin receptor substrate (IRS)-1 resulting in IRS-1 serine phosphorylation that led to inhibition of insulin-induced PI3-kinase activation (Begum et al., 2002; Farah et al., 1998). These observations suggest that activation of ROCK1 by apoptotic cascade inhibits PI3-kinase/Akt pathway through phosphorylation of IRS-1 in cardiomyocytes, in some embodiments.

Phosphorylation of Akt is a hallmark of the activation of the survival pathway directed by IGF-1. To study ROCK1 activation leading to changes in Akt expression and phosphorylation, Akt phosphorylation and protein level is determined by Western blotting with antibodies against Akt and phosphorylated Akt (phospho-Ser473 or phospho-Thr308, Cell Signaling) in cardiomyocytes infected with Ad-ROCK1 Δ1. A critical downstream target of Akt is GSK-3β, which Akt inactivates by phosphorylation (Hardt and SAdoshima, 2002). Phosphorylation of GSK-3β is examined by Western blotting with antibody against phospho-Ser9 (Cell Signaling).

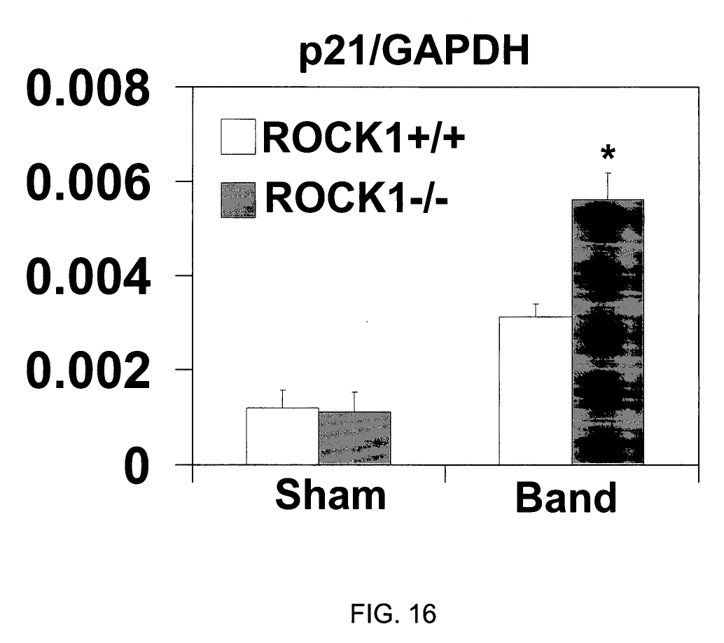

Wide-spread evidence indicates that p21 can play an anti-apoptotic role in various types of cells (Gartel and Tyner, 2002). Exogenous p21 blocks hypoxia-induced cardiomyocyte apoptosis and the anti-apoptotic effects of p21 appear to be independent of Cdk inhibition by p21 (Hauck et al., 2002). RhoA has been reported to repress the expression of p21 in NIH3T3 fibroblasts (Olson et al., 1998). Up-regulation of p21 in the Rho GDIα embryonic hearts of high-copy lines was observed, indicating that Rho family proteins repress p21 expression during early cardiac development (Wei et al., 2002). Rho kinase has been found to repress induction of p21 through inhibiting nuclear translocation of phospho-ERK in phorbol ester-induced apoptotic erythromyeloblast D2 cells (Lai et al., 2002). These observations indicated that activation of ROCK1 by caspase 3 cleavage represses expression of p21 in cardiomyocytes. Consistent with this exemplary embodiment, p21 transcript levels were higher in ROCK1-deficient hearts compared with control mice under pressure overload (FIG. 16).

To determine if activated ROCK1 represses p21 expression in cardiomyocytes, the expression of p21 is followed at both mRNA (real-time RT-PCR) and protein (anti-p21 from Santa Cruz) levels in cardiomyocytes infected with Ad-ROCK1Δ1. In some specific embodiments, transcription level of p21 is repressed by ROCK1 Δ1, but not by ROCK1 Δ1_{KD}. Cellular localization of phospho-ERK is also examined by immunostaining with anti-phospho-p44/p42 ERK antibody (Cell Signaling Technology), for example. It is examined whether exogenous p21 inhibits cardiomyocyte apoptosis induced by ROCK1 Δ1.

In particular embodiments of the present invention, caspase 3-dependent activation of ROCK1 is required in mediating cardiomyocyte apoptosis induced by doxorubicin, ceramide or hypoxia, and that activated ROCK1 is sufficient to induce cardiomyocyte apoptosis. In specific embodiments of the invention, activated ROCK1 induces cardiomyocyte apoptosis by, for example: 1) activation of caspase 8 via the assembly of the death-inducing signaling complex induced by excessive phosphorylation of MLC; 2) release of cytochrome c from mitochrondria into the cytoplasm via inhibition of Akt activity or repression of p21 expression or activation of JNK; and/or 3) increased cleavage of caspase 3 targets including SRF. As the activity of each exemplary mechanism embodiment can be modulated using adenovirus transduction, TAT-mediated protein delivery or specific chemical inhibitors, their cause/effect relationship in mediating ROCK1 effects in cardiomyocyte apoptosis is easily tested.

The designed experiments take advantage of the available information on signaling pathways modulating the activity of apoptotic cascades in cardiomyocytes and other cell types. It is worth noting that other apoptotic or survival signaling pathways (not listed above) may also play a role in mediating pro-apoptotic effects ROCK1 in cardiomyocytes, including, for example calcineurin/NFAT (Pu et al., 2003; Liang et al., 2003), gp130 (Hirota et al., 1999; Jacoby et al., 2003), PKC (Sabri and Steinberg, 2003; Chen et al., 2001; Bueno and Molkentin, 2002; Dom, 2002), etc.

### Example 11

### Studies Concerning ROCK1 Activation by Caspase 3 Cleavage Leading to the Progression of Heart Failure, Which will be Tested Through an Inducible Bi-Transgenic Gain-of-Function Approach

The present example concerns ROCK1 activation by caspase 3 cleavage leading to heart failure progression, which in specific embodiments is tested through an inducible bi-transgenic gain-of-function approach. In specific embodiments, cardiac-specific inducible expression of ROCK1 Δ1 induces cardiomyocyte apoptosis and heart failure in intact animals.

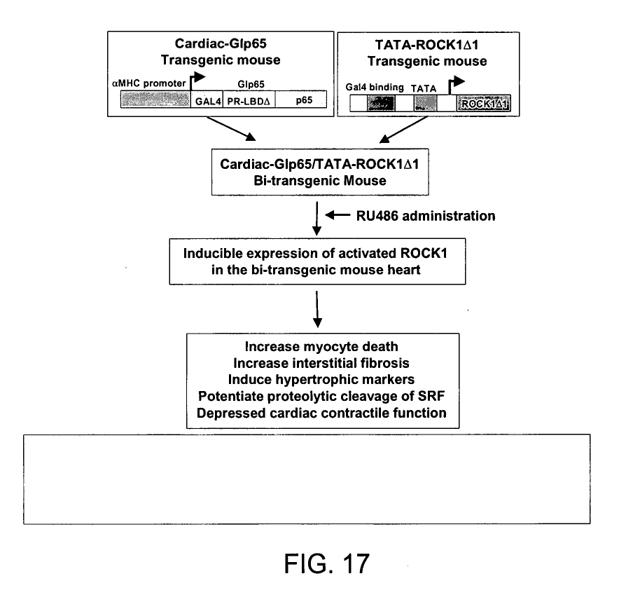

FIG. 17 provides an exemplary embodiment for the present Example. The Examples above indicate that ROCK1 can be cleaved by caspase 3 in apoptotic myocytes and in failing hearts. Expression of activated ROCK1 mutant (ROCK1 Δ1), which closely mimics the caspase 3 cleaved form, leads to the activation of caspase 3 in cultured cardiomyocytes. Hence, in specific embodiments, activation of ROCK1 plays an important role in the progression of heart failure in the intact animal. ROCK1-deficient mice under pressure overload exhibits increase cell size, but there is reduced induction of hypertrophic markers, reduced interstitial fibrosis and reduced apoptosis in comparison with control mice, supporting this embodiment. To determine to what extent the caspase 3-dependent activation of ROCK1 contributes to heart failure, it is helpful to temporally modulate the level of activated ROCK1 in adult heart to avoid large-scale myocyte apoptosis. To achieve this goal, the inducible gene-switch system is used to temporally control cardiac-specific expression of ROCK1 Δ1, by addition of a ligand, RU486.

Cardiac-Glp65/ROCK1 Δ1 bi-transgenic mice are generated. Two exemplary different transgenic lines may be utilized for cardiac inducible expression of ROCK Δ1: 1) The activator line (Cardiac-Glp65) in which the ligand (RU486)-inducible transactivator (Glp65) is under the control of the cardiac-specific αMHC promoter. Glp65 contains a truncated progesterone receptor (PR-LBDΔ), yeast Gal4 DNA binding domain (GAL4) and p65 NF□B transactivation domain (p65); 2) The inducible line (TATA-ROCK1 Δ1) in which the expression of ROCK1 Δ1 is under the control of the promoter (containing four copies of Gal4 binding sites) that can only be activated by Glp65 in the presence of RU486.

Constructs for Inducible-ROCK1 Δ1 have been made and TATA-ROCK1 Δ1 founder mice are generated. The F1 heterozygotes derived from the Inducible-ROCK1 Δ1 founder mice are then crossed with the Cardiac-Glp65 line to generate bi-transgenic mice: Cardiac-Glp65/TATA-ROCK1 Δ1. Cardiac expression of ROCK1 Δ1 is examined by RT-PCR and Western blot analysis (ROCK1 Δ1 contains a Myc epitope) before and after RU486 administration (500 μg/kg body weight) for 4 days (established conditions for maximal level induction of the transgene expression in bi-transgenic hearts) to select transgenic lines with inducible transgene expression. These doses are tolerated in mice, even with long term use. Two bi-transgenic lines showing the highest and lowest range of inducible expression of ROCK1 Δ1 are focused on initially.

As a control for potential non-specific effects of the overexpression of the transgene, an additional bi-transgenic mouse line will also be generated that expresses ROCK1 Δ1_{KD}, a kinase defective transgene. Under basal condition where endogenous ROCK1 activity is not stimulated, inducible expression of ROCK1 Δ1_{KD} in the heart of bi-transgenic mice should not have significant dominant negative effect.

In specific embodiments, inducible expression of ROCK1 Δ1 leads to cardiac hypertrophy and/or heart failure. The effects of the inducible expression of the transgene from 4 weeks of age are determined, for example. Bi-transgenic hearts in specific embodiments have no structural and functional abnormalities in the absence of RU486 administration. For RU486 administration, mice are implanted with RU486 pellets (Innovative Research of America), which are designed for release of the drug at a constant daily dose of 100, 250 and 500 μg/kg body weight for 60 days. Mice will be examined after administration of RU486 for various periods of times (1 to 60 days if compatible with life). Effects of the inducible expression of activated ROCK1 Δ1 on the myocardium are analyzed at molecular, cellular, morphological and functional levels under basal physiological conditions.

Apoptosis: Assay of Rho kinase activity, evaluation of apoptosis by measuring caspase 3 activity, DNA laddering, and sub-G1 phase DNA are performed as described above. In addition, the effects of acute and chronic ROCK1 activation on the regulation of other components of apoptosis machinery in the hearts of bi-transgenic mice are examined. These include the levels of cytoplasmic fraction of cytochrome c, the levels of anti-apoptotic Bcl2 and pro-apoptotic Bax, activation of MAK kinases (ERK, JNK and p38), and caspase 8 activity as described elsewhere herein. Effects of acute and chronic ROCK1 activation on the cleavage of SRF, expression of p21, and activity of PI3-kinase/Akt pathway are also examined as described above.

Both frozen and paraffin sections are made from hearts before and after inducible expression of ROCK1 Δ1. Inducible expression of ROCK1 Δ1 in myocardium leads to significant cardiomyocyte damage, in specific embodiments. Evaluation of cardiomyocyte apoptosis in tissue sections is performed by TUNEL staining using the ApopTag in situ apoptosis detection kit (Intergen), for example, and counter-staining for α-sarcomeric actin. Immunostaining images are captured by Zeiss LSM 510 confocal microscopy with triple laser system, which is available in the Integrated Microscopy Core at BCM. Utrastructural changes are also evaluated, such as chromosomal nuclear condensation and myofibrillar disarray by electron microscopy analysis.

Hvpertrophy: Heart weight/body weight, lung weight/body weight and liver weight/body weight are measured. Myocyte size is determined by measuring the transnuclear width at the mid-ventricular level of hematoxylin-eosin stained heart sections. The longitudinal length of ventricular myocytes is measured using isolated myocytes. Myocyte density in the left ventricular myocardium is determined histologically from hematoxylin-eosin stained heart sections. Changes in the expression profiles of hypertrophic markers including ANF, βMHC, αMHC, skeletal α-actin, BNP, and SERCA2a are measured by real-time RT-PCR.

Fibrosis: The extent of interstitial fibrosis is determined by Sirius Red staining, which is a useful measure of fibrillar collagen, and is coupled with image analysis for quantitation. Expression level of type I and type III collagens is examined by real-time RT-PCR to further quantitate fibrosis, for example.

Gene Expression: In addition to the candidate gene approach, systematic gene profiling is also employed by microarray analysis using Affymetrix mouse gene chips (430 microarray series containing over 34,000 known genes and ESTs) available at the Informatics Microarray Core at BCM. Other mouse chips are also available at Baylor Microarray Core Facility, including mouse 15K and 7.4K arrays based on the 15, 000 and 7,400 cDNA clone sets from the National Institute of Aging. The Baylor Microarray Core Facility also offers Affymetrix services including RNA quality control, Affymetrix labeling, hybridization, and basic or extended data analysis.

Replicate samples for each condition are performed for microarray analysis. For data analysis, the Core facility provides Affymetrix MicroArray Suite 5.0, GeneSpring, and Spotfire. The present inventors also may utilize dChip (Harvard University), BIOConductor (Harvard Medical School), and GenMAPP (UCSF). These software packages are used to cluster patterns of gene expression with similar fold changes compared to a control group.

Among the groups of genes whose expression is significantly altered due to induced expression of ROCK1Δ1, in specific embodiments the following may be found 1) common markers of cardiac hypertrophy; 2) molecular markers for fibrosis including collagens, matrix metalloproteinases and tissue inhibitors of metalloproteinases; 3) SRF gene targets; and/or 4) regulators of mitochondrial apoptotic pathway, etc. The results of microarray analysis are confirmed by quantitative RT-PCR. By comparing gene expression profiling of acute versus chronic induction of ROCK1Δ1, this strategy is aimed at selecting primary ROCK1-regulated genes from targets whose expression may be altered secondary to cardiac remodeling.

In particular embodiments, the data is interpreted in comparison to published (Hwang et al., 2002; Barrans et al., 2002; Tan et al., 2002; Peng et al,. 2002; Aronow et al., 2001; Schneider and Schwartz, 2000) or publicly deposited genome-wide expression profiles, such as Cardio Genomics (www.cardiogenomics.med.harvard.edu) to see if acute or chronic activation of ROCK1 triggers gene profile changes similar or different compared to other cardiac hypertrophy and/or heart failure conditions.

Systolic and diastolic function: Cardiac performance of bi-transgenic mice is evaluated before and after inducible expression of ROCK1Δ1 by non-invasive measurements of systolic and diastolic function with pulsed wave Doppler-echocardiograpy. The measurements include left ventricular end-diastolic and end-systolic diameters, left ventricular end-diastolic wall thickness and Doppler estimates of stroke volume and cardiac output.

It is first determined if acute induction of high level or chronically low-level expression of ROCK1Δ1 induces compensatory cardiomyocyte hypertrophy or decompensated dilated cardiomyopathy. Based on the results of the present inventors, in some embodiments under either condition, increased mortality, dilated cardiomyopathy, increased frequency of myocyte apoptosis, increased interstitial fibrosis, decreased myocyte density, pathological changes of expression profiles of hypertrophic markers, activation of caspase 3, activation of other components of apoptotic cascade, repression of p21, decreased activity of PI3-kinase/Akt cascade, and/or cleavage of SRF is determined.

In specific embodiments, the severity of these effects correlates with the level and duration of induced expression of ROCK1 Δ1. The comparison between acute (less than 7 days) and chronic (up to 60 days) activation of ROCK1 allows one to distinguish between the primary and secondary effects caused by this transgene, and to determine the contribution of ROCK1 activation in the initiation and progression of heart failure. As the gene-switch system allows the present inventors to turn on and off the transgene, the consequences of withdrawal of ROCK1 Δ1 expression can be determined. In specific embodiments, this determines to what extent injury caused by ROCK1 activation can be reversed. However, inducible expression of ROCK1 Δ1 in specific embodiments irreversibly activates the caspase cascade so that withdrawal of ROCK1 Δ1 expression cannot stop the apoptotic process.

### Example 12

### Studies Concerning the Role of ROCK1 in Mediating Heart Failure Uunder Cardiac Conditions Associated with Caspase 3 Activation, Using ROCK1-Deficient Mice, Cardiac-Specific ROCK1-Deficient Mice, and Mice with a Knockin Mutation in the ROCK1 Gene Resistant to Caspase 3 Cleavage

The present Example relates to the role of ROCK1 in mediating heart failure under cardiac conditions associated with caspase 3 activation, using ROCK1-deficient mice, cardiac-specific ROCK1-deficient mice, and mice with a knockin mutation in the ROCK1 gene resistant to caspase 3 cleavage. In specific embodiments, ROCK1 deficiency inhibits cardiomyocyte apoptosis and heart failure under the pathological conditions in which apoptosis plays a significant role in the development of heart failure. In another embodiment, the in vivo knockin mutation of the endogenous ROCK1, resistant to caspase 3 cleavage, inhibits cardiomyocyte apoptosis and heart failure under these conditions.

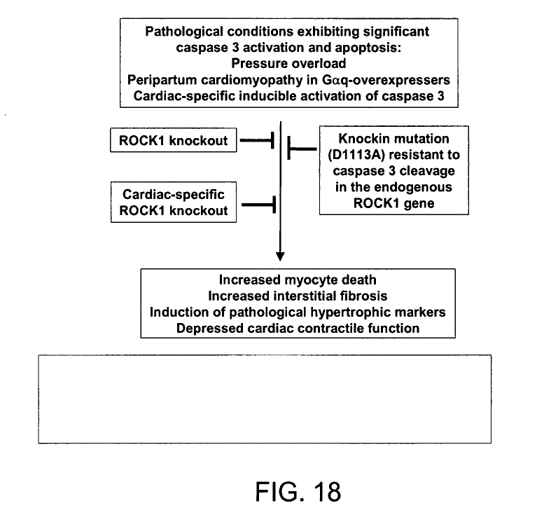

An exemplary study design is provided in FIG. 18. The Examples above indicate that activation of ROCK plays an important role in the progression of heart failure in the intact animal. The gain of function studies described in Example 11 will address activation of ROCK1 as being sufficient to cause heart failure. However, further studies will address activation of endogenous ROCK1 in mediating cardiomyocyte apoptotic signals in the intact animal by loss-of-function approaches. In specific embodiments, endogenous ROCK1 can be activated by two exemplary pathways: 1) RhoA-dependent; and 2) RhoA-independent and caspase 3-dependent. ROCK1 deficiency abolishes the activation by both pathways and the ROCK1 mutation, resistant to caspase 3, only abolishes one pathway. It is tested if ROCK1 plays an obligatory role in heart failure progression by three parallel approaches: 1) ROCK1 knockout mice, in which deficiency of ROCK1 in both myocardium and vascular system contributes to the phenotype; 2) ROCK1 conditional knockout mice, in which only the role of ROCK1 in cardiomyocytes is studies; and/or 3) mice with a knockin mutation causing resistance to caspase 3 cleavage in the endogenous ROCK1 gene, in which only the role of caspase 3-dependent activation of ROCK1 is studied. To induce cardiomyocyte apoptosis in the intact animal, three conditions are employed: 1) biomechanical stress generated by transverse aortic banding; 2) Gαq-mediated peripartum cardiomyopathy; 3) direct activation of caspase 3 by inducible expression and activation of caspase 3 in the heart. The first two model embodiments are established pathological conditions under which significant apoptosis has been documented.

In some embodiments, ROCK1 is activated by pressure overload, in Gαq transgenic hearts or by inducible activation of caspase 3. RhoA and Rho kinase expression and/or activity are up-regulated in rat hearts under pressure overload (Torsoni et al., 2003), in the hypertrophic hearts of Dahl salt-sensitive hypertensive rats (Kobayashi et al., 2002; Satoh et al., 2003) and in the hypertrophic hearts of angiotensin II-infused rats (Higashi et al., 2003). However, relative ROCK1 and ROCK2 activation was not examined.

In embodiments to examine whether RhoA/Rho kinase signal pathway is activated in vivo, ROCK1 and ROCK2 mRNA levels are measured by real-time RT-PCR; by the protein levels by Western blotting, which also detects the cleaved fragment of ROCK1; and kinase activity by measuring the phosphorylation level of ROCK1 and ROCK2 endogenous substrates as described above.

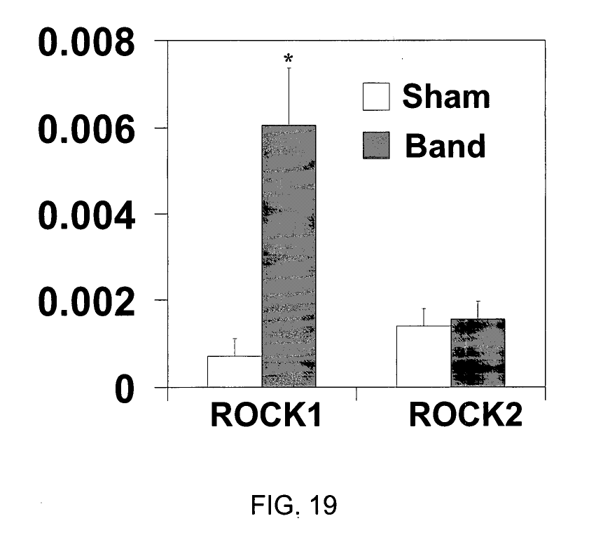

Pressure overload condition is addressed in FIG. 19. By real-time RT-PCR analysis, it was observed that ROCK1 transcript levels were up-regulated in hypertrophic hearts induced by constricting the transverse aorta for three weeks, while ROCK2 transcript levels remained unchanged (FIG. 19). To determine the time course of up-regulation of ROCK1 expression by pressure overload, the expression of ROCK1 . ROCK2 and Rho kinase activity at 24 h, 1 week, 2 weeks and 3 weeks of banding is identified.

Peripartium Gαq transgenic hearts. Gαq-overexpressing mice develop a compensated cardiac hypertrophy under basal conditions. Apoptotic decompensation is observed in peripartium Gαq transgenic hearts. The peak incidence of heart failure occurs within 1 week after delivery (Adams et al., 1998). The expression of ROCK1, ROCK2 and Rho kinase activity is determined in hypertrophic hearts (12 week-old of Gαq mice) as well as in peripartium cardiomyopathic hearts.

Inducible activation of caspase 3 mice is addressed. In both pressure overload and peripartium Gαq transgenic hearts, endogenous ROCK1 and ROCK2 can be activated by a RhoA-dependent pathway and ROCK1 can be activated by a caspase 3-dependent pathway. ROCK1 activation by caspase 3 cleavage may not be a major determinant of increased Rho kinase activity under these conditions. In a mouse model that exhibits increased caspase 3 activity independent of other hypertrophic or apoptotic responses, caspase 3-dependent cleavage of ROCK1 is a major contributor to increased Rho kinase activity, in some embodiments.

A bi-transgenic inducible model aimed at temporally modulating caspase 3 activity in the heart is generated, in some embodiments. In this model, expression of a conditional caspase 3 (iCasp3) is under the control of RU486 through the gene-switch system described elsewhere herein. This conditional caspase 3 remains in its inactive form unless forced to dimerize by addition of a CID, AP20187. Using this sophisticated regulatory system, the activity of transgenic caspase 3 is controlled at both transcriptional (by RU486) and post-translational (by CID) levels by addition of two ligands in a dose-dependent manner.

The constructs for TATA-iCasp3 have been made, and 7 positive TATA-iCasp3 founders were generated. The F1 heterozygotes from these founders are being bred with the Cardiac-Glp65 line to generate bigenic mice: Cardiac-Glp65/iCasp3, which is then tested for inducible expression and activation of caspase 3 by RU486 and CID administration, and RT-PCR and Western blot analysis. It is tested whether these mice exhibit significant cardiomyocyte apoptosis and develop dilated cardiomyopathy within, for example, the next six months. As part of the characterization of the inducible caspase 3 mice, expression of ROCK1, ROCK2, and Rho kinase activity is examined before and after inducible activation of caspase 3 in the bi-transgenic hearts.

In some embodiments, the essential functions of ROCK1 activation in mediating apoptotic signals are as follows: ROCK1-deficient mice exhibit decreased myocyte apoptosis, decreased interstitial fibrosis and increased induction of pathological hypertrophic markers, and improved contractile function compared to control mice under pressure overload. This embodiment is further characterized as to the importance of ROCK1 in mediating apoptotic signals under pressure overload and other apoptotic decompensated conditions. The characterization of the pathological phenotype is performed as described elsewhere herein.

Pressure overload condition Decreased cardiomyocyte apoptosis has been observed using TUNEL staining and increased fibrosis by Sirius Red staining following aortic banding. To complete this analysis of cardiomyocyte apoptosis, DNA fragmentation, sub-G1 phase DNA, caspase 3 activity, activation of MAK kinases (ERK, JNK and p38), and caspase 8 activity are measured as described elsewhere herein. In specific embodiments, the present inventors will measure expression level of type I and type III collagens by real-time RT-PCR to further analyze fibrosis. The protein level of p21 (to confirm RT-PCR results) and the activity of PI3-kinase/Akt pathway are also examined. To gain further mechanistic insight, the present inventors will also perform microarray analysis to compare gene profile changes between ROCK1 deficient mice and control mice before and after aortic banding.

Peripartium Gαq transgenic hearts. The present inventors are in the process of generating Gαq/ROCK1^{-/-} mice: breeding of Gαq mice with ROCK1^{-/-} mice is performed to obtain Gαq/ROCK1^{-/-} mice (expected in 50% of offspring), which is then bred with ROCK1^{-/-} mice to generate Gαq/ROCK1^{-/-} mice (expected in 25% of offspring). The question of whether ROCK1 deficiency affects Gαq expression is examined, as is the question of whether ROCK1 deficiency causes changes in the characteristic features of cardiac hypertrophy in Gαq transgenic mice under normal conditions. It is then examined if ROCK1 deficiency delays or prevents the early death seen in the peripartum period of Gαq transgenic mice, and if ROCK1 deficiency inhibits cardiomyocyte apoptosis, and whether ROCK1 prevents dilated cardiomyopathy.

Inducible activation of caspase 3 mice. Inducible activation of caspase3 is expected to cause increased mortality, dilated cardiomyopathy, increased frequency of myocyte apoptosis, increased interstitial fibrosis, and depressed cardiac contractile function, in some embodiments. To characterize the role of ROCK1 in mediating cardiomyocyte apoptosis induced by direct caspase 3 activation, inducible caspase 3 transgenic mice are crossed into the ROCK1 knockout background. Cardiac-Glp65 and TATA-iCasp3 mice are then bred with ROCK1^{-/-} mice to generate Cardiac-Glp65/ROCK1^{-/-} and TATA-iCasp3/ROCK1^{-/-} mice, as described above for the generation of Gαq/ROCK1^{-/-} mice. These two lines of mice are then intercrossed to produce Cardiac-Glp65/iCasp3/ROCK1^{-/-} mice (expected in 25% of offspring). Caspase 3 activity is then induced by administration of RU486 and CID, and it is determined to what extent ROCK1 deficiency delays or prevents cardiomyocyte apoptosis and heart failure triggered by acute or chronic activation of caspase 3.

In specific embodiments of the present invention, the cardiomyocyte-autonomous functions of ROCK1 activation in mediating apoptotic signals are determined. During mouse embryonic development, ROCK1 is enriched in the developing heart (Wei et al., 2001), but it is also expressed in other tissues including the developing vascular system. In adult mice, ROCK1 is also abundant in vascular smooth muscle and endothelial cells (Nakagawa et al., 1996). Moreover, in mouse heart, ROCK1 is present in both cardiomyocytes and cardiac fibroblasts. Previous studies using Rho kinase inhibitors support an important role of Rho kinase in regulating contraction of smooth muscle cells, thereby regulating blood pressure (Uehata et al., 1997), repressing eNOS expression in endothelial cells, which has cardioprotective effects (Takemoto et al., 2002), and promoting smooth muscle proliferation (Sauzeau et al., 2001; Sauzeau et al., 2000).

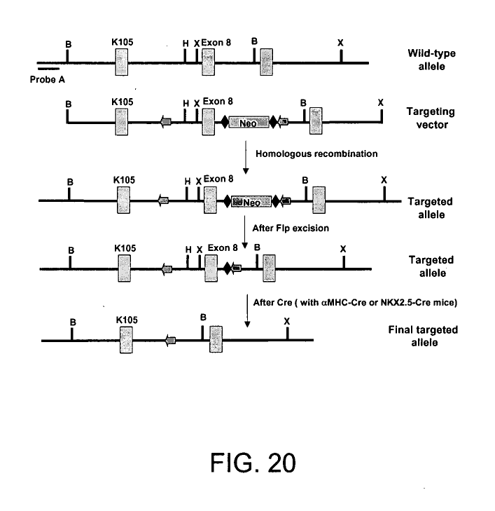

Although previous studies do not distinguish between the role of ROCK1 and ROCK2 in regulating these functions (chemical inhibitors inhibit the activity of both isoforms), these data indicate that ROCK1 deficiency in smooth muscle cells, endothelial cells and cardiac fibroblasts contributes to the decreased apoptosis and interstitial fibrosis observed in ROCK1-deficient mice under pressure overload, in specific embodiments. To specifically study the role of cardiomyocyte ROCK1 in mediating myocyte apoptosis in vivo, mutant mice that lack endogenous ROCK1 in cardiomyocytes are generated, such as through a combined Cre/Flp system. An exemplary strategy is provided in FIG. 20.

Generation of conditional ROCK1 knockout mice. The ROCK1-loxP mice are generated by introducing loxP sites into ROCK1 gene by homologous recombination, in specific embodiments (FIG. 20). To construct the targeting vector, the same genomic ROCK1 DNA clone is used (isolated from an isogenic ES 129 mouse genomic library) that was previously used to generate ROCK1^{-/-} mice. The targeting vector is introduced into ES cells, such as by electroporation, and then placed under G418 selection. Clones with the appropriate recombination event are identified by Southern blot analysis of DNA probing with sequences located outside the targeting vector. Correctly targeted ES cells are injected into C57BL/6 blastocysts. Chimera mice carrying the mutant allele are bred to C57BL/6 mice to generate heterozygous mice, which are then bred to Gt(ROSA)26Sor-Flp mice (such as commercially obtained from The Jackson Laboratory) to delete the Neo cassette from the germ line via the Flp-Frt system (Farley et al., 2000). In Gt(ROSA)26Sor-Flp mice, Flp recombinase is targeted into the constitutive ROSA26 locus. In particular embodiments, this targeting approach results in the generation of ROCK1-loxP mice with loxP sites in the introns flanking the exon 8 encoding residues 137-196. Deletion of this exon by Cre recombinase results in a frame-shift mutation in ROCK1, thus removing all residues from the residue 137 to the end of the protein. In ROCK1 knockout mice, deletion of all residues from the residue 180 to the end of the protein results in a null mutation of ROCK1.

Homozygous ROCK1-loxP (ROCK1^{f/f}) mice are then crossed to the Nkx2.5-Cre mice, generated as described previously (Moses et al., 2001), or to the αMHC-Cre mice generated as described previously (Agah et al., 1997). ROCK1^{f/f} mice are bred with Nkx2.5-Cre or αMHC-Cre mice to obtain ROCK ^{f/+}/Cre mice (50% of offspring expected), which are then bred with ROCK1^{f/f} mice to generate ROCK1^{f/f}/Cre mice (αMHC or Nkx2.5) (expected in 25% of offspring). The approximate percentage of homologous ROCK1 knockouts is assayed by Southern blot analysis of cardiac tissue, PCR analysis, as well as by in situ hybridization and immunohistochemistry. Complete cardiac recombination should be achieved by embryonic day 11-12 (Gaussin et al., 2002).

In a particular embodiment of the present invention, cardiac-specific ablation of ROCK1 decreases cardiomyocyte apoptosis under pathological conditions. In some embodiments, ROCK1^{f/f}/αMHC-Cre or ROCK1^{f/f}/Nkx2.5-Cre are utilized in the following studies. Similar experiments are performed as for ROCK1 deficient mice under the three pathological conditions described above: pressure overload, peripartium Gαq and cardiac-specific inducible caspase 3. It is examined whether the characteristic features (at least some) observed in ROCK1-deficient mice are recapitulated in cardiac-specific ROCK1 knockout mice.

To characterize the effect of cardiac-specific ablation of ROCK1 on peripartium Gαq-mediated dilated cadiomyopathy, Gαq/ROCK1^{f/f}/Cre mice are generated. Gαq/ROCK1^{f/f} mice are first generated through the strategy described above for the generation of Gαq/ROCK1^{-/-} mice. ROCK1^{f/f}/Cre mice are then crossed with Gαq/ROCK1^{f/f} mice to produce Gαq/ROCK1^{f/f}/Cre mice (expected in 25% of offspring).

To characterize the effect of cardiac-specific ablation of ROCK1 on caspase 3-induced cardiomyopathy, Cardiac-Glp65/iCasp3/ROCK1^{f/f}/Cre mice are generated. Cardiac-Glp65/iCasp/ROCK1^{f/f} mice are first generated through the same strategy as described above for the generation of Cardiac-Glp65/iCasp3/ROCK1^{-/-} mice. These mice are then crossed with ROCK1^{f/f}/Cre mice to generate Cardiac-Glp65/iCasp3/ROCK1^{f/f}/Cre (expected in 12.5% of offspring).

In other embodiments, caspase 3-dependent activation of ROCK1 is required for mediating apoptotic signals. The activation of ROCK1 by both RhoA-dependent pathway and RhoA-independent and caspase 3-dependent pathway contributes to the phenotype observed in ROCK1-deficient mice under pathological conditions, in specific embodiments of the invention. To directly characterize the role of caspase 3-dependent cleavage and activation of ROCK1 in mediating myocyte apoptosis in vivo, mutant mice are generated that lack the caspase 3 cleavage site in the endogenous ROCK1 gene.

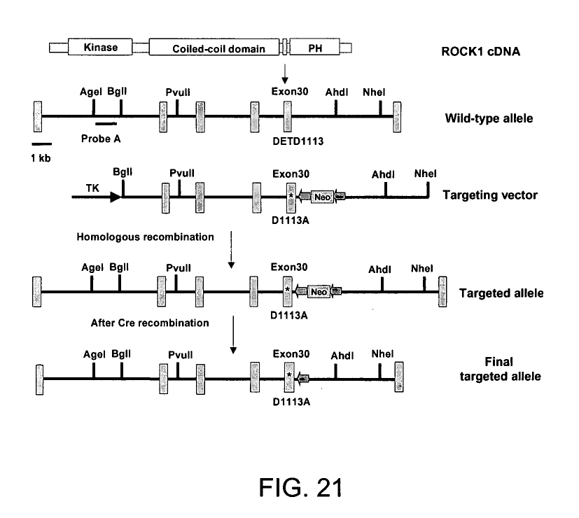

Generation of mice with D1113A knockin mutation of the endogenous ROCK1 gene. The mice are generated by introducing a point mutation at the caspase 3 cleavage site using homologous recombination techniques (FIG. 21). ROCK1 BAC clones are first isolated from a BAC library of the 129 strain. A BAC clone is screened for that contains ROCK1 coding sequences including exon 30 (which contains the caspase 3 clevage site) by PCR and Southern blot analysis. A targeting vector encoding Ala substitution at Asp 1113, in exon 30 is constructed with a loxP-flanked neomyocin resistant maker in the downstream intron (the resistance to caspase 3 cleavage by this point mutation is first tested in cell culture as described above). Correctly targeted ES cells containing Ala mutation and Neo cassette are injected into C57BL/6 blastocysts. Chimera mice carrying the mutant allele are bred to C57BL/6 mice to generate heterozygous mice. Heterozygous mice are then bred to EIIa-Cre mice to delete the Neo cassette from the germ line via the Cre-loxP system (Lakso et al., 1996). In EIIa-Cre mice, the Cre transgene is under the control of the adenovirus EIIa promoter (Lakso et al., 1996).

As an alternative embodiment of conventional targeting approach, one can also introduce the D1113A mutation into the BAC ROCK1 and then introduce the modified BAC clone into ES cells, which are then screened for the appropriate homologous recombination as described above. The advantages of the BAC approach include the significant reduction in time for the generation of the targeting vector. Only a 50 nt homology region flanking the Neo-loxP cassette is required for efficient homologous recombination with the genomic DNA of ROCK1 in the BAC clone in bacteria (Nefedov et al., 2000; Muyrers et al., 1999). FIG. 21 illustrates an exemplary strategy for generation of D1113A knockin mutation mice. The final targeted allele contains the mutated caspase 3 cleavage site and one loxP site within the downstream intron.

In other embodiments of the invention, the elimination of the caspase 3 cleavage site in ROCK1 leads to decreased cardiomyocyte apoptosis under pathological conditions. Studies are performed in ROCK1D1113A mice under three pathological conditions as describe above for ROCK1 deficient mice. It is examined if the characteristic features (at least some) observed in ROCK1 deficient mice are recapitulated in ROCK1D1113A mice.

To test an effect of the absence of caspase 3 cleaved ROCK1 on peripartium Gxq-mediated dilated cadiomyopathy, Gαq/ROCK1_{D1113A} mice are generated through the strategy described above for the generation of Gαq/ROCK1^{-/-} mice. To test an effect of the absence of caspase 3 cleaved ROCK1 on caspase 3-induced cardiomyopathy, Cardiac-Glp65/iCasp3/ROCK1_{D1113A} mice are generated through the strategy described above for the generation of Cardiac-Glp65/iCasp3/ROCK1^{-/-} mice.

In an embodiment of the invention, ROCK1 is activated by RhoA-dependent pathway and by RhoA-independent and caspase 3-dependent pathway. As such, it is beneficial to determine the contribution of each pathway in regulating cardiomyocyte apoptosis in vivo. The observations in cultured cardiomyocytes clearly demonstrate increased toxicity of ROCK1Δ1 versus full-length ROCK1 in inducing activation of caspase 3 (FIG. 9). By comparing the characteristic features of ROCK1-deficient mice with those of ROCK1_{D1113A} mutant mice, in specific embodiments the role of caspase 3-dependent activation of ROCK1 in regulating cardiomyocyte apoptosis in vivo is determined. In one embodiment, activation of ROCK1 (as well as ROCK2) by RhoA pathway plays a beneficial role in the development of compensated cardiac hypertrophy, while activation of ROCK1 by caspase 3-dependent cleavage contributes to the transition from cardiac hypertrophy to heart failure.

As mentioned above, ROCK1 is expressed in cardiomyocytes, cardiac fibroblasts, smooth muscle cells and endothelial cells. By comparing the characteristic features of ROCK1-deficient mice with those of cardiac-specific ablation of ROCK1 mice, one is able to distinguish between the effects of cardiomyocyte ROCK1 deficiency and the effects of ROCK1 deficiency in other cell types on regulating cardiomyocyte apoptosis, interstitial fibrosis formation, and pathological changes of gene expression profiles under pathological conditions. Generation of ROCK1-loxP mice also allows for investigation of specific roles of ROCK1 in these cell types by tissue specific ablation of ROCK1, through crossing with mouse lines expressing other tissue specific Cre recombinases.

The studies described herein focus on the role of ROCK1 in mediating myocyte apoptosis during transition to heart failure. In specific embodiments, ROCK2 cannot compensate for the role of ROCK1 in regulating cardiomyocyte apoptosis. The absence of caspase 3 cleavage site on ROCK2 also indicates that ROCK2 does not mediate the effects of caspase 3. In specific embodiments, this is further characterized through ROCK2 knockout or cardiac-specific ROCK2 knockout mice. A recent report has shown that the majority of ROCK2 deficient homozygous mice died in utero due to defects in the placenta. A small number of ROCK2 knockout mice that survived were born runts without significant cardiac structural abnormalities (Thumkeo et al., 2003). In particular embodiments, both ROCK2 knockout and conditional ROCK2 knockout mice are generated and utilized for characterization of ROCK2 in cardiac development, such as in combination with ROCK1 knockout, and is also used for characterizing role of ROCK2 in postnatal cardiac hypertrophy and heart failure.

Thus, as described herein, biological functions of ROCK1 in cardiomyocyte apoptosis and the transition from compensated hypertrophy to heart failure is described by using several exemplary unique in vivo and in vitro model systems: ROCK1-deficient mice, cardiac-specific ROCK1-deficient mice, and ROCK1 mutant mice with caspase 3 resistant point mutation D1113A in the endogenous ROCK1 gene for in vivo loss-of-function study; cardiac-specific inducible ROCK1Δ1 mice for in vivo gain-of-function study, and a number of adenoviruses and TAT-fusion proteins for gain- and loss-of-function studies in cell culture. The function and mechanisms by which ROCK1 regulates cardiomyocyte apoptosis and cardiac remodeling during transition to heart failure are provided, as are novel and powerful reagents to characterize additional roles of ROCK1 in other biological systems.

### Example 13

### Caspase 3 Activation by ROCKΔ1 was Associated with the Activation of PTEN and Dephosphorylation of AKT

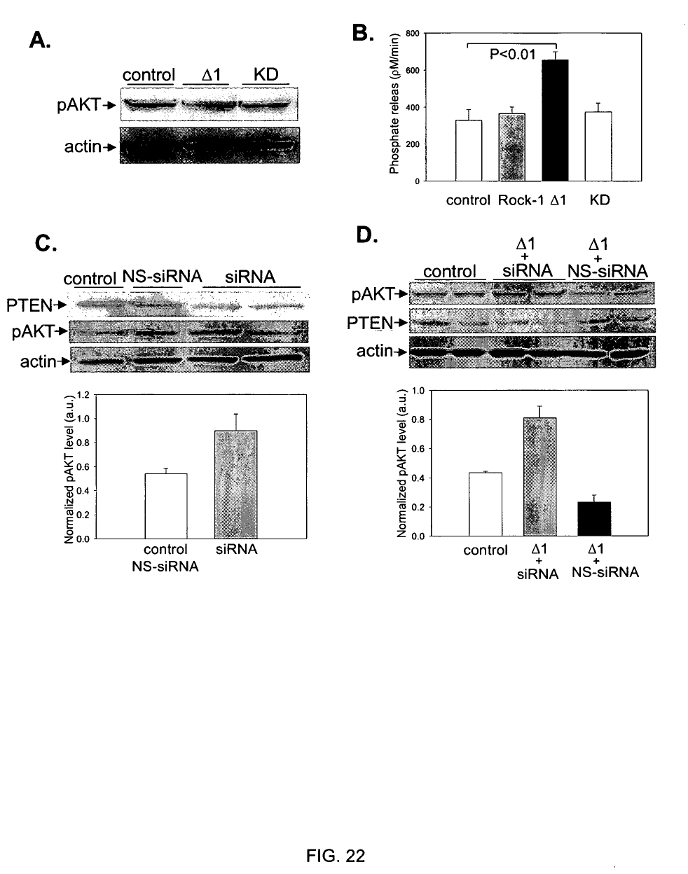

To further characterize the mechanism involved in this caspase activation, the present inventors transfected ROCKΔ1 in human HEK cells. As indicated in FIG. 22, ROCKΔ1 decreased the phospho-AKT (pAKT) level (FIG. 22A) and increased PTEN activity, but this was not found in either full length ROCK-1 or kinase-deficient mutant, ROCK-1KD (FIG. 22B). Since AKT is activated by PI3K, PTEN acts as the upstream PI3K inhibitor. In specific embodiments of the invention, the repression of AKT activity caused by ROCKΔ1 may contribute to its pro-apoptotic effect through the increase in PTEN activity. Loss of PTEN expression by siRNA treatment resulted in an increase in the basal phosphorylation state of AKT (FIG. 22C), which is consistent with other studies (Li et al., 2005; Oudit et al., 2004). Meanwhile, the decreased level of pAKT by ROCKΔ1 was reverted by blocking PTEN expression. Furthermore, the pAKT level was higher than the control level (FIG. 22D), which further supported this specific embodiment.

### Example 14

### Deficiency of ROCK-1 Prevented Apoptosis Induced by Ceramide in Cultured Cardiomyocytes or in Mice Subjected to Systolic Overload

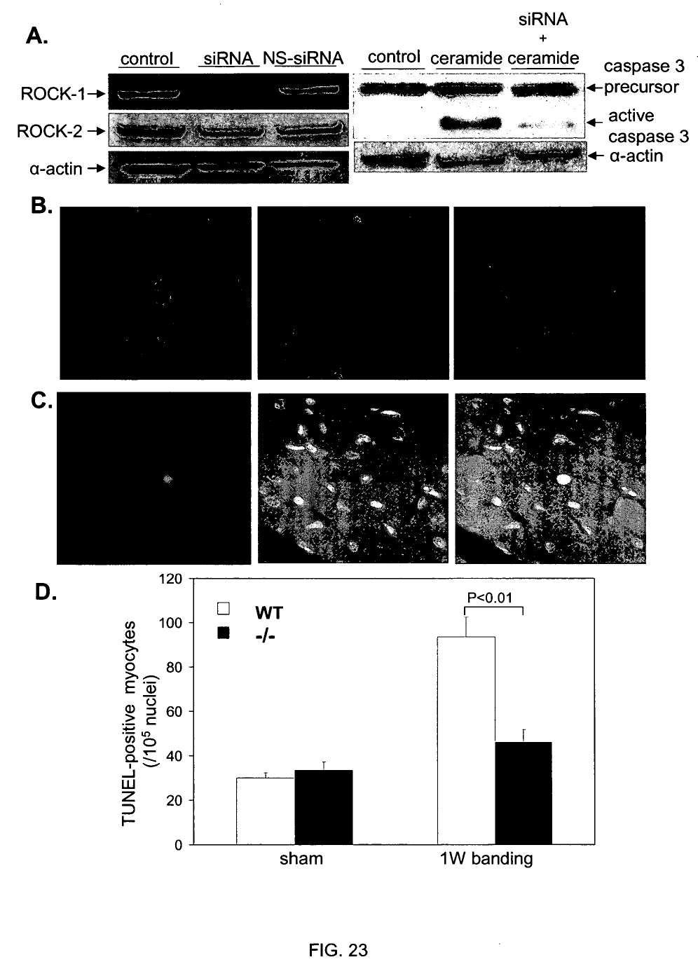

Since overexpression of ROCKΔ1 is sufficient to initiate a caspase cascade, in a specific embodiment of the invention blocking expression in ROCK-1 may prevent cardiomyocytes from apoptosis induced by apoptotic stimuli. As indicated in FIG. 23A, a specific siRNA significantly knocked down ROCK-1 expression without interrupting expression of the other Rho kinase isoform, ROCK-2, in cardiomyocytes (top left panel). Application of this siRNA also inhibited caspase 3 activation induced by ceramide (top right panel). Without siRNA treatment, ceramide strongly induced caspase 3 activation, along with cellular apoptosis. Fluorescent staining showed that pre-treatment with siRNA protected cardiomyocytes against ceramide-induced apoptosis with almost intact myofilament structure compared to a disorganized and damaged cellular structure in non-siRNA treated cells (FIG. 23B).

In order to further characterize the role of ROCK-1 in this proteolytic cascade process in vivo, ROCK-1 null mice were generated. ROCK-1^{-/-} mice were viable, which allowed us to subject these mice to pathophysiological conditions, such as pressure overload. Consistent with the siRNA results, it was found that ROCK-1^{-/-} mice exhibited significantly reduced myocyte apoptosis as compared to WT mice when induced by pressure overload through aortic banding (FIGS. 22C and 22D). Therefore, blocked ROCK-1 expression attenuated caspase 3 activity and contributed to myocardial protection.

### Example 15

### Significance of the Present Invention

The inventors conducted highly selective blockades of ROCK-1 expression in cardiomyocyte culture by application of siRNA as well as loss-of-function studies in the genetically modified mouse heart. The data support an in vivo role for caspase 3-mediated ROCK-1 cleavage and activation in facilitating myocyte apoptosis in heart failure. The pro-apoptotic effect of Rho kinase has also been suggested by other studies (Lai et al., 2002; Lai et al., 2003; Petrache et al., 2003). In an isolated perfusion rat heart study, pharmacologic inhibition of Rho kinase significantly reduced the level of myocyte apoptosis induced by ischemia/reperfusion (Bao et al., 2004).

The inventors addressed the molecular mechanism for Rho kinase's pro-apoptotic effect and demonstrated that the constitutively active ROCK-1 mutant generated by cleavage (Coleman et al., 2001; Sebbagh et al., 2001) directly activated caspase 3 and led to myocyte apoptosis. This proteolytic activation was associated with the inhibition of AKT activity via the increase in PTEN activation. PTEN has been demonstrated to act as a negative regulator of AKT in opposition to the PIP3/AKT signaling pathway (Oudit et al., 2004; Goberdhan et al., 1999; Crackower et al., 2002; Schwartzbauer and Robbins, 2001). AKT has been recognized as an anti-apoptotic factor (Franke et al., 1997; Latronico et al., 2004). It has been reported that PTEN can be activated by active RhoA and phosphorylated by ROCK-1 in vitro (Li et al., 2005; Meili et al., 2005). nhibition of Rho kinase leads to activation of AKT and cardiovascular protection (Wolfrum et al., 2004). To further this observation, the inventors demonstrated that active ROCK-1 directly increased PTEN activity and subsequently decreased phosphorylation of AKT. Blocking expression of PTEN reverted AKT to control levels even with the active ROCK-1 treatment, indicating that the pro-apoptotic effect of activated ROCK-1 was associated with the activation of PTEN and the dephosphorylation of AKT. Cleaved ROCK-1 in failing hearts is one of the mechanisms that may directly contribute to myocyte apoptosis.

Conclusion

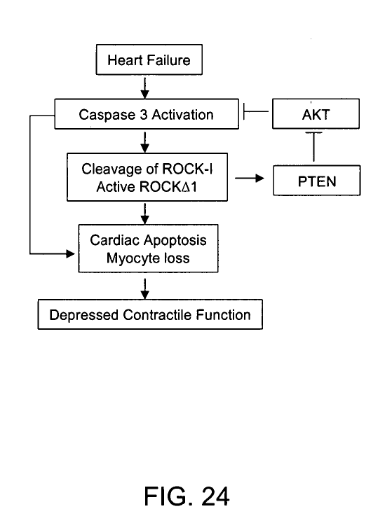

In summary (FIG. 24), the inventors demonstrated that ROCK-1 is one of the targets for activated caspase 3 in human failing hearts and transgenic mouse hearts with severe cardiomyopathy in the absence of large-scale apoptosis. The cleavage resulted in an active ROCK-1 kinase, which further induced caspase 3 activation and myocyte apoptosis and generated a positive feed-forward loop for caspase cascade activation. This pro-apoptotic effect is associated with the activation of PTEN and the dephosphorylation of AKT. Deficiency of ROCK-1 significantly reduced cardiac apoptosis induced by ceramide or pressure overload. Therapeutic inhibition of ROCK-1 may be a useful alternative for treatment of severe heart failure.

### Example 16

### Searching for PH Domain Inhibition Using the Luminescent Kinase Assay

An exemplary screen is described for identifying agents that selectively inhibit ROCK 1. p160 ROCK comprises three domains: a kinase domain, coiled-coil domain and pleckstrin homology (PH) domain. PH domain can bind to the kinase domain and inhibit the kinase activity. Cleavage of the PH domain region of ROCK leads to the constitutively activated kinase during the apotosis. Therefore, the inventors focus on the interaction between PH domain and kinase domain, and in a specific embodiment of the assay assume that free PH domain is acting as the inhibitor to kinase domain.

The key region of the PH domain, which is most critical for the inhibition, is searched. From that region, the inventors develop a peptide-based compound to inhibit the activity of kinase domain. Study of a small compound library often leads to the identity of an ATP-competitive inhibitor. In a specific embodiment, the approach of the inventors is different from current investigations of kinase inhibitors and results in identification of the ATP-noncompetitive inhibitor.

In a particular embodiment, the Kinase-Glo luminescent kinase assay from Promega (Madison, Wis.) is employed, which comprises a method of measuring kinase activity by quantifying the amount of ATP remaining in solution following a kinase reaction. The inventors demonstrate that free PH domain has inhibition activity against myeline basic protein (MBP) kinase (MBP-kinase), similar as what is seen with Y-27632. In additional embodiments, the Kₘ and/or k_{cat} for SRF-N against MBP-Kinase and the IC₅₀ or Kᵢ for PH domain are measured, such as by standard methods in the art.

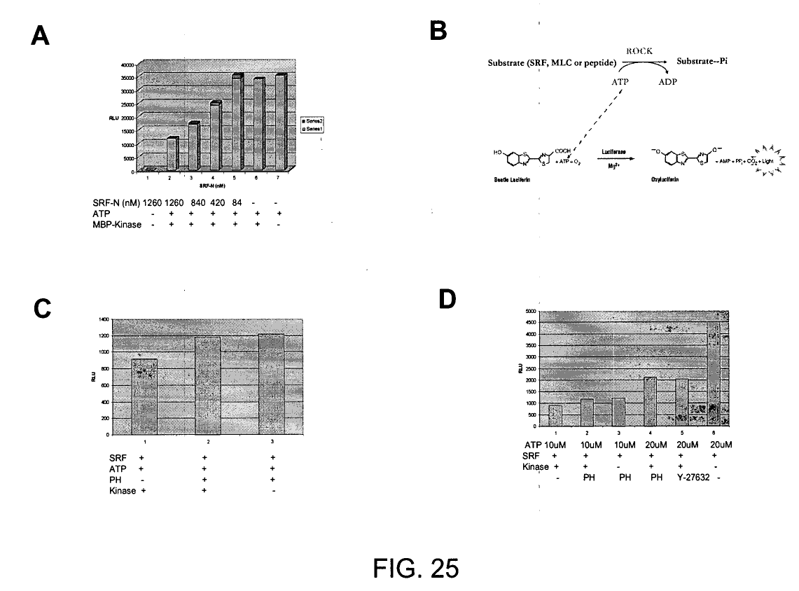

FIG. 25B illustrates the role of ROCK in processing of its substrates in the presence of ATP. FIG. 25A demonstrates performance of the Kinase-Glo luminescent kinase assay using SRF-N (the 32 kDa fragment or SRF following cleavage) as substrate in the presence or absence of MBP-kinase and ATP, and the luminescence is measured in relative light units (RLU). FIGS. 25C and 25D demonstrate PH domain inhibition.

### Example 17

### Exemplary Materials and Methods

Exemplary materials and methods are provided herein.

Human failing heart tissues. Myocardial samples were obtained from thirteen patients with end-stage heart failure at the time of transplant: seven patients had ischemic cardiomyopathy (ICM), five had dilated cardiomyopathy (DCM), and one had hypertrophic cardiomyopathy (HCM). An additional group of ten patients, including eight with DCM and two with ICM, had been maintained on LVAD until transplant. Samples were obtained from seven patients who died of non-cardiac causes for use as a control group. The left ventricular ejection fraction was less than 20% in all heart failure patients.

Cell culture, plasmid constructs, and recombinant adenoviruses. Neonatal rat cardiomyocytes were isolated and cultured in DMEM/F12 medium (1:1) with 10% horse serum (DF10); cells were ready for transfection or virus infection 40 hours after plating. For immunostaining, cells were cultured on pre-coated (with 0.2% gelatin) coverslips. Transfections were performed using LipofectAMINE 2000 (Invitrogen) with Opti-MEM I Reduced Serum Medium (Invitrogen). After 6 hr or O/N, cells were cultured in DF10. With human embryonic kidney (HEK) A293T cells, cells were cultured in DMEM with 10% fetal bovine serum. The cDNA for full length human ROCK-1 cloned into the pCAG-myc vector (pCAG-ROCK-1) was kindly provided by Dr. Narumiya. All ROCK-1 and mutant constructs were tagged with a Myc epitope at the amino-terminus. The Asp718-MscI cDNA fragment encoding ROCKΔ1 mutant (residues 1-1080) was also cloned into the pCAG-myc vector (pCAG-ROCKΔ1) (Ishizaki et al., 1997). A signal lysine at residue 105 was replaced by alanine to generate a kinase deficient mutant, pCAG-ROCK-1KD. The adenoviral vector, Ad-G/iCasp 3, expressing conditional caspase 3, was generously provided by Drs. David Spencer and Kevin Slawin at Baylor College of Medicine. Addition of CID, AP20187 (50 nM, 24 hr), provoked the aggregation and activation of caspase 3 as well as rapid apoptosis (Shariat et al., 2001; Mallet et al., 2002).

Apoptosis assay and immunofluorescence analysis. Apoptosis was evaluated using caspase 3 activity and poly(ADP-ribose) polymerase (PARP) cleavage with Western blot in human and mouse heart tissues. With regard to apoptotic cultured neonatal cardiomyocytes, a caspase 3 sensor (BD Biosciences) was introduced to detect the onset of caspase 3 activity (FIG. 9). The vector encodes EYFP (enhanced yellow fluorescent protein) fused with NES (nuclear export signal) and NLS (nuclear localization signal). A caspase 3 specific cleavage site is located between EYFP and NES. When caspase 3 is inactive, the dominant NES directs EYFP to the cytosol. Upon induction of apoptosis, the export signal is removed by active caspase 3, which triggers the redistribution of EYFP from the cytosol to the nucleus via NLS. The quantification was performed by counting the number of transfected cells with the nuclear localization of fluorescent fusion protein over the total transfected cells. Alexa Fluor 594 phalloidin staining (Molecular Probes) and DAPI staining were applied for F-actin and cellular nuclei visualization, respectively.

Animal models with different apoptotic levels. Three transgenic mouse lines with different apoptotic levels were used. Epitope-tagged HGK (hepatocyte progenitor kinase-like/germinal center kinase-like kinase) was overexpressed in mouse myocardium using the αMHC promoter (Xie et al., 2004; Subramaniam et al., 1991). αMHC-Gq mice were kindly provided by G. Dom at University of Cincinnati (D'Angelo et al., 1997; Adams et al., 1998; Sakata et al., 1998). Bi-transgenic mice overexpressing αMHC-Gq-HGK mice were generated by breeding HGK with Gq mice (Xie et al., 2004). All experiments were performed in ten-week old mice with an isogenic FVB/N background. No early lethality resulted from cardiac overexpression of exogenous HGK and Gq alone.

siRNAs application for ROCK-1 and PTEN knockdown and ceramide-induced apoptosis. To knockdown ROCK-1 expression in neonatal rat cardiomyocytes, 100 nM siRNA (Ambion) was used with co-transfection of caspase 3 sensor vector as described above. Same amount of siRNA specific for PTEN (Ambion) was applied to human HEK cells. Cells were treated with ceramide 40 hr after the transfection at 50 μg/ml for 2 hr to induce apoptosis.

ROCK-1 knockout mice. Generation and characterization of ROCK-1^{-/-} mice have been described previously (Bo et al., 2004).

Transverse aortic banding. Transverse aortic banding was conducted in 12-week old adult wild type (WT) and ROCK-1^{-/-} mice (Hartley et al., 2002). Briefly, both ROCK-1^{-/-} and WT mice received a comparable load, based on the right-to-left carotid artery flow velocity ratio (from 5:1 to 10:1) after constricting the transverse aorta. As a control, a sham operation without occlusion was performed on respective age-matched mice.

Western blot and Malachite green assay. Anti-ROCK-1, caspase 3, PARP, His, α-actin antibodies (Santa Cruz), anti-phospho-AKTser473 (Cell Signaling) and anti-RTEN (Upstate) were purchased. Protein samples for Western blot were prepared and separated as described earlier (Chang et al., 2000). Even loadings were confirmed by Ponceau staining and antibody probed for actin. PTEN activity was evaluated by Malachite green assay (Upstate). 500 μg of cell lysate protein was used for immunoprecipitation.

Statistical analysis. Data were analyzed by one-way ANOVA followed by Bonferroni t-test. (SigmaStat, SPSS Inc.). A P<0.05 was considered significant. Data are presented as mean±SEM.

### REFERENCES

All patents and publications mentioned in the specification are indicative of the levels of those skilled in the art to which the invention pertains. All patents and publications are herein incorporated by reference to the same extent as if each individual publication was specifically and individually indicated to be incorporated by reference.

### PUBLICATIONS

Adams, J. W., Sakata, Y., Davis, M. G., Sah, V. P., Wang, Y., Liggett, S. B., Chien, K. R., Brown, J. H., and Dom, G. W., 2nd (1998) Enhanced Galphaq signaling: a common pathway mediates cardiac hypertrophy and apoptotic heart failure. Proc Natl Acad Sci USA 95, 10140-10145.

Agah, R., Frenkel, P. A., French, B. A., Michael, L. H., Overbeek, P. A., and Schneider, M. D. (1997) Gene recombination in postmitotic cells. Targeted expression of Cre recombinase provokes cardiac-restricted, site-specific rearrangement in adult ventricular muscle in vivo. J Clin Invest 100, 169-179.

Aikawa, R., Komuro, I., Yamazaki, T., Zou, Y., Kudoh, S., Zhu, W., Kadowaki, T., and Yazaki, Y. (1999) Rho family small G proteins play critical roles in mechanical stress-induced hypertrophic responses in cardiac myocytes. Circ Res 84, 458-466.

Aklilu, M., et al., Phase II study of flavopiridol in patients with advanced colorectal cancer. Ann Oncol, 2003. 14(8): p. 1270-3.

Amano, M., Chihara, K., Kimura, K., Fukata, Y., Nakamura, N., Matsuura, Y., and Kaibuchi, K. (1997) Formation of actin stress fibers and focal adhesions enhanced by Rho-kinase. Science 275, 1308-1311.

Amano, M., Fukata, Y., and Kaibuchi, K. (2000) Regulation and functions of Rho-associated kinase. Exp Cell Res 261, 44-51.

Anantharam, V., Kitazawa, M., Wagner, J., Kaul, S., and Kanthasamy, A. G. (2002) Caspase-3-dependent proteolytic cleavage of protein kinase Cdelta is essential for oxidative stress-mediated dopaminergic cell death after exposure to methylcyclopentadienyl manganese tricarbonyl. J Neurosci 22, 1738-1751.

Aoki, H., Izumo, S., and Sadoshima, J. (1998) Angiotensin II activates RhoA in cardiac myocytes: a critical role of RhoA in angiotensin II-induced premyofibril formation. Circ Res 82, 666-676.

Arai, M., Matsui, H., and Periasamy, M. (1994) Sarcoplasmic reticulum gene expression in cardiac hypertrophy and heart failure. Circ Res 74, 555-564.

Arber, S., Barbayannis, F. A., Hanser, H., Schneider, C., Stanyon, C. A., Bernard, O., and Caroni, P. (1998) Regulation of actin dynamics through phosphorylation of cofilin by LIM-kinase. Nature 393, 805-809.

Aronow, B. J., Toyokawa, T., Canning, A., Haghighi, K., Delling, U., Kranias, E., Molkentin, J. D., and Dorn, G. W., 2nd (2001) Divergent transcriptional responses to independent genetic causes of cardiac hypertrophy. Physiol Genomics 6, 19-28.

Astrom, A. K., et al., Conserved mechanism of PLAG1 activation in salivary gland tumors with and without chromosome 8q12 abnormalities: identification of SII as a new fusion partner gene. Cancer Res, 1999. 59(4): p. 918-23.

Bains, M. A., I. Giles, and D. H. Wright, Distribution and configuration of c-myc RNA during transcriptional attenuation in differentiating cells in-situ. Histochem Cell Biol, 1997. 107(3): p. 259-63.

Barrans, J. D., Allen, P. D., Stamatiou, D., Dzau, V. J., and Liew, C. C. (2002) Global gene expression profiling of end-stage dilated cardiomyopathy using a human cardiovascular-based cDNA microarray. Am J Pathol 160, 2035-2043.

Beguin, P., Nagashima, K., Gonoi, T., Shibasaki, T., Takahashi, K., Kashima, Y., Ozaki, N., Geering, K., Iwanaga, T., and Seino, S. (2001) Regulation of Ca2+ channel expression at the cell surface by the small G-protein kir/Gem. Nature 411, 701-706.

Begum, N., Sandu, O. A., Ito, M., Lohmann, S. M., and Smolenski, A. (2002) Active Rho kinase (ROK-alpha ) associates with insulin receptor substrate-1 and inhibits insulin signaling in vascular smooth muscle cells. J Biol Chem 277, 6214-6222.

Belaguli, N. S., Zhou, W., Trinh, T. H., Majesky, M. W., and Schwartz, R. J. (1999) Dominant negative murine serum response factor: alternative splicing within the activation domain inhibits transactivation of serum response factor binding targets. Mol Cell Biol 19, 4582-4591.

Belotserkovskaya, R., et al., Transcription through chromatin: understanding a complex FACT. Biochem Biophys Acta, 2004. 1677(1-3): p. 87-99.

Benson, R. H., et al., DNA-dependent RNA polymerase II stimulatory factors from calf thymus: purification and structural studies. Biochemistry, 1978. 17(8): p. 1387-96.

Bernard, D., et al., Myc confers androgen-independent prostate cancer cell growth. J Clin Invest, 2003. 112(11): p. 1724-31.

Bertolotto, C., Ricci, J. E., Luciano, F., Mari, B., Chambard, J. C., and Auberger, P. (2000) Cleavage of the serum response factor during death receptor-induced apoptosis results in an inhibition of the c-FOS promoter transcriptional activity. J Biol Chem 275, 12941-12947.

Blancato, J., et al., Correlation of amplification and overexpression of the c-myc oncogene in high-grade breast cancer: FISH, in situ hybridisation and immunohistochemical analyses. Br J Cancer, 2004. 90(8): p. 1612-9.

Blankenberg, F., Narula, J., and Strauss, H. W. (1999) In vivo detection of apoptotic cell death: a necessary measurement for evaluating therapy for myocarditis, ischemia, and heart failure. J Nucl Cardiol 6, 531-539.

Bueno, O. F., and Molkentin, J. D. (2002) Involvement of extracellular signal-regulated kinases ½ in cardiac hypertrophy and cell death. Circ Res 91, 776-781.

Bueno, O. F., De Windt, L. J., Tymitz, K. M., Witt, S. A., Kimball, T. R., Klevitsky, R., Hewett, T. E., Jones, S. P., Lefer, D. J., Peng, C. F., Kitsis, R. N., and Molkentin, J. D. (2000) The MEK1-ERK1/2 signaling pathway promotes compensated cardiac hypertrophy in transgenic mice. Embo J 19, 6341-6350.

Burcin, M. M., Schiedner, G., Kochanek, S., Tsai, S. Y., and O'Malley, B. W. (1999) Adenovirus-mediated regulable target gene expression in vivo. Proc Natl Acad Sci USA 96, 355-360.

Chang, J., Wei, L., Otani, T., Youker, K. A., Entman, M. E., and Schwartz, R. J. (2002) Inhibitory serum response factors caused by caspase 3 cleavage accumulated in human failing hearts and was reversed by the left ventricular assist device. Circulation 106, 11109.

Charron, F., Tsimiklis, G., Arcand, M., Robitaille, L., Liang, Q., Molkentin, J. D., Meloche, S., and Nemer, M. (2001) Tissue-specific GATA factors are transcriptional effectors of the small GTPase RhoA. Genes Dev 15, 2702-2719.

Chen, L., Hahn, H., Wu, G., Chen, C. H., Liron, T., Schechtman, D., Cavallaro, G., Banci, L., Guo, Y., Bolli, R., Dorn, G. W., 2nd, and Mochly-Rosen, D. (2001) Opposing cardioprotective actions and parallel hypertrophic effects of delta PKC and epsilon PKC. Proc Natl Acad Sci USA 98, 11114-11119.

Chen, X.Q. et al. Characteriztion of RhoA-binding kinase ROCka implication of the pleckstrin homology domain in ROKa function using region-specifi antiobiodies. JBC, 2002, 277,12680-12688.

Chi, X., Zhang, S. X., Yu, W., DeMayo, F. J., Rosenberg, S. M., and Schwartz, R. J. (2003) Expression of Nkx2-5-GFP bacterial artificial chromosome transgenic mice closely resembles endogenous Nkx2-5 gene activity. Genesis 35, 220-226.

Chien, K. R. (1999) Stress pathways and heart failure. Cell 98, 555-558.

Chrzan, P., et al., Amplification of c-myc gene and overexpression of c-Myc protein in breast cancer and adjacent non-neoplastic tissue. Clin Biochem, 2001. 34(7): p. 557-62.

Coleman, M. L., Sahai, E. A., Yeo, M., Bosch, M., Dewar, A., and Olson, M. F. (2001) Membrane blebbing during apoptosis results from caspase-mediated activation of ROCK I. Nat Cell Biol 3, 339-345.

Communal, C., Sumandea, M., de Tombe, P., Narula, J., Solaro, R. J., and Hajjar, R. J. (2002) Functional consequences of caspase activation in cardiac myocytes. Proc Natl Acad Sci USA 99, 6252-6256.

Condorelli, G., Roncarati, R., Ross, J., Jr., Pisani, A., Stassi, G., Todaro, M., Trocha, S., Drusco, A., Gu, Y., Russo, M. A., Frati, G., Jones, S. P., Lefer, D. J., Napoli, C., and Croce, C. M. (2001) Heart-targeted overexpression of caspase3 in mice increases infarct size and depresses cardiac function. Proc Natl Acad Sci USA 98, 9977-9982.

Dahmus, M. E., Phosphorylation of eukaryotic DNA-dependent RNA polymerase. Identification of calf thymus RNA polymerase subunits phosphorylated by two purified protein kinases, correlation with in vivo sites of phosphorylation in HeLa cell RNA polymerase II. J Biol Chem, 1981. 256(7): p. 3332-9.

Dorn, G. W., 2nd (2002) Adrenergic pathways and left ventricular remodeling. J Card Fail 8, S370-373.

Dorn, G. W., 2nd, Robbins, J., and Sugden, P. H. (2003) Phenotyping hypertrophy: eschew obfuscation. Circ Res 92, 1171-1175.

Drewett, V., Devitt, A., Saxton, J., Portman, N., Greaney, P., Cheong, N. E., Alnemri, T. F., Alnenri, E., and Shaw, P. E. (2001) Serum response factor cleavage by caspases 3 and 7 linked to apoptosis in human BJAB cells. J Biol Chem 276, 33444-33451.

Ellwood-Yen, K., et al., Myc-driven murine prostate cancer shares molecular features with human prostate tumors. Cancer Cell, 2003. 4(3): p. 223-38.

Elsasser, A., Suzuki, K., and Schaper, J. (2000) Unresolved issues regarding the role of apoptosis in the pathogenesis of ischemic injury and heart failure. J Mol Cell Cardiol 32, 711-724.

Emery, J. G., E. H. Ohlstein, and M. Jaye, Therapeutic modulation of transcription factor activity. Trends Pharmacol Sci, 2001. 22(5): p. 233-40.

Farah, S., Agazie, Y., Ohan, N., Ngsee, J. K., and Liu, X. J. (1998) A rho-associated protein kinase, ROKalpha, binds insulin receptor substrate-1 and modulates insulin signaling. J Biol Chem 273, 4740-4746.

Farley, F. W., Soriano, P., Steffen, L. S., and Dymecki, S. M. (2000) Widespread recombinase expression using FLPeR (flipper) mice. Genesis 28, 106-110.

Fluiter, K., et al., In vivo tumor growth inhibition and biodistribution studies of locked nucleic acid (LNA) antisense oligonucleotides. Nucleic Acids Res, 2003.31(3): p. 953-62.

Frankel, A. D., and Pabo, C. 0. (1988) Cellular uptake of the tat protein from human immunodeficiency virus. Cell 55, 1189-1193.

Fujio, Y., Nguyen, T., Wencker, D., Kitsis, R. N., and Walsh, K. (2000) Akt promotes survival of cardiomyocytes in vitro and protects against ischemia-reperfusion injury in mouse heart. Circulation 101, 660-667.

Garcia-Martinez, L. F., et al., In vitro high-throughput screening assay for modulators of transcription. Anal Biochem, 2002.301(1): p. 103-10.

Gartel, A. L., and Tyner, A. L. (2002) The role of the cyclin-dependent kinase inhibitor p21 in apoptosis. Mol Cancer Ther 1, 639-649.

Gaussin, V., Van de Putte, T., Mishina, Y., Hanks, M. C., Zwijsen, A., Huylebroeck, D., Behringer, R. R., and Schneider, M. D. (2002) Endocardial cushion and myocardial defects after cardiac myocyte-specific conditional deletion of the bone morphogenetic protein receptor ALK3. Proc Natl Acad Sci USA 99, 2878-2883.

Gelmann, E. P. and O. J. Semmes, Expression of genes and proteins specific for prostate cancer. J Urol, 2004. 172(5 Pt 2): p. 523-6; discussion S26-7.

Gnatt, A., J. Fu, and R. D. Komberg, Formation and crystallization of yeast RNA polymerase II elongation complexes. J Biol Chem, 1997. 272(49): p. 30799-805.

Grad, J. M., et al., Multiple androgen response elements and a Myc consensus site in the androgen receptor (AR) coding region are involved in androgen-mediated up-regulation of AR messenger RNA. Mol Endocrinol, 1999. 13(11): p. 1896-911.

Green, M., and Loewenstein, P. M. (1988) Autonomous functional domains of chemically synthesized human immunodeficiency virus tat trans-activator protein. Cell 55, 1179-1188.

Hall, A. (1994) Small GTP-binding proteins and the regulation of the actin cytoskeleton. Annu Rev Cell Biol 10, 31-54.

Hardt, S. E., and Sadoshima, J. (2002) Glycogen synthase kinase-3beta: a novel regulator of cardiac hypertrophy and development. Circ Res 90, 1055-1063.

Hauck, L., Hansmann, G., Dietz, R., and von Harsdorf, R. (2002) Inhibition of hypoxia-induced apoptosis by modulation of retinoblastoma protein-dependent signaling in cardiomyocytes. Circ Res 91, 782-789.

Higashi, M., Shimokawa, H., Hattori, T., Hiroki, J., Mukai, Y., Morikawa, K., Ichiki, T., Takahashi, S., and Takeshita, A. (2003) Long-term inhibition of Rho-kinase suppresses angiotensin II-induced cardiovascular hypertrophy in rats in vivo: effect on endothelial NAD(P)H oxidase system. Circ Res 93, 767-775.

Hirota, H., Chen, J., Betz, U. A., Rajewsky, K., Gu, Y., Ross, J., Jr., Muller, W., and Chien, K. R. (1999) Loss of a gp130 cardiac muscle cell survival pathway is a critical event in the onset of heart failure during biomechanical stress. Cell 97, 189-198.

Hodo, H. G., 3rd and S. P. Blatti, Purification using polyethylenimine precipitation and low molecular weight subunit analyses of calf thymus and wheat germ DNA-dependent RNA polymerase II. Biochemistry, 1977. 16(11): p. 2334-43.

hor, J., E. Ben-Asher, and Y. Aloni, Transcription elongation of the murine omithine decarboxylase (ODC) gene is regulated in vitro at two downstream elements by different attenuation mechanisms. Oncogene, 1995. 10(8): p. 1587-96.

Hoshijima, M., and Chien, K. R. (2002) Mixed signals in heart failure: cancer rules. J Clin Invest 109, 849-855.

Hoshijima, M., Sah, V. P., Wang, Y., Chien, K. R., and Brown, J. H. (1998) The low molecular weight GTPase Rho regulates myofibril formation and organization in neonatal rat ventricular myocytes. Involvement of Rho kinase. J Biol Chem 273, 7725-7730.

Hwang, J. J., Allen, P. D., Tseng, G. C., Lam, C. W., Fananapazir, L., Dzau, V. J., and Liew, C. C. (2002) Microarray gene expression profiles in dilated and hypertrophic cardiomyopathic end-stage heart failure. Physiol Genomics 10, 31-44.

Ikeda, H., Nagashima, K., Yanase, M., Tomiya, T., Arai, M., Inoue, Y., Tejima, K., Nishikawa, T., Omata, M., Kimura, S., and Fujiwara, K. (2003) Involvement of Rho/Rho kinase pathway in regulation of apoptosis in rat hepatic stellate cells. Am J Physiol Gastrointest Liver Physiol 285, G880-886.

Ishizaki, T., Maekawa, M., Fujisawa, K., Okawa, K., Iwamatsu, A., Fujita, A., Watanabe, N., Saito, Y., Kakizuka, A., Morii, N., and Narumiya, S. (1996) The small GTP-binding protein Rho binds to and activates a 160 kDa Ser/Thr protein kinase homologous to myotonic dystrophy kinase. Embo J 15, 1885-1893.

Ishizaki, T., Uehata, M., Tamechika, I., Keel, J., Nonomura, K., Maekawa, M., and Narumiya, S. (2000) Pharmacological properties of Y-27632, a specific inhibitor of rho-associated kinases. Mol Pharmacol 57, 976-983.

Itoh, K., Yoshioka, K., Akedo, H., Uehata, M., Ishizaki, T., and Narumiya, S. (1999) An essential part for Rho-associated kinase in the transcellular invasion of tumor cells. Nat Med 5, 221-225.

Iversen, P. L., et al., Efficacy of antisense morpholino oligomer targeted to c-myc in prostate cancer xenograft murine model and a Phase I safety study in humans. Clin Cancer Res, 2003. 9(7): p. 2510-9.

Jacoby, J. J., Kalinowski, A., Liu, M. G., Zhang, S. S., Gao, Q., Chai, G. X., Ji, L., Iwamoto, Y., Li, E., Schneider, M., Russell, K. S., and Fu, X. Y. (2003) Cardiomyocyte-restricted knockout of STAT3 results in higher sensitivity to inflammation, cardiac fibrosis, and heart failure with advanced age. Proc Natl Acad Sci USA 100, 12929-12934.

Jain, M., et al., Sustained loss of a neoplastic phenotype by brief inactivation of MYC. Science, 2002. 297(5578): p. 102-4.

Jenkins, R. B., et al., Detection of c-myc oncogene amplification and chromosomal anomalies in metastatic prostatic carcinoma by fluorescence in situ hybridization. Cancer Res, 1997. 57(3): p. 524-31.

Kang, P. M., and Izumo, S. (2000) Apoptosis and heart failure: A critical review of the literature. Circ Res 86, 1107-1113.

Kaul, S., Kanthasamy, A., Kitazawa, M., Anantharam, V., and Kanthasamy, A. G. (2003) Caspase-3 dependent proteolytic activation of protein kinase C delta mediates and regulates 1-methyl-4-phenylpyridinium (MPP+)-induced apoptotic cell death in dopaminergic cells: relevance to oxidative stress in dopaminergic degeneration. Eur J Neurosci 18, 1387-1401.

Keene, R. G., et al., Transcriptional pause, arrest and termination sites for RNA polymerase II in mammalian N— and c-myc genes. Nucleic Acids Res, 1999. 27(15): p. 3173-82.

Kelly, W. K., O. A. O'Connor, and P. A. Marks, Histone deacetylase inhibitors: from target to clinical trials. Expert Opin Investig Drugs, 2002. 11(12): p. 1695-713.

Kerppola, T. K. and C. M. Kane, Analysis of the signals for transcription termination by purified RNA polymerase II. Biochemistry, 1990. 29(1): p. 269-78.

Kerppola, T. K. and C. M. Kane, Intrinsic sites of transcription termination and pausing in the c-myc gene. Mol Cell Biol, 1988. 8(10): p. 4389-94.

Khorasanizadeh, S., The nucleosome: from genomic organization to genomic regulation. Cell, 2004. 116(2): p. 259-72.

Kimura, K., Ito, M., Amano, M., Chihara, K., Fukata, Y., Nakafuku, M., Yamamori, B., Feng, J., Nakano, T., Okawa, K., Iwamatsu, A., and Kaibuchi, K. (1996) Regulation of myosin phosphatase by Rho and Rho-associated kinase (Rho-kinase). Science 273, 245-248.

Kobayashi, N., Horinaka, S., Mita, S., Nakano, S., Honda, T., Yoshida, K., Kobayashi, T., and Matsuoka, H. (2002) Critical role of Rho-kinase pathway for cardiac performance and remodeling in failing rat hearts. Cardiovasc Res 55, 757-767.

Kuwahara, K., Saito, Y., Nakagawa, O., Kishimoto, I., Harada, M., Ogawa, E., Miyamoto, Y., Hamanaka, I., Kajiyama, N., Takahashi, N., Izumi, T., Kawakami, R., Tamura, N., Ogawa, Y., and Nakao, K. (1999) The effects of the selective ROCK inhibitor, Y27632, on ET-1-induced hypertrophic response in neonatal rat cardiac myocytes-possible involvement of Rho/ROCK pathway in cardiac muscle cell hypertrophy. FEBS Lett 452, 314-318.

Lai, J. M., Hsieh, C. L., and Chang, Z. F. (2003) Caspase activation during phorbol ester-induced apoptosis requires ROCK-dependent myosin-mediated contraction. J Cell Sci 116, 3491-3501.

Lai, J. M., Wu, S., Huang, D. Y., and Chang, Z. F. (2002) Cytosolic retention of phosphorylated extracellular signal-regulated kinase and a Rho-associated kinase-mediated signal impair expression of p21(Cipl/Wafl) in phorbol 12-myristate-13-acetate-induced apoptotic cells. Mol Cell Biol 22, 7581-7592.

Lakso, M., Pichel, J. G., Gorman, J. R., Sauer, B., Okamoto, Y., Lee, E., Alt, F. W., and Westphal, H. (1996) Efficient in vivo manipulation of mouse genomic sequences at the zygote stage. Proc Natl Acad Sci USA 93, 5860-5865.

Latil, A., et al., htert expression correlates with MYC over-expression in human prostate cancer. Int J Cancer, 2000. 89(2): p. 172-6.

Laufs, U., Kilter, H., Konkol, C., Wassmann, S., Bohm, M., and Nickenig, G. (2002) Impact of HMG CoA reductase inhibition on small GTPases in the heart. Cardiovasc Res 53, 911-920.

Laugwitz, K. L., Moretti, A., Weig, H. J., Gillitzer, A., Pinkemell, K., Ott, T., Pragst, I., Stadele, C., Seyfarth, M., Schomig, A., and Ungerer, M. (2001) Blocking caspase-activated apoptosis improves contractility in failing myocardium. Hum Gene Ther 12, 2051-2063.

Lee, K. K., Ohyama, T., Yajima, N., Tsubuki, S., and Yonehara, S. (2001) MST, a physiological caspase substrate, highly sensitizes apoptosis both upstream and downstream of caspase activation. J Biol Chem 276, 19276-19285.

Lee, T. C., and Schwartz, R. J. (1992) Using proteases to avoid false identification of DNA-protein complexes in gel shift assays. Biotechniques 12, 486-490.

Lei, K., Nimnual, A., Zong, W. X., Kennedy, N. J., Flavell, R. A., Thompson, C. B., Bar-Sagi, D., and Davis, R. J. (2002) The Bax subfamily of Bcl2-related proteins is essential for apoptotic signal transduction by c-Jun NH(2)-terminal kinase. Mol Cell Biol 22, 4929-4942.

Leone, G., et al., Inhibitors of DNA methylation in the treatment of hematological malignancies and MDS. Clin hmmunol, 2003. 109(1): p. 89-102.

Leung, T., Chen, X. Q., Manser, E., and Lim, L. (1996) The pI60 RhoA-binding kinase ROK alpha is a member of a kinase family and is involved in the reorganization of the cytoskeleton. Mol Cell Biol 16, 5313-5327.

Li, L., Liu, Z., Mercer, B., Overbeek, P., and Olson, E. N. (1997) Evidence for serum response factor-mediated regulatory networks governing SM22alpha transcription in smooth, skeletal, and cardiac muscle cells. Dev Biol 187, 311-321.

Li, X., Liu, L., Tupper, J. C., Bannerman, D. D., Winn, R. K., Sebti, S. M., Hamilton, A. D., and Harlan, J. M. (2002) Inhibition of protein geranylgeranylation and RhoA/RhoA kinase pathway induces apoptosis in human endothelial cells. J Biol Chem 277, 15309-15316.

Liang, Q., Bueno, O. F., Wilkins, B. J., Kuan, C. Y., Xia, Y., and Molkentin, J. D. (2003) c-Jun N-terminal kinases (JNK) antagonize cardiac growth through cross-talk with calcineurin-NFAT signaling. Embo J 22, 5079-5089.

Lu, C., et al., cFos is critical for MCF-7 breast cancer cell growth. Oncogene, 2005.

Luo, Y., et al., Transcription factor Fos-related antigen 1 is an effective target for a breast cancer vaccine. Proc Natl Acad Sci USA, 2003. 100(15): p. 8850-5.

Maekawa, M., Ishizaki, T., Boku, S., Watanabe, N., Fujita, A., Iwamatsu, A., Obinata, T., Ohashi, K., Mizuno, K., and Narumiya, S. (1999) Signaling from Rho to the actin cytoskeleton through protein kinases ROCK and LIM-kinase. Science 285, 895-898.

Mallet, V. O., Mitchell, C., Guidotti, J. E., Jaffray, P., Fabre, M., Spencer, D., Arnoult, D., Kahn, A., and Gilgenkrantz, H. (2002) Conditional cell ablation by tight control of caspase-3 dimerization in transgenic mice. Nat Biotechnol 20, 1234-1239.

Mann, D. L. (2003) Stress-activated cytokines and the heart: from adaptation to maladaptation. Annu Rev Physiol 65, 81-101.

Matsui, T., Amano, M., Yamamoto, T., Chihara, K., Nakafuku, M., Ito, M., Nakano, T., Okawa, K., Iwamatsu, A., and Kaibuchi, K. (1996) Rho-associated kinase, a novel serine/threonine kinase, as a putative target for small GTP binding protein Rho. Embo J 15, 2208-2216.

Matsui, T., Nagoshi, T., and Rosenzweig, A. (2003) Akt and PI 3-kinase signaling in cardiomyocyte hypertrophy and survival. Cell Cycle 2, 220-223.

Matsumoto, Y., Uwatoku, T., Oi, K., Abe, K., Hattori, T., Morishige, K., Eto, Y., Fukumoto, Y., Nakamura, K. I., Shibata, Y., Matsuda, T., Takeshita, A., and Shimokawa, H. (2003) Long-Term Inhibition of Rho-kinase Suppresses Neointimal Formation After Stent Implantation in Porcine Coronary Arteries—Involvement of Multiple Mechanisms. Arterioscler Thromb Vasc Biol.

Mehrhof, F. B., Muller, F. U., Bergmann, M. W., Li, P., Wang, Y., Schmitz, W., Dietz, R., and von Harsdorf, R. (2001) In cardiomyocyte hypoxia, insulin-like growth factor-I-induced antiapoptotic signaling requires phosphatidylinositol-3-OH-kinase-dependent and mitogen-activated protein kinase-dependent activation of the transcription factor cAMP response element-binding protein. Circulation 104, 2088-2094.

Moore, M., Marroquin, B. A., Gugliotta, W., Tse, R., and White, S. R. (2003) Rho Kinase Inhibition Initiates Apoptosis in Human Airway Epithelial Cells. Am J Respir Cell Mol Biol.

Moretti, A., Weig, H. J., Ott, T., Seyfarth, M., Holthoff, H. P., Grewe, D., Gillitzer, A., Bott-Flugel, L., Schomig, A., Ungerer, M., and Laugwitz, K. L. (2002) Essential myosin light chain as a target for caspase-3 in failing myocardium. Proc Natl Acad Sci USA.

Morissette, M. R., Sah, V. P., Glembotski, C. C., and Brown, J. H. (2000) The Rho effector, PKN, regulates ANF gene transcription in cardiomyocytes through a serum response element. Am J Physiol Heart Circ Physiol 278, H1769-1774.

Moses, K. A., DeMayo, F., Braun, R. M., Reecy, J. L., and Schwartz, R. J. (2001) Embryonic expression of an Nkx2-5/Cre gene using ROSA26 reporter mice. Genesis 31, 176-180.

Muyrers, J. P., Zhang, Y., Testa, G., and Stewart, A. F. (1999) Rapid modification of bacterial artificial chromosomes by ET-recombination. Nucleic Acids Res 27, 1555-1557.

Nadal-Ginard, B., Kajstura, J., Anversa, P., and Leri, A. (2003) A matter of life and death: cardiac myocyte apoptosis and regeneration. J Clin Invest 111, 1457-1459.

Nakagawa, O., Fujisawa, K., Ishizaki, T., Saito, Y., Nakao, K., and Narumiya, S. (1996) ROCK-I and ROCK-II, two isoforms of Rho-associated coiled-coil forming protein serine/threonine kinase in mice. FEBS Lett 392, 189-193.

Nakanishi, T., et al., Structure-function relationship of yeast S-Il in terms of stimulation of RNA polymerase II, arrest relief, and suppression of 6-azauracil sensitivity. J Biol Chem, 1995. 270(15): p. 8991-5.

Narula, J., Arbustini, E., Chandrashekhar, Y., and Schwaiger, M. (2001) Apoptosis and the systolic dysfunction in congestive heart failure. Story of apoptosis interruptus and zombie myocytes. Cardiol Clin 19, 113-126.

Narula, J., Haider, N., Virmani, R., DiSalvo, T. G., Kolodgie, F. D., Hajjar, R. J., Schmidt, U., Semigran, M. J., Dec, G. W., and Khaw, B. A. (1996) Apoptosis in myocytes in end-stage heart failure. N Engl J Med 335, 1182-1189.

Narula, J., Pandey, P., Arbustini, E., Haider, N., Narula, N., Kolodgie, F. D., Dal Bello, B., Semigran, M. J., Bielsa-Masdeu, A., Dec, G. W., Israels, S., Ballester, M., Virmani, R., Saxena, S., and Kharbanda, S. (1999) Apoptosis in heart failure: release of cytochrome c from mitochondria and activation of caspase-3 in human cardiomyopathy. Proc Natl Acad Sci USA 96, 8144-8149.

Nefedov, M., Williamson, R., and Ioannou, P. A. (2000) Insertion of disease-causing mutations in BACs by homologous recombination in Escherichia coli. Nucleic Acids Res 28, E79.

Negoro, S., Oh, H., Tone, E., Kunisada, K., Fujio, Y., Walsh, K., Kishimoto, T., and Yamauchi-Takihara, K. (2001) Glycoprotein 130 regulates cardiac myocyte survival in doxorubicin-induced apoptosis through phosphatidylinositol 3-kinase/Akt phosphorylation and Bcl-xL/caspase-3 interaction. Circulation 103, 555-561.

Norman, C., Runswick, M., Pollock, R., and Treisman, R. (1988) Isolation and properties of cDNA clones encoding SRF, a transcription factor that binds to the c-fos serum response element. Cell 55, 989-1003.

Ogata, Y., Takahashi, M., Takeuchi, K., Ueno, S., Mano, H., Ookawara, S., Kobayashi, E., Ikeda, U., and Shimada, K. (2002) Fluvastatin induces apoptosis in rat neonatal cardiac myocytes: a possible mechanism of statin-attenuated cardiac hypertrophy. J Cardiovasc Pharmacol 40, 907-915.

Olivetti, G., Abbi, R., Quaini, F., Kajstura, J., Cheng, W., Nitahara, J. A., Quaini, E., Di Loreto, C., Beltrami, C. A., Krajewski, S., Reed, J. C., and Anversa, P. (1997) Apoptosis in the failing human heart. N Engl J Med 336, 1131-1141.

Olson, M. F., Paterson, H. F., and Marshall, C. J. (1998) Signals from Ras and Rho GTPases interact to regulate expression of p21Waf1/Cip1. Nature 394, 295-299.

Patel, R., Nagueh, S. F., Tsybouleva, N., Abdellatif, M., Lutucuta, S., Kopelen, H. A., Quinones, M. A., Zoghbi, W. A., Entman, M. L., Roberts, R., and Marian, A. J. (2001) Simvastatin induces regression of cardiac hypertrophy and fibrosis and improves cardiac function in a transgenic rabbit model of human hypertrophic cardiomyopathy. Circulation 104, 317-324.

Peng, C. F., Wei, Y., Levsky, J. M., McDonald, T. V., Childs, G., and Kitsis, R. N. (2002) Microarray analysis of global changes in gene expression during cardiac myocyte differentiation. Physiol Genomics 9, 145-155.

Petrache, I., Crow, M. T., Neuss, M., and Garcia, J. G. (2003) Central involvement of Rho family GTPases in TNF-alpha-mediated bovine pulmonary endothelial cell apoptosis. Biochem Biophys Res Commun 306, 244-249

Pfaffl, M. W., A new mathematical model for relative quantification in real-time RT-PCR. Nucleic Acids Res, 2001. 29(9): p. e45.

Plet, A., D. Eick, and J. M. Blanchard, Elongation and premature termination of transcripts initiated from c-fos and c-myc promoters show dissimilar patterns. Oncogene, 1995. 10(2): p. 319-28.

Pu, W. T., Ma, Q., and Izumo, S. (2003) NFAT transcription factors are critical survival factors that inhibit cardiomyocyte apoptosis during phenylephrine stimulation in vitro. Circ Res 92, 725-731.

Reyes, J. C., C. Muchardt, and M. Yaniv, Components of the human SWI/SNF complex are enriched in active chromatin and are associated with the nuclear matrix. J Cell Biol, 1997. 137(2): p. 263-74.

Riento, K., and Ridley, A. J. (2003) Rocks: multifunctional kinases in cell behaviour. Nat Rev Mol Cell Biol 4, 446-456.

Sabbah, H. N. (2000) Apoptotic cell death in heart failure. Cardiovasc Res 45, 704-712.

Sabri, A., and Steinberg, S. F. (2003) Protein kinase C isoform-selective signals that lead to cardiac hypertrophy and the progression of heart failure. Mol Cell Biochem 251, 97-101.

Sabri, A., Hughie, H. H., and Lucchesi, P. A. (2003) Regulation of hypertrophic and apoptotic signaling pathways by reactive oxygen species in cardiac myocytes. Antioxid Redox Signal 5, 731-740.

Sadoshima, J., and Izumo, S. (1997) The cellular and molecular response of cardiac myocytes to mechanical stress. Annu Rev Physiol 59, 551-571.

Sah, V. P., Minamisawa, S., Tam, S. P., Wu, T. H., Dom, G. W., 2nd, Ross, J., Jr., Chien, K. R., and Brown, J. H. (1999) Cardiac-specific overexpression of RhoA results in sinus and atrioventricular nodal dysfunction and contractile failure. J Clin Invest 103, 1627-1634.

Satoh, S., Ueda, Y., Koyanagi, M., Kadokami, T., Sugano, M., Yoshikawa, Y., and Makino, N. (2003) Chronic inhibition of Rho kinase blunts the process of left ventricular hypertrophy leading to cardiac contractile dysfunction in hypertension-induced heart failure. J Mol Cell Cardiol 35, 59-70.

Sauzeau, V., Le Jeune, H., Cario-Toumaniantz, C., Vaillant, N., Gadeau, A. P., Desgranges, C., Scalbert, E., Chardin, P., Pacaud, P., and Loirand, G. (2000) P2Y(1), P2Y(2), P2Y(4), and P2Y(6) receptors are coupled to Rho and Rho kinase activation in vascular myocytes. Am J Physiol Heart Circ Physiol 278, H1751-1761.

Sauzeau, V., Le Mellionnec, E., Bertoglio, J., Scalbert, E., Pacaud, P., and Loirand, G. (2001) Human urotensin II-induced contraction and arterial smooth muscle cell proliferation are mediated by RhoA and Rho-kinase. Circ Res 88, 1102-1104.

Sawada, N., Itoh, H., Ueyama, K., Yamashita, J., Doi, K., Chun, T. H., Inoue, M., Masatsugu, K., Saito, T., Fukunaga, Y., Sakaguchi, S., Arai, H., Ohno, N., Komeda, M., and Nakao, K. (2000) Inhibition of rho-associated kinase results in suppression of neointimal formation of balloon-injured arteries. Circulation 101, 2030-2033.

Sawyer, D. B., Siwik, D. A., Xiao, L., Pimentel, D. R., Singh, K., and Colucci, W. S. (2002) Role of oxidative stress in myocardial hypertrophy and failure. J Mol Cell Cardiol 34, 379-388.

Schaffer, S., Solodushko, V., Pastukh, V., Ricci, C., and Azuma, J. (2003) Possible Cause of Taurine-deficient Cardiomyopathy: Potentiation of Angiotensin II Action. J Cardiovasc Pharmacol 41, 751-759.

Schneider, M. D., and Schwartz, R. J. (2000) Chips ahoy: gene expression in failing hearts surveyed by high-density microarrays. Circulation 102, 3026-3027.

Sebbagh, M., Renvoize, C., Hamelin, J., Riche, N., Bertoglio, J., and Breard, J. (2001) Caspase-3-mediated cleavage of ROCK I induces MLC phosphorylation and apoptotic membrane blebbing. Nat Cell Biol 3, 346-352.

Shariat, S. F., Desai, S., Song, W., Khan, T., Zhao, J., Nguyen, C., Foster, B. A., Greenberg, N., Spencer, D. M., and Slawin, K. M. (2001) Adenovirus-mediated transfer of inducible caspases: a novel “death switch” gene therapeutic approach to prostate cancer. Cancer Res 61, 2562-2571.

Steiner, M. S., et al., Antisense c-myc retroviral vector suppresses established human prostate cancer. Hum Gene Ther, 1998. 9(5): p. 747-55.

Sussman, M. A., McCulloch, A., and Borg, T. K. (2002) Dance band on the Titanic: biomechanical signaling in cardiac hypertrophy. Circ Res 91, 888-898.

Takemoto, M., Node, K., Nakagami, H., Liao, Y., Grimm, M., Takemoto, Y., Kitakaze, M., and Liao, J. K. (2001) Statins as antioxidant therapy for preventing cardiac myocyte hypertrophy. J Clin Invest 108, 1429-1437.

Takemoto, M., Sun, J., Hiroki, J., Shimokawa, H., and Liao, J. K. (2002) Rho-kinase mediates hypoxia-induced downregulation of endothelial nitric oxide synthase. Circulation 106, 57-62.

Tan, F. L., Moravec, C. S., Li, J., Apperson-Hansen, C., McCarthy, P. M., Young, J. B., and Bond, M. (2002) The gene expression fingerprint of human heart failure. Proc Natl Acad Sci USA 99, 11387-11392.

Thompson, N. E., D. B. Aronson, and R. R. Burgess, Purification of eukaryotic RNA polymerase II by immunoaffinity chromatography. Elution of active enzyme with protein stabilizing agents from a polyol-responsive monoclonal antibody. J Biol Chem, 1990. 265(12): p. 7069-77.

Thomberry, N. A., and Lazebnik, Y. (1998) Caspases: enemies within. Science 281, 1312-1316.

Thomberry, N. A., Chapman, K. T., and Nicholson, D. W. (2000) Determination of caspase specificities using a peptide combinatorial library. Methods Enzymol 322, 100-110.

Thomberry, N. A., Rano, T. A., Peterson, E. P., Rasper, D. M., Timkey, T., Garcia-Calvo, M., Houtzager, V. M., Nordstrom, P. A., Roy, S., Vaillancourt, J. P., Chapman, K. T., and Nicholson, D. W. (1997) A combinatorial approach defines specificities of members of the caspase family and granzyme B. Functional relationships established for key mediators of apoptosis. J Biol Chem 272, 17907-17911.

Thumkeo, D., Keel, J., Ishizaki, T., Hirose, M., Nonomura, K., Oshima, H., Oshima, M., Taketo, M. M., and Narumiya, S. (2003) Targeted disruption of the mouse rho-associated kinase 2 gene results in intrauterine growth retardation and fetal death. Mol Cell Biol 23, 5043-5055.

Torsoni, A. S., Fonseca, P. M., Crosara-Alberto, D. P., and Franchini, K. G. (2003) Early activation of p160ROCK by pressure overload in rat heart. Am J Physiol Cell Physiol 284, C1411-1419.

Tournier, C., Hess, P., Yang, D. D., Xu, J., Turner, T. K., Nimnual, A., Bar-Sagi, D., Jones, S. N., Flavell, R. A., and Davis, R. J. (2000) Requirement of JNK for stress-induced activation of the cytochrome c-mediated death pathway. Science 288, 870-874.

Townsend, K. J., Zhou, P., Qian, L., Bieszczad, C. K., Lowrey, C. H., Yen, A., and Craig, R. W. (1999) Regulation of MCL1 through a serum response factor/Elk-1-mediated mechanism links expression of a viability-promoting member of the BCL2 family to the induction of hematopoietic cell differentiation. J Biol Chem 274, 1801-1813.

Ueda, H., Morishita, R., Itoh, H., Narumiya, S., Mikoshiba, K., Kato, K., and Asano, T. (2001) Galphal 1 induces caspase-mediated proteolytic activation of Rho-associated kinase, ROCK-I, in HeLa cells. J Biol Chem 276, 42527-42533.

Uehata, M., Ishizaki, T., Satoh, H., Ono, T., Kawahara, T., Morishita, T., Tamakawa, H., Yamagami, K., Inui, J., Maekawa, M., and Narumiya, S. (1997) Calcium sensitization of smooth muscle mediated by a Rho-associated protein kinase in hypertension. Nature 389, 990-994.

Van Aelst, L., and D'Souza-Schorey, C. (1997) Rho GTPases and signaling networks. Genes Dev 11, 2295-2322.

Vigushin, D. M. and R. C. Coombes, Histone deacetylase inhibitors in cancer treatment. Anticancer Drugs, 2002. 13(1): p. 1-13.

Wang, Y. (2001) Signal transduction in cardiac hypertrophy—dissecting compensatory versus pathological pathways utilizing a transgenic approach. Curr Opin Pharnacol 1, 134-140.

Wang, Y., DeMayo, F. J., Tsai, S. Y., and O'Malley, B. W. (1997) Ligand-inducible and liver-specific target gene expression in transgenic mice. Nat Biotechnol 15, 239-243.

Wei, L., Imanaka-Yoshida, K., Wang, L., Zhan, S., Schneider, M. D., DeMayo, F. J., and Schwartz, R. J. (2002) Inhibition of Rho family GTPases by Rho GDP dissociation inhibitor disrupts cardiac morphogenesis and inhibits cardiomyocyte proliferation. Development 129, 1705-1714.

Wei, L., Roberts, W., Wang, L., Yamada, M., Zhang, S., Zhao, Z., Rivkees, S. A., Schwartz, R. J., and Imanaka-Yoshida, K. (2001) Rho kinases play an obligatory role in vertebrate embryonic organogenesis. Development 128, 2953-2962.

Wei, L., Wang, L., Carson, J. A., Agan, J. E., Imanaka-Yoshida, K., and Schwartz, R. J. (2001) betal integrin and organized actin filaments facilitate cardiomyocyte-specific RhoA-dependent activation of the skeletal alpha-actin promoter. Faseb J 15, 785-796.

Weir, H. K., et al., Annual report to the nation on the status of cancer, 1975-2000, featuring the uses of surveillance data for cancer prevention and control. J Natl Cancer Inst, 2003. 95(17): p. 1276-99.

Wencker, D., Chandra, M., Nguyen, K., Miao, W., Garantziotis, S., Factor, S. M., Shirani, J., Armstrong, R. C., and Kitsis, R. N. (2003) A mechanistic role for cardiac myocyte apoptosis in heart failure. J Clin Invest 111, 1497-1504.

Yamamoto, S., Yang, G., Zablocki, D., Liu, J., Hong, C., Kim, S. J., Soler, S., Odashima, M., Thaisz, J., Yehia, G., Molina, C. A., Yatani, A., Vatner, D. E., Vatner, S. F., and Sadoshima, J. (2003) Activation of Mstl causes dilated cardiomyopathy by stimulating apoptosis without compensatory ventricular myocyte hypertrophy. J Clin Invest 111, 1463-1474.

Yanazume, T., Hasegawa, K., Wada, H., Morimoto, T., Abe, M., Kawamura, T., and Sasayama, S. (2002) Rho/ROCK pathway contributes to the activation of extracellular signal-regulated kinase/GATA-4 during myocardial cell hypertrophy. J Biol Chem 277, 8618-8625.

Yoo, O. J., et al., Cloning, expression and characterization of the human transcription elongation factor, TFIIS. Nucleic Acids Res, 1991. 19(5): p. 1073-9.

Yoshida, M., T. Shimazu, and A. Matsuyama, Protein deacetylases: enzymes with functional diversity as novel therapeutic targets. Prog Cell Cycle Res, 2003. 5: p. 269-78.

Yussman, M. G., Toyokawa, T., Odley, A., Lynch, R. A., Wu, G., Colbert, M. C., Aronow, B. J., Lorenz, J. N., and Dom, G. W., 2nd (2002) Mitochondrial death protein Nix is induced in cardiac hypertrophy and triggers apoptotic cardiomyopathy. Nat Med 8, 725-730.

Zhang, X., Azhar, G., Chai, J., Sheridan, P., Nagano, K., Brown, T., Yang, J., Khrapko, K., Borras, A. M., Lawitts, J., Misra, R. P., and Wei, J. Y. (2001) Cardiomyopathy in transgenic mice with cardiac-specific overexpression of serum response factor. Am J Physiol Heart Circ Physiol 280, H1782-1792.

Zhang, X., Chai, J., Azhar, G., Sheridan, P., Borras, A. M., Furr, M. C., Khrapko, K., Lawitts, J., Misra, R. P., and Wei, J. Y. (2001) Early postnatal cardiac changes and premature death in transgenic mice overexpressing a mutant form of serum response factor. J Biol Chem 276, 40033-40040.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

We claim:

1. A method of preventing or delaying apoptosis of a cell, comprising the step of delivering an agent to the cell, wherein said agent selectively inhibits ROCK1 and wherein said agent comprises a nucleic acid, polypeptide, peptide, or mixture thereof.

2. The method of claim 1, wherein said cell is a heart cell, lung cell, liver cell, kidney cell, mesenchymal stem cell, fibroblast cell, myofibroblast cell, or stem cell.

3. The method of claim 1, wherein the agent is a caspase 3 cleavage-resistant mutant of ROCK1.

4. The method of claim 3, wherein the mutant comprises ROCK1_{D1113A}.

5. The method of claim 1, wherein the agent is a kinase-defective mutant of ROCK1.

6. The method of claim 5, wherein the kinase-defective mutant is ROCK1_{KD}.

7. The method of claim 1, wherein said nucleic acid comprises siRNA.

8. The method of claim 1, wherein said nucleic acid comprises antisense RNA.

9. The method of claim 1, wherein said agent comprises a peptide.

10. The method of claim 1, wherein said peptide comprises at least part of the pleckstrin homology domain of ROCK1.

11. The method of claim 1, wherein said agent is further defined as an agent that inhibits ROCK1 but does not inhibit ROCK2.

12. The method of claim 1, wherein said inhibiting of ROCK1 is further defined as:
inhibiting activity of ROCK1 in said cell;
inhibiting expression of ROCK1 in said cell;
inhibiting cleavage of ROCK1 in said cell;
or a combination thereof.

13. The method of claim 12, wherein said cleavage of ROCK1 is by a caspase.

14. The method of claim 13, wherein said caspase is caspase 3.

15. The method of claim 1, wherein said cell is from cardiac tissue, liver tissue, kidney tissue, lung tissue, or vasculature tissue.

16. The method of claim 1, wherein said cell is a cardiomyocyte.

17. The method of claim 1, wherein said cell is a heart cell, lung cell, liver cell, kidney cell, mesenchymal stem cell, fibroblast cell, myofibroblast cell, or stem cell.

18. The method of claim 1, wherein said cell is in a mammal.

19. The method of claim 18, wherein the mammal is a human.

20. The method of claim 11, further defined as inhibiting fibrosis in cardiac tissue of the human.

21. The method of claim 20, wherein the human has cardiac disease.

22. The method of claim 20, wherein the human is susceptible to cardiac disease.

23. The method of claim 20, further comprising the step of administering an additional cardiac disease therapy to the human.

24. The method of claim 19, wherein said inhibiting step permits maintaining the adaptive response of cardiomyocyte enlargement in the human.

25. The method of claim 19, wherein said inhibiting step is further defined as not adversely affecting the ability of the human to develop enlarged cardiomyocytes in response to pressure overload.

26. A method of treating cardiac failure in an individual, said cardiac failure the direct or indirect result of cleavage of Rho kinase in at least one cardiac cell of the individual, comprising administering to the individual a therapeutically effective amount of an agent that selectively inhibits ROCK1, wherein said agent comprises a nucleic acid, polypeptide, peptide, or mixture thereof.

27. The method of claim 26, further defined as the agent selectively inhibiting ROCK1 over ROCK2.

28. The method of claim 26, wherein said method further comprises administering an additional therapy to the individual.

29. The method of claim 28, wherein said additional therapy is drug therapy, device therapy, gene therapy, nutritional therapy, exercise therapy, or a combination thereof.

30. The method of claim 29, wherein said device therapy comprises administration of a left ventricular assist device to the individual.
